(19) United States
(12) Patent Application Publication
(10) Pub. No.: US 2026/0001864 A1
(43) Pub. Date: Jan. 1, 2026

(54) COMPOUND CONTAINING SULFAMIDE STRUCTURE, AND PREPARATION METHOD THEREFOR AND APPLICATION THEREOF, AND PHARMACEUTICAL COMPOSITION AND APPLICATION

(71) Applicant: Nanjing Shuohui Pharmatechnology Co., Ltd, Nanjing, Jiangsu Province (CN)

(72) Inventors: Wei HUANG, Nanjing, Jiangsu Province (CN); Guangfu YANG, Nanjing, Jiangsu Province (CN); Tao CHEN, Nanjing, Jiangsu Province (CN); Shuli ZHAO, Nanjing, Jiangsu Province (CN); Yi GONG, Nanjing, Jiangsu Province (CN); Mingshu WANG, Nanjing, Jiangsu Province (CN)

(73) Assignee: Nanjing Shuohui Pharmatechnology Co., Ltd, Nanjing, Jiangsu Province (CN)

(21) Appl. No.: 19/120,780

(22) PCT Filed: Oct. 26, 2021
(86) PCT No.: PCT/CN2021/126527

§ 371 Date: Apr. 13, 2025

(30) Foreign Application Priority Data
Jul. 12, 2021 (CN) 202110786023.X

(51) Int. Cl. C07D 401/14; A61K 31/4025; A61K 31/4439; A61K 31/444; A61K 31/4545; A61K 31/501; C07D 207/48; C07D 401/12; C07D 405/12; C07D 405/14; C07D 409/14

(52) U.S. Cl. CPC C07D 401/14; A61K 31/4025; A61K 31/4439; A61K 31/444; A61K 31/4545; A61K 31/501; C07D 207/48; C07D 401/12; C07D 405/12; C07D 405/14; C07D 409/14

(57) ABSTRACT

The present invention relates to the field of pharmaceuticals and discloses a compound containing a sulfamide structure, its preparation method thereof and application thereof, and pharmaceutical composition and application. The compound has the structure shown in formula (I). The compound provided by the present invention can serve as therapeutic agents, particularly as gastric acid secretion inhibitors and potassium-competitive acid blockers (P-CABs).

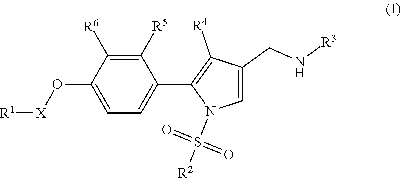

### CROSS-REFERENCE TO RELATED APPLICATION

This application claims the benefit of Chinese patent application No. 202110786023.X, filed on Jul. 12, 2021, the contents of which are incorporated herein by reference.

### TECHNICAL FIELD

The present invention relates to the field of pharmaceuticals and particularly to a compound containing a sulfamide structure, and preparation method thereof and application thereof, and pharmaceutical composition and application.

### BACKGROUND ART

The proton pump inhibitors (PPIs), represented by Omeprazole, vigorously and persistently inhibit gastric acid secretion by inhibiting the H+/K+-ATPase in gastric parietal cells. Currently, PPIs are widely used to treat acid-related diseases such as gastroesophageal reflux disease, peptic ulcer, Helicobacter pylori infection, upper gastrointestinal hemorrhage, and Zollinger-Ellison Syndrome.

Since 1988, the long-term clinical applications have revealed that PPIs have disadvantages such as instability, short half-life, susceptibility to CYP2C19 gene polymorphism, limited dosing time and slow onset of action, so there is a clinical demand for antacids with faster onset of action, stronger acid inhibition and longer pH maintenance time.

Potassium-Competitive Acid Blockers (P-CABs), as a new type of acid inhibitors, compete directly with K⁺ for the binding site on H⁺/K⁺-ATPase in the ionic form, and inhibit H⁺/K⁺-ATPase in both the resting and activated states, thereby effectively suppressing gastric acid secretion. Compared with conventional PPIs, P-CABs are characterized by lipophilicity, weak basicity, high dissociation constants and stability at a low pH value. These properties enable P-CABs to provide faster and more sustained acid suppression, reduce the incidence of nocturnal acid breakthrough, and eliminate the need for enteric coating due to their stability. Additionally, P-CABs are less affected by genetic polymorphisms.

However, the marketed representative P-CAB Vonoprazan has shown some safety risks in clinical application, such as hepatotoxicity. Therefore, although a series of potassium-competitive acid blockers have been disclosed, there is still a need to develop new compounds with richer structural types and potentially higher druggability such as better tissue distribution.

### SUMMARY OF THE INVENTION

An object of the present invention is to provide a type of new compounds with the efficacy of P-CABs in order to overcome the foregoing defects of the prior art.

To achieve the foregoing object, the first aspect of the present invention provides a compound containing a sulfamide structure, or its tautomer, mesomer, racemate, enantiomer or diastereoisomer or a mixture thereof, or a pharmaceutically acceptable salt thereof, which has a structure shown in formula (I),

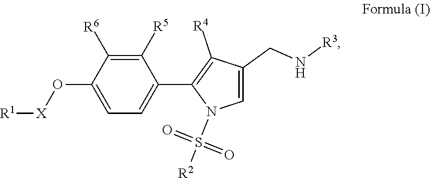

In formula (I),
X is CH₂ or CH(CH₃);
R¹ is H, substituted or unsubstituted C₁-C₄ alkyl, C₁-C₃ alkoxy, saturated 3-membered ring group containing S heteroatom, saturated 4-membered ring group containing O or S heteroatom, saturated 5-membered ring group containing O or S heteroatom, 6-membered saturated heterocycloalkyl containing 1 to 2 oxygen atoms, substituted or unsubstituted 6-membered saturated heterocycloalkyl containing 1 to 2 nitrogen atoms, substituted or unsubstituted 5-membered unsaturated heterocycloalkyl containing at least one nitrogen atom, substituted or unsubstituted 6-membered unsaturated heterocycloalkyl containing at least one nitrogen atom, —C₁-C₄ alkylidene-O—C₁-C₃ alkyl, substituted or unsubstituted C₃-C₆ saturated cycloalkyl, oxiranyl, phenyl substituted by at least two halogens, phenyl substituted by difluoromethoxy, phenyl substituted by trifluoromethoxy, phenyl substituted by carboxyl, naphthyl, phenyl substituted by at least two methyl groups, pyridin-4-yl, phenyl substituted by methoxy, thiophen-3-yl, benzodioxolyl, benzodioxanyl, —CH₂-phenyl substituted by at least two halogens, and furan-3-yl; the substituent optionally present in R¹ is selected from at least one of halogen, cyano, nitro, hydroxy, C₁-C₆ alkyl, C₁-C₆ alkoxy and C₁-C₃ aldehyde;
R² is 3-pyridyl or substituted or unsubstituted phenyl; the substituent optionally present in R² is selected from at least one of halogen, C₁-C₆ alkyl, C₁-C₆ alkoxy, —O—C₁-C₄ alkylidene-O—C₁-C₃ alkyl, C₁-C₃ alkyl substituted by 1 to 6 halogens, C₁-C₃ alkoxy substituted by 1 to 6 halogens, cyano and —O—C₁-C₄ alkylidene-C₃-C₆ cycloalkyl;
R³ is C₁-C₃ alkyl;
R⁴ is H, C₁-C₃ alkoxy or halogen;
One of R⁵ and R⁶ is H, and the other is selected from F or C₁-C₃ alkyl.

A second aspect of the present invention provides a method for preparing a compound containing a sulfamide structure, or its tautomer, mesomer, racemate, enantiomer or diastereoisomer or a mixture thereof, or a pharmaceutically acceptable salt thereof which has a structure shown in formula (I),

The method comprises: performing a contact reaction between a compound shown in formula (II) and a compound shown in formula (III);

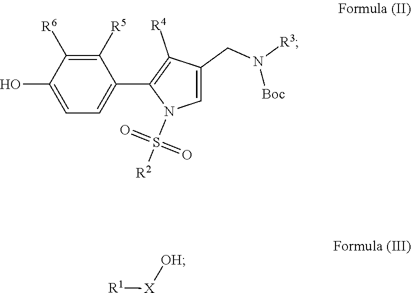

Wherein, in formula (I), formula (II) and formula (III), the definitions of each group are the same as those described in the first aspect.

A third aspect of the present invention provides a pharmaceutical composition. The pharmaceutical composition contains a therapeutically effective amount of the compound, or its tautomer, mesomer, racemate, enantiomer or diastereoisomer or a mixture thereof, or a pharmaceutically acceptable salt thereof described in the preceding first aspect. The pharmaceutical composition further contains a pharmaceutically acceptable carrier, excipient or diluent.

A fourth aspect of the present invention provides an application of the compound, or its tautomer, mesomer, racemate, enantiomer or diastereoisomer or a mixture thereof, or a pharmaceutically acceptable salt thereof described in the first aspect, or the pharmaceutical composition described in the third aspect in the preparation of an H+/K+-ATPase inhibitor medicament and/or in the preparation of a potassium-competitive acid blocker medicament.

A fifth aspect of the present invention provides an application of the compound, or its tautomer, mesomer, racemate, enantiomer or diastereoisomer or a mixture thereof, or a pharmaceutically acceptable salt thereof described in the first aspect, or the pharmaceutical composition described in the third aspect in the preparation of a medicament for treating or preventing peptic ulcer, Zollinger-Ellison Syndrome, gastritis, erosive esophagitis, reflux esophagitis, symptomatic gastroesophageal reflux disease, Barrett's esophagitis, functional dyspepsia, Helicobacter pylori infection, gastric cancer, gastric MALT lymphoma, ulcer induced by nonsteroidal anti-inflammatory drugs or post-surgical stress-induced hyperacidity or ulcer.

The foregoing compounds provided by the present invention can be used as therapeutic agents, particularly as gastric acid secretion inhibitors and P-CABs.

### DETAILED DESCRIPTION

The endpoints and any values of the ranges disclosed herein are not limited to the exact ranges or values, and such ranges or values shall be understood to include values close to such ranges or values. For numerical ranges, one or more new numerical ranges can be obtained between endpoint values of each range, between an endpoint value and a separate point value of each range, and between separate point values, and these numerical ranges shall be deemed to be specifically disclosed in the present invention.

Unless otherwise stated, the following terms used herein shall have the following meanings.

“C₁-C₆ alkyl” refers to saturated alkyl groups, including straight-chain or branched-chain alkyl groups of 1 to 6 carbon atoms, such as methyl, ethyl, n-propyl, isopropyl, n-butyl, isobutyl, tert-butyl, n-pentyl and n-hexyl. “C₁-C₄ alkyl”, etc. have similar definitions, which differ only in the total number of carbon atoms, and will not be further elaborated in the present invention.

“C₁-C₆ alkoxy” refers to saturated alkoxy groups, including straight-chain or branched-chain alkoxy groups with 1 to 6 carbon atoms, such as methoxy, ethoxy, n-propoxy, isopropoxy, n-butoxy, isobutoxy, tert-butoxy, n-pentyloxy and n-hexyloxy. “C₁-C₄ alkoxy”, “C₁-C₃ alkoxy”, etc. have similar definitions, which differ only in the total number of carbon atoms, and will not be further elaborated in the present invention.

“6-membered saturated heterocycloalkyl containing 1 to 2 oxygen atoms” means that 1 to 2 ring-forming atoms in a 6-membered saturated heterocycloalkyl are oxygen atoms, e.g., tetrahydropyranyl, dioxanyl, and the like.

“Substituted or unsubstituted C₃-C₆ cycloalkyl” means that the number of ring-forming atoms is 3 to 6, e.g., 3, 4, 5 or 6, the ring-forming atoms are all C atoms, and any of the positions in the cycloalkyl group that can be substituted can be substituted by a group as defined in the present invention.

“Substituted or unsubstituted piperidyl” means that any of the positions in the piperidine ring that can be substituted by a group as defined in the present invention.

“—O—C₁-C₄ alkylidene-O—C₁-C₂ alkyl” means that the group is linked to the parent nucleus structure by means of O and the terminal alkyl is methyl or ethyl.

“C₁-C₃ alkyl substituted by 1 to 3 halogens” means that any of the positions in C₁-C₃ alkyl that can be substituted is substituted by 1 to 3 halogens.

“—O—C₁-C₄ alkylidene-C₃-C₆ cycloalkyl” means that the group is linked to the parent nucleus structure by means of O and the terminal alkyl is C₃-C₆ cycloalkyl.

“Halogen” means fluorine, chlorine, bromine or iodine.

“Saturated 3-membered ring containing S heteroatom” refers to a group formed on the basis of a “saturated 3-membered carbon ring” in which at least one of the carbon atoms is replaced by S.

“Saturated 4-membered ring containing O or S heteroatom” means that the number of ring-forming atoms is 4, at least one of them is O or S, the remaining atoms are C atoms, and the group is a saturated ring group.

“Saturated 5-membered ring containing O or S heteroatom” means that the number of ring-forming atoms is 5, at least one of them is O or S, the remaining atoms are C atoms, and the group is a saturated ring group.

“Substituted unsaturated 5-membered ring containing at least one N atom” means that the number of ring-forming atoms is 5, at least one of them is N, the remaining atoms are C atoms, the group is an unsaturated ring group, and in any of the positions of the group that can be substituted, there is at least one substituent.

“Substituted unsaturated 6-membered ring containing at least one N atom” means that the number of ring-forming atoms is 6, at least one of them is N, the remaining atoms are C atoms, the group is an unsaturated ring group, and in any of the positions of the group that can be substituted, there is at least one substituent.

The term “optionally present” in the present invention means that the subsequently described event or situation may and may not occur, the description includes the circumstances where the event or situation may and may not occur, and that the description includes the circumstances where the event or situation occurs and does not occur.

“Pharmacologically acceptable salt” refers to the salts that retain the biological validity and properties of the parent compound. These salts include:

(1) Acid addition salts, obtained from the reaction between the free alkali of the parent compound and an inorganic acid or an organic acid, wherein the inorganic acid is hydrochloric acid, hydrobromic acid, nitric acid, phosphoric acid, metaphosphoric acid, sulfuric acid, sulfurous acid, perchloric acid, etc., and the organic acid is acetic acid, propionic acid, acrylic acid, oxalic acid, (D) or (L) malic acid, fumaric acid, maleic acid, hydroxybenzoic acid, gamma-hydroxybutyric acid, methoxybenzoic acid, phthalic acid, methanesulfonic acid, ethanesulfonic acid, naphthalene-1-sulfonic acid, naphthalene-2-sulfonic acid, p-toluene-sulfonic acid, salicylic acid, tartaric acid, citric acid, lactic acid, mandelic acid, succinic acid, malonic acid, etc.

(2) Salts formed by replacing an acidic proton present in the parent compound with a metal ion or by coordinating the proton with an organic alkali, wherein the metal ion for example is an alkali metal ion, alkaline earth metal ion or aluminum ion, and the organic alkali for example is ethanolamine, diethanolamine, triethanolamine, tromethamine or N-methylglucosamine.

“Pharmaceutical composition” refers to a mixture of one or more of the compounds in the present invention, or pharmaceutically acceptable salts, solvates, hydrates or prodrugs thereof, and other chemical components, such as pharmaceutically acceptable carriers. The purpose of the pharmaceutical composition is to facilitate the process of medicament delivery to an animal.

“Pharmaceutical carrier” refers to an inactive ingredient in a pharmaceutical composition that does not cause significant irritation to the organism and does not interfere with the biological activity and properties of the administered compound, including but not limited to: calcium carbonate, calcium phosphate, various sugars (e.g., lactose and mannitol), starch, cyclodextrin, magnesium stearate, cellulose, magnesium carbonate, acrylic polymer or methacrylic acid polymer, gel, water, polyethylene glycol, propylene glycol, ethylene glycol, castor oil or hydrogenated castor oil or polyethoxy-hydrogenated castor oil, sesame oil, corn oil and peanut oil.

As described above, the first aspect of the present invention provides a compound containing a sulfamide structure, or its tautomer, mesomer, racemate, enantiomer or diastereoisomer or a mixture thereof, or a pharmaceutically acceptable salt thereof. The compound has a structure shown in formula (I), wherein,
X is CH₂ or CH(CH₃);
R¹ is H, substituted or unsubstituted C₁-C₄ alkyl, C₁-C₃ alkoxy, saturated 3-membered ring group containing S heteroatom, saturated 4-membered ring group containing O or S heteroatom, saturated 5-membered ring group containing O or S heteroatom, 6-membered saturated heterocycloalkyl containing 1 to 2 oxygen atoms, substituted or unsubstituted 6-membered saturated heterocycloalkyl containing 1 to 2 nitrogen atoms, substituted or unsubstituted 5-membered unsaturated heterocycloalkyl containing at least one nitrogen atom, substituted or unsubstituted 6-membered unsaturated heterocycloalkyl containing at least one nitrogen atom, —C₁-C₄ alkylidene-O—C₁-C₃ alkyl, substituted or unsubstituted C₃-C₆ saturated cycloalkyl, oxiranyl, phenyl substituted by at least two halogens, phenyl substituted by difluoromethoxy, phenyl substituted by trifluoromethoxy, phenyl substituted by carboxyl, naphthyl, phenyl substituted by at least two methyl groups, pyridin-4-yl, phenyl substituted by methoxy, thiophen-3-yl, benzodioxolyl, benzodioxanyl, —CH₂-phenyl substituted by at least two halogens, furan-3-yl; the substituent optionally present in R¹ is selected from at least one of halogen, cyano, nitro, hydroxy, C₁-C₆ alkyl, C₁-C₆ alkoxy and C₁-C₃ aldehyde;
R² is 3-pyridyl or substituted or unsubstituted phenyl; the substituent optionally present in R² is selected from at least one of halogen, C₁-C₆ alkyl, C₁-C₆ alkoxy, —O—C₁-C₄ alkylidene-O—C₁-C₃ alkyl, C₁-C₃ alkyl substituted by 1 to 6 halogens, C₁-C₃ alkoxy substituted by 1 to 6 halogens, cyano and —O—C₁-C₄ alkylidene-C₃-C₆ cycloalkyl;
R³ is C₁-C₃ alkyl;
R⁴ is H, C₁-C₃ alkoxy or halogen;
One of R⁵ and R⁶ is H, and the other is selected from F and C₁-C₃ alkyl.

According to a preferred embodiment, in formula (I),
X is CH₂ or CH(CH₃);
R¹ is H, substituted or unsubstituted C₁-C₄ alkyl, C₁-C₃ alkoxy, 6-membered saturated heterocycloalkyl containing 1 to 2 oxygen atoms, substituted or unsubstituted 6-membered saturated heterocycloalkyl containing at least one nitrogen atom, —C₁-C₄ alkylidene-O—C₁-C₃ alkyl, substituted or unsubstituted C₃-C₆ saturated cycloalkyl, oxiranyl, saturated 4-membered ring group containing O atom, saturated 5-membered ring group containing O atom, substituted 5-membered unsaturated heterocycloalkyl containing at least one nitrogen atom, substituted 6-membered unsaturated heterocycloalkyl containing at least one nitrogen atom, phenyl substituted by at least two halogens, phenyl substituted by difluoromethoxy, phenyl substituted by trifluoromethoxy, phenyl substituted by carboxyl, naphthyl, phenyl substituted by at least two methyl groups, pyridin-4-yl, phenyl substituted by methoxy, thiophen-3-yl, benzodioxolyl, benzodioxanyl, —CH₂-phenyl substituted by at least two halogens, or furan-3-yl; the substituent optionally present in R¹ is selected from at least one of fluorine, chlorine, bromine, hydroxy, C₁-C₆ alkyl, C₁-C₆ alkoxy, C₁-C₂ aldehyde, cyano and nitro;
R² is 3-pyridyl or substituted or unsubstituted phenyl; the substituent optionally present in R² is selected from at least one of fluorine, chlorine, bromine, iodine, C₁-C₆ alkyl, C₁-C₆ alkoxy, —O—C₁-C₄ alkylidene-O—C₁-C₂ alkyl, C₁-C₃ alkyl substituted by 1 to 3 halogens, C₁-C₃ alkoxy substituted by 1 to 3 halogens, cyano, and —O—C₁-C₄ alkylidene-C₃-C₆ cycloalkyl;
R³ is methyl, ethyl, n-propyl or isopropyl;
R⁴ is H, methoxy, ethoxy, n-propoxy, isopropoxy, fluorine, chlorine or bromine;
One of R⁵ and R⁶ is H, and the other is selected from F, methyl, ethyl, n-propyl and isopropyl.

According to another preferred embodiment, in formula (I),
X is CH₂ or CH(CH₃);
R¹ is H, substituted or unsubstituted C₁-C₄ alkyl, C₁-C₃ alkoxy, 6-membered saturated heterocycloalkyl containing 1 to 2 oxygen atoms, substituted or unsubstituted 6-membered saturated heterocycloalkyl containing 1 to 2 nitrogen atoms, —C₁-C₄ alkylidene-O—C₁-C₃ alkyl, substituted or unsubstituted C₃-C₆ saturated cycloalkyl, oxiranyl, saturated 4-membered ring group containing O atom, saturated 5-membered ring group containing O atom, substituted unsaturated 5-membered ring containing at least one N atom, substituted unsaturated 6-membered ring containing at least one N atom, phenyl substituted by at least two halogens, phenyl substituted by difluoromethoxy, phenyl substituted by trifluoromethoxy, phenyl substituted by carboxyl, naphthyl, phenyl substituted by at least two methyl groups, pyridin-4-yl, phenyl substituted by methoxy, thiophen-3-yl, benzodioxolyl, benzodioxanyl, —CH₂-phenyl substituted by at least two halogens, or furan-3-yl; the substituent optionally present in R¹ is selected from at least one of fluorine, chlorine, bromine, hydroxy, C₁-C₄ alkyl, C₁-C₄ alkoxy and acetaldehyde;
R² is 3-pyridyl or substituted or unsubstituted phenyl; the substituent optionally present in R² is selected from at least one of fluorine, chlorine, bromine, C₁-C₆ alkyl, C₁-C₆ alkoxy, —O—C₁-C₄ alkylidene-O—C₁-C₂ alkyl, C₁-C₃ alkyl substituted by 1 to 3 halogens, C₁-C₃ alkoxy substituted by 1 to 3 halogens, cyano and —O—C₁-C₄ alkylidene-C₃-C₆ cycloalkyl;
R³ is methyl or ethyl;
R⁴ is H, methoxy or fluorine;
One of R⁵ and R⁶ is H, and the other is selected from F, methyl, ethyl, n-propyl and isopropyl.

According to still another preferred specific embodiment, in formula (I),
X is CH₂ or CH(CH₃);
R¹ is H, methyl, isopropyl, tert-butyl, hydroxy-substituted tert-butyl, difluoromethyl, methoxy, dioxanyl, substituted or unsubstituted piperidyl, —C₁-C₄ alkylidene-O—C₁-C₃ alkyl, substituted or unsubstituted C₃-C₆ saturated cycloalkyl, oxiranyl, saturated 4-membered ring group containing O atom, saturated 5-membered ring group containing O atom, substituted unsaturated 5-membered ring group containing at least one N atom, substituted unsaturated 6-membered ring group containing at least one N atom, phenyl substituted by at least two halogens, phenyl substituted by difluoromethoxy, phenyl substituted by trifluoromethoxy, phenyl substituted by carboxyl, naphthyl, phenyl substituted by at least two methyl groups, pyridin-4-yl, phenyl substituted by methoxy, thiophen-3-yl, benzodioxolyl, benzodioxanyl, —CH₂-phenyl substituted by at least two halogens, or furan-3-yl; the substituent optionally present in R¹ is selected from at least one of fluorine, chlorine, bromine, hydroxy, methyl, ethyl, n-propyl, isopropyl, n-butyl, isobutyl, tert-butyl, methoxy, ethoxy, n-propoxy, isopropoxy, n-butoxy, isobutoxy, tert-butoxy and acetaldehyde;
R² is 3-pyridyl or substituted or unsubstituted phenyl; the substituent optionally present in R² is selected from at least one of fluorine, chlorine, bromine, C₁-C₆ alkyl, C₁-C₆ alkoxy, —O—C₁-C₄ alkylidene-O—C₁-C₂ alkyl, C₁-C₃ alkyl substituted by 1 to 3 halogens, C₁-C₃ alkoxy substituted by 1 to 3 halogens, cyano, and —O—C₁-C₄ alkylidene-C₃-C₆ cycloalkyl;
R³ is methyl or ethyl;
R⁴ is H, methoxy or fluorine;
One of R⁵ and R⁶ is H, and the other is selected from F, methyl, ethyl, n-propyl and isopropyl.

According to a particularly preferred specific embodiment, the compound shown in formula (I) is selected from any of the following:

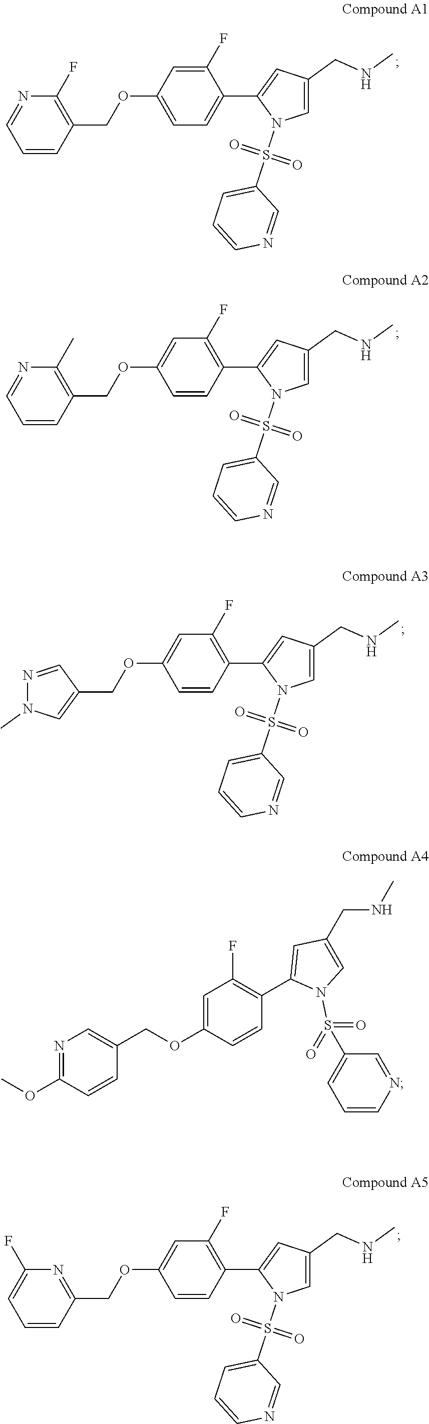

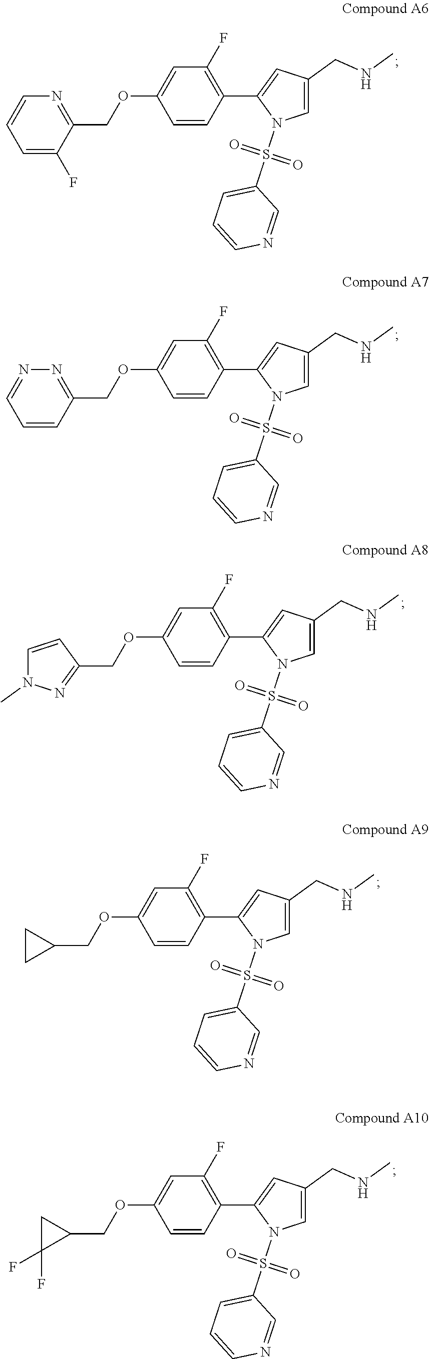

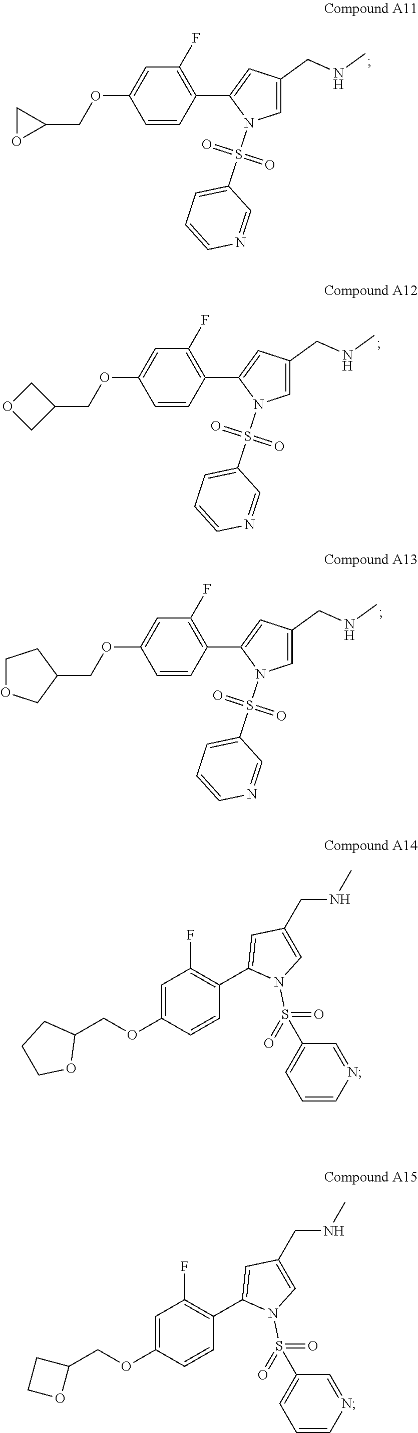 [SMILES: CNCc1cc(-c2ccc(OCC3CO3)cc2F)n(S(=O)(=O)c2cccnc2)c1 ; CNCc1cc(-c2ccc(OCC3COC3)cc2F)n(S(=O)(=O)c2cccnc2)c1 ; CNCc1cc(-c2ccc(OCC3CCOC3)cc2F)n(S(=O)(=O)c2cccnc2)c1 ; CNCc1cc(-c2ccc(OCC3CCCO3)cc2F)n(S(=O)(=O)c2cccnc2)c1 ; CNCc1cc(-c2ccc(OCC3CCO3)cc2F)n(S(=O)(=O)c2cccnc2)c1]

| Compound | |
|---|---|
| No. | Structure |
| 1 | 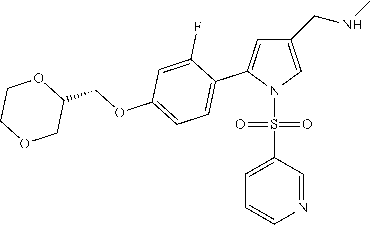 |
| 2 | 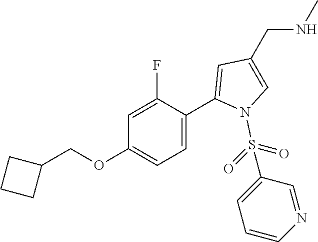 [SMILES: CNCc1cc(-c2ccc(OCC3CCC3)cc2F)n(S(=O)(=O)c2cccnc2)c1] |
| 3 | 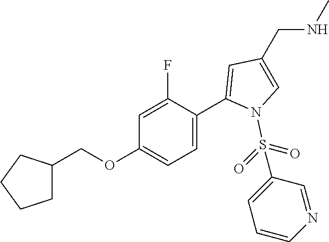 [SMILES: CNCc1cc(-c2ccc(OCC3CCCC3)cc2F)n(S(=O)(=O)c2cccnc2)c1] |
| 4 | 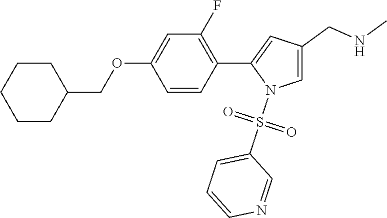 [SMILES: CNCc1cc(-c2ccc(OCC3CCCCC3)cc2F)n(S(=O)(=O)c2cccnc2)c1] |
| 5 | 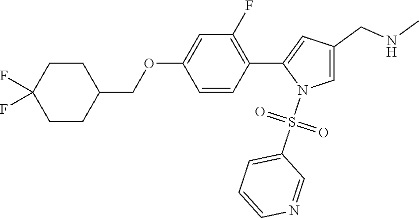 [SMILES: CNCc1cc(-c2ccc(OCC3CCC(F)(F)CC3)cc2F)n(S(=O)(=O)c2cccnc2)c1] |
| 6 | 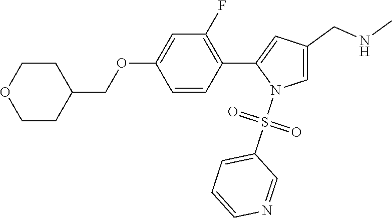 [SMILES: CNCc1cc(-c2ccc(OCC3CCOCC3)cc2F)n(S(=O)(=O)c2cccnc2)c1] |
| 7 | 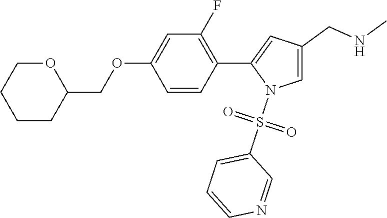 [SMILES: CNCc1cc(-c2ccc(OCC3CCCCO3)cc2F)n(S(=O)(=O)c2cccnc2)c1] |
| 8 | 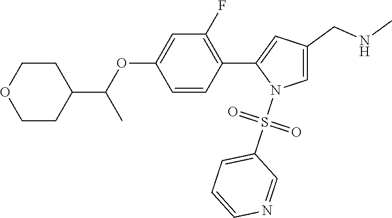 [SMILES: CNCc1cc(-c2ccc(OC(C)C3CCOCC3)cc2F)n(S(=O)(=O)c2cccnc2)c1] |
| 9 | 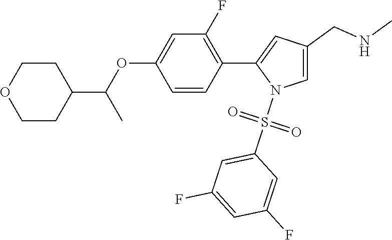 [SMILES: CNCc1cc(-c2ccc(OC(C)C3CCOCC3)cc2F)n(S(=O)(=O)c2cc(F)cc(F)c2)c1] |
| 10 | 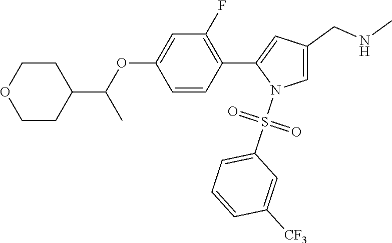 [SMILES: CNCc1cc(-c2ccc(OC(C)C3CCOCC3)cc2F)n(S(=O)(=O)c2cccc(C(F)(F)F)c2)c1] |
| 11 | 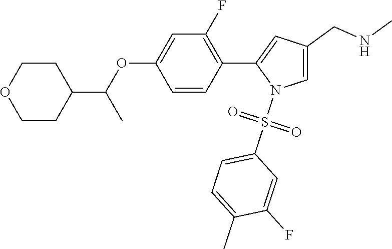 [SMILES: CNCc1cc(-c2ccc(OC(C)C3CCOCC3)cc2F)n(S(=O)(=O)c2ccc(C)c(F)c2)c1] |
| 12 | 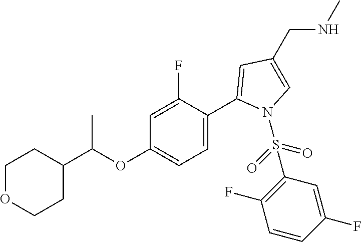 [SMILES: CNCc1cc(-c2ccc(OC(C)C3CCOCC3)cc2F)n(S(=O)(=O)c2cc(F)ccc2F)c1] |
| 13 | 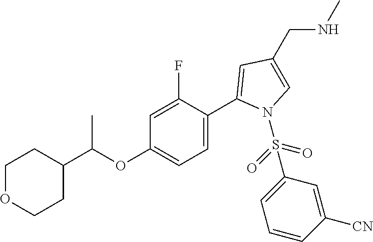 [SMILES: CNCc1cc(-c2ccc(OC(C)C3CCOCC3)cc2F)n(S(=O)(=O)c2cccc(C#N)c2)c1] |
| 14 | 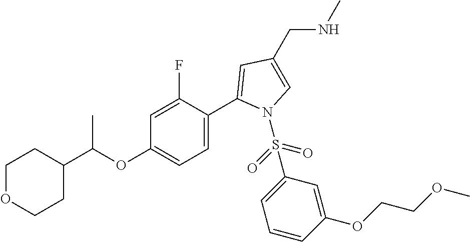 [SMILES: CNCc1cc(-c2ccc(OC(C)C3CCOCC3)cc2F)n(S(=O)(=O)c2cccc(OCCOC)c2)c1] |
| 15 | 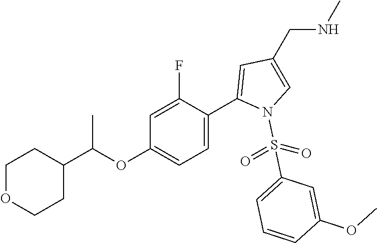 [SMILES: CNCc1cc(-c2ccc(OC(C)C3CCOCC3)cc2F)n(S(=O)(=O)c2cccc(OC)c2)c1] |
| 16 | 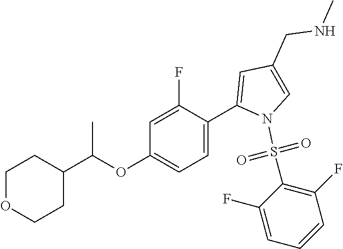 [SMILES: CNCc1cc(-c2ccc(OC(C)C3CCOCC3)cc2F)n(S(=O)(=O)c2c(F)cccc2F)c1] |
| 17 | 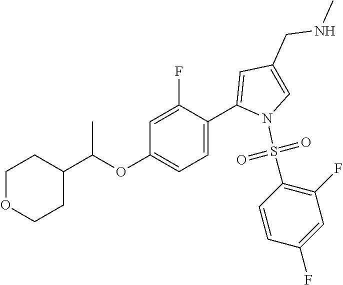 [SMILES: CNCc1cc(-c2ccc(OC(C)C3CCOCC3)cc2F)n(S(=O)(=O)c2ccc(F)cc2F)c1] |
| 18 | 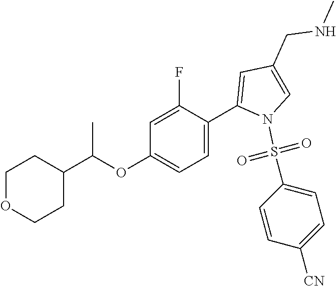 [SMILES: CNCc1cc(-c2ccc(OC(C)C3CCOCC3)cc2F)n(S(=O)(=O)c2ccc(C#N)cc2)c1] |
| 19 | 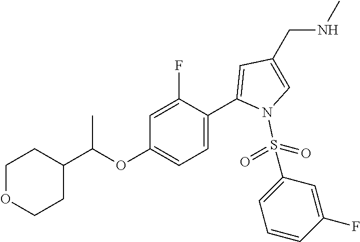 [SMILES: CNCc1cc(-c2ccc(OC(C)C3CCOCC3)cc2F)n(S(=O)(=O)c2cccc(F)c2)c1] |
| 20 | 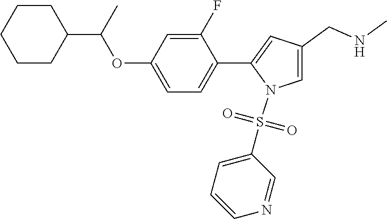 [SMILES: CNCc1cc(-c2ccc(OC(C)C3CCCCC3)cc2F)n(S(=O)(=O)c2cccnc2)c1] |
| 21 | 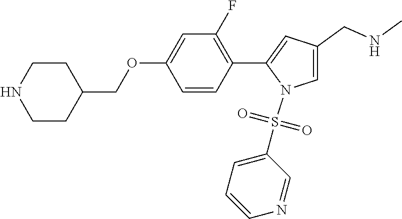 [SMILES: CNCc1cc(-c2ccc(OCC3CCNCC3)cc2F)n(S(=O)(=O)c2cccnc2)c1] |
| 22 | 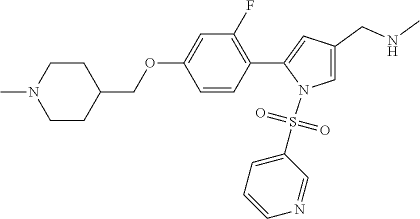 |
| 23 | 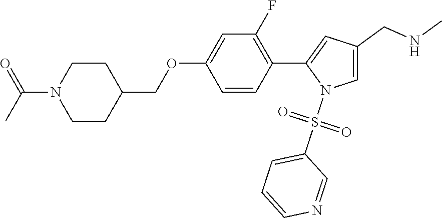 [SMILES: CNCc1cc(-c2ccc(OCC3CCN(C(C)=O)CC3)cc2F)n(S(=O)(=O)c2cccnc2)c1] |
| 24 | 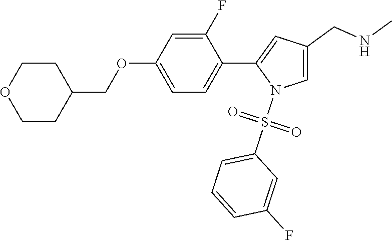 [SMILES: CNCc1cc(-c2ccc(OCC3CCOCC3)cc2F)n(S(=O)(=O)c2cccc(F)c2)c1] |
| 25 | 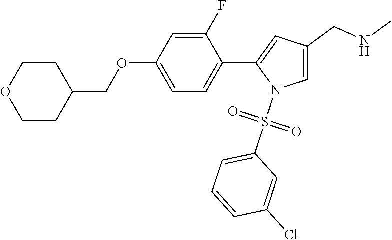 [SMILES: CNCc1cc(-c2ccc(OCC3CCOCC3)cc2F)n(S(=O)(=O)c2cccc(Cl)c2)c1] |
| 26 | 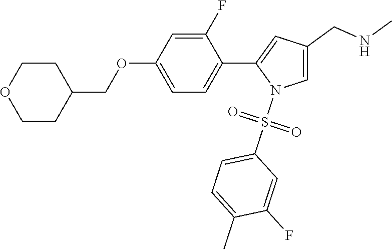 [SMILES: CNCc1cc(-c2ccc(OCC3CCOCC3)cc2F)n(S(=O)(=O)c2ccc(C)c(F)c2)c1] |
| 27 | 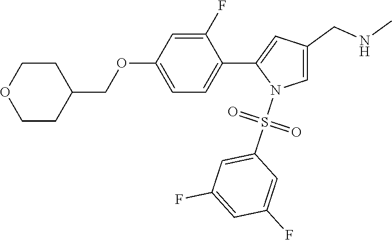 [SMILES: CNCc1cc(-c2ccc(OCC3CCOCC3)cc2F)n(S(=O)(=O)c2cc(F)cc(F)c2)c1] |
| 28 | 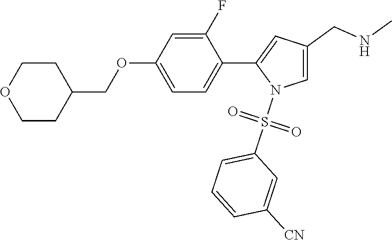 [SMILES: CNCc1cc(-c2ccc(OCC3CCOCC3)cc2F)n(S(=O)(=O)c2cccc(C#N)c2)c1] |
| 29 | 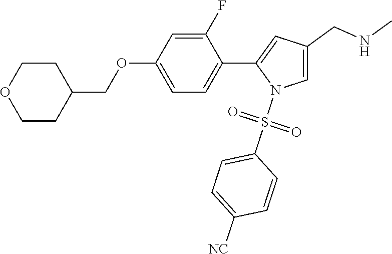 [SMILES: CNCc1cc(-c2ccc(OCC3CCOCC3)cc2F)n(S(=O)(=O)c2ccc(C#N)cc2)c1] |
| 30 | 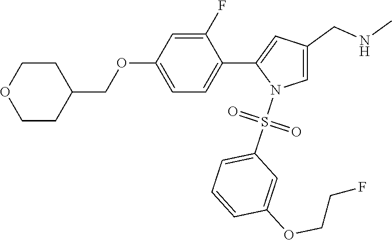 [SMILES: CNCc1cc(-c2ccc(OCC3CCOCC3)cc2F)n(S(=O)(=O)c2cccc(OCCF)c2)c1] |
| 31 | 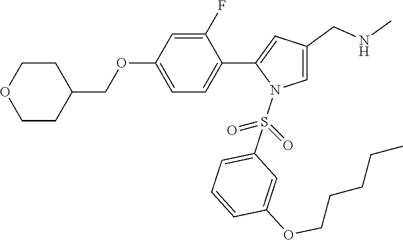 [SMILES: CCCCCOc1cccc(S(=O)(=O)n2cc(CNC)cc2-c2ccc(OCC3CCOCC3)cc2F)c1] |
| 32 | 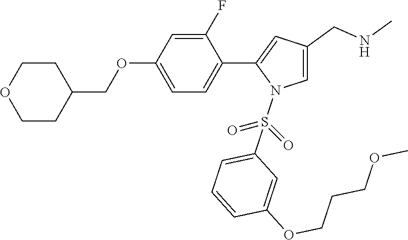 [SMILES: CNCc1cc(-c2ccc(OCC3CCOCC3)cc2F)n(S(=O)(=O)c2cccc(OCCCOC)c2)c1] |
| 33 | 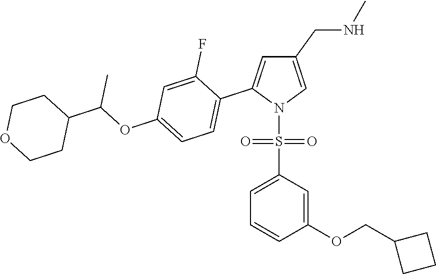 [SMILES: CNCc1cc(-c2ccc(OC(C)C3CCOCC3)cc2F)n(S(=O)(=O)c2cccc(OCC3CCC3)c2)c1] |
| 34 | 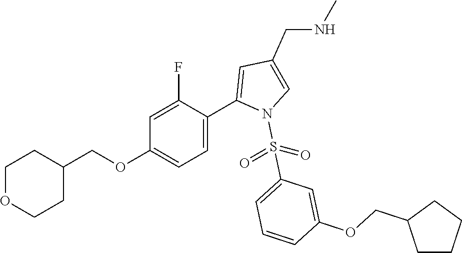 [SMILES: CNCc1cc(-c2ccc(OCC3CCOCC3)cc2F)n(S(=O)(=O)c2cccc(OCC3CCCC3)c2)c1] |
| 35 | 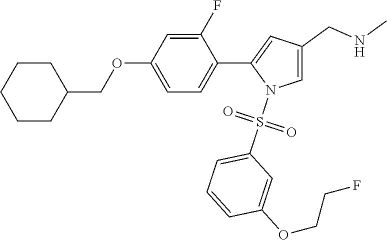 [SMILES: CNCc1cc(-c2ccc(OCC3CCCCC3)cc2F)n(S(=O)(=O)c2cccc(OCCF)c2)c1] |
| 36 | 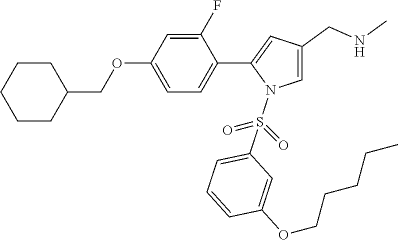 [SMILES: CCCCCOc1cccc(S(=O)(=O)n2cc(CNC)cc2-c2ccc(OCC3CCCCC3)cc2F)c1] |
| 37 | 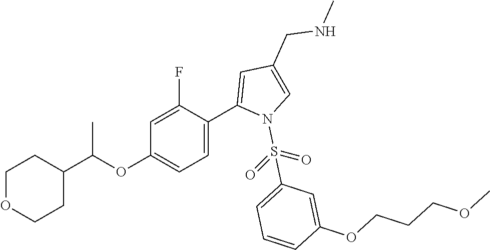 [SMILES: CNCc1cc(-c2ccc(OC(C)C3CCOCC3)cc2F)n(S(=O)(=O)c2cccc(OCCCOC)c2)c1] |
| 38 | 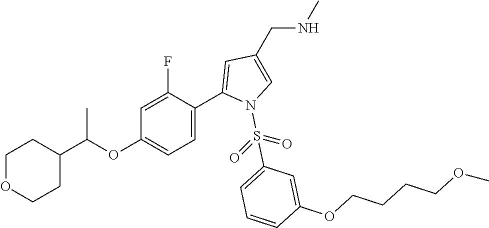 [SMILES: CNCc1cc(-c2ccc(OC(C)C3CCOCC3)cc2F)n(S(=O)(=O)c2cccc(OCCCCOC)c2)c1] |
| 39 | 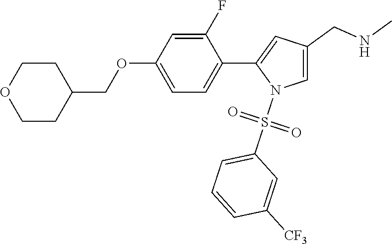 [SMILES: CNCc1cc(-c2ccc(OCC3CCOCC3)cc2F)n(S(=O)(=O)c2cccc(C(F)(F)F)c2)c1] |
| 40 | 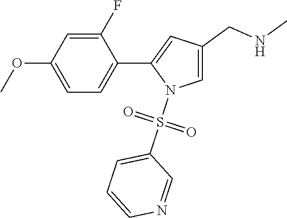 |
| 41 | 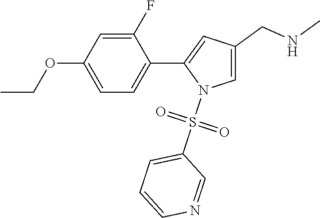 [SMILES: CCOc1ccc(-c2cc(CNC)cn2S(=O)(=O)c2cccnc2)c(F)c1] |
| 42 | 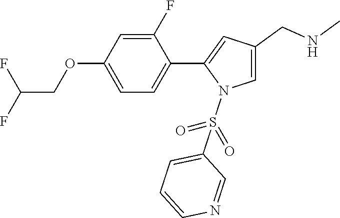 [SMILES: CNCc1cc(-c2ccc(OCC(F)F)cc2F)n(S(=O)(=O)c2cccnc2)c1] |
| 43 | 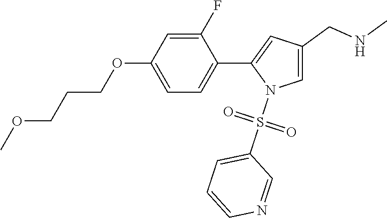 |
| 44 | 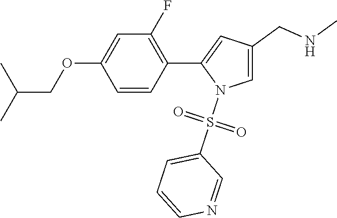 [SMILES: CNCc1cc(-c2ccc(OCC(C)C)cc2F)n(S(=O)(=O)c2cccnc2)c1] |
| 45 | 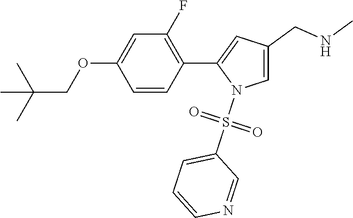 [SMILES: CNCc1cc(-c2ccc(OCC(C)(C)C)cc2F)n(S(=O)(=O)c2cccnc2)c1] |
| 46 | 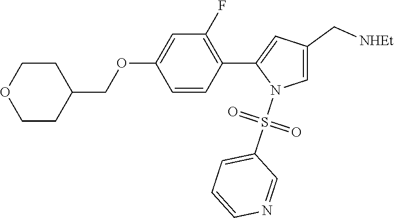 |
| 47 | 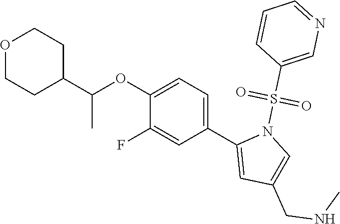 [SMILES: CNCc1cc(-c2ccc(OC(C)C3CCOCC3)c(F)c2)n(S(=O)(=O)c2cccnc2)c1] |
| 48 | 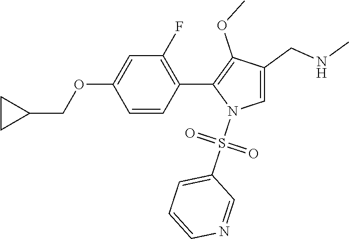 [SMILES: CNCc1cn(S(=O)(=O)c2cccnc2)c(-c2ccc(OCC3CC3)cc2F)c1OC] |
| 49 | 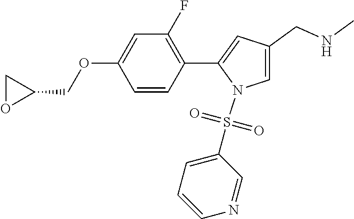 [SMILES: CNCc1cc(-c2ccc(OC[C@H]3CO3)cc2F)n(S(=O)(=O)c2cccnc2)c1] |
| 50 | 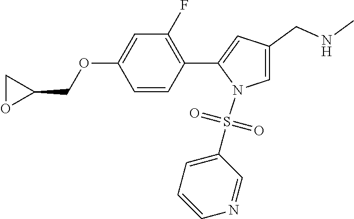 [SMILES: CNCc1cc(-c2ccc(OC[C@@H]3CO3)cc2F)n(S(=O)(=O)c2cccnc2)c1] |
| 51 | 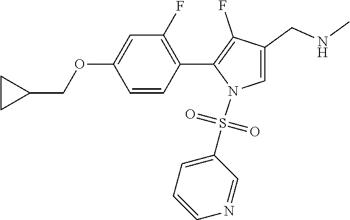 [SMILES: CNCc1cn(S(=O)(=O)c2cccnc2)c(-c2ccc(OCC3CC3)cc2F)c1F] |
| 52 | 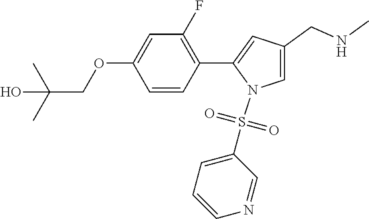 [SMILES: CNCc1cc(-c2ccc(OCC(C)(C)O)cc2F)n(S(=O)(=O)c2cccnc2)c1] |
| 53 | 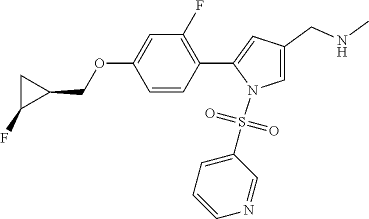 [SMILES: CNCc1cc(-c2ccc(OC[C@@H]3C[C@@H]3F)cc2F)n(S(=O)(=O)c2cccnc2)c1] |
| 54 | 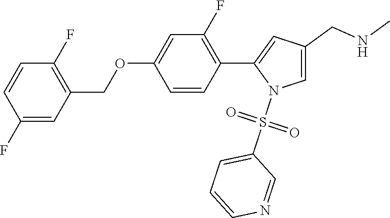 [SMILES: CNCc1cc(-c2ccc(OCc3cc(F)ccc3F)cc2F)n(S(=O)(=O)c2cccnc2)c1] |
| 55 | 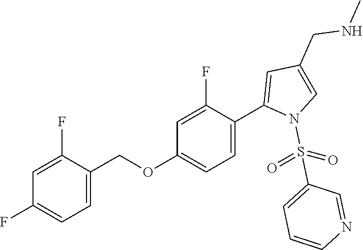 [SMILES: CNCc1cc(-c2ccc(OCc3ccc(F)cc3F)cc2F)n(S(=O)(=O)c2cccnc2)c1] |
| 56 | 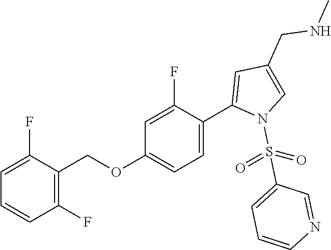 [SMILES: CNCc1cc(-c2ccc(OCc3c(F)cccc3F)cc2F)n(S(=O)(=O)c2cccnc2)c1] |
| 57 | 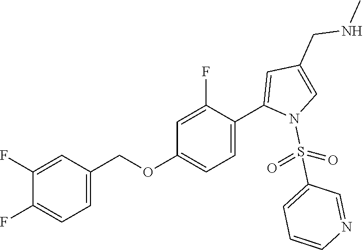 [SMILES: CNCc1cc(-c2ccc(OCc3ccc(F)c(F)c3)cc2F)n(S(=O)(=O)c2cccnc2)c1] |
| 58 | 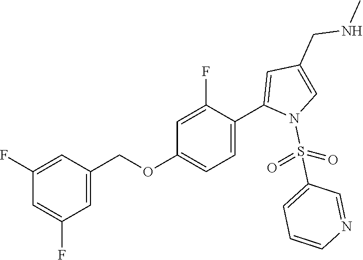 [SMILES: CNCc1cc(-c2ccc(OCc3cc(F)cc(F)c3)cc2F)n(S(=O)(=O)c2cccnc2)c1] |
| 59 | 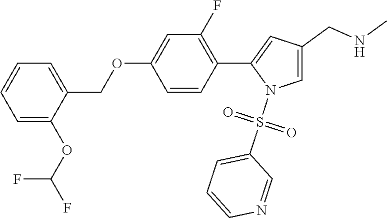 [SMILES: CNCc1cc(-c2ccc(OCc3ccccc3OC(F)F)cc2F)n(S(=O)(=O)c2cccnc2)c1] |
| 60 | 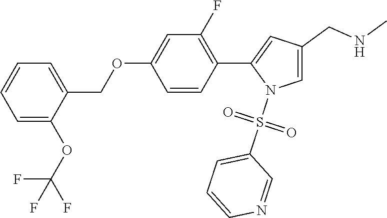 [SMILES: CNCc1cc(-c2ccc(OCc3ccccc3OC(F)(F)F)cc2F)n(S(=O)(=O)c2cccnc2)c1] |
| 61 | 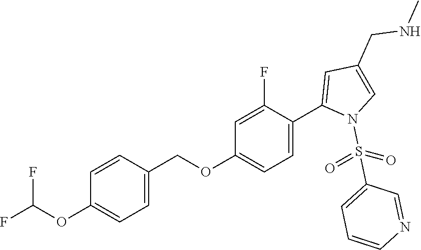 [SMILES: CNCc1cc(-c2ccc(OCc3ccc(OC(F)F)cc3)cc2F)n(S(=O)(=O)c2cccnc2)c1] |
| 62 | 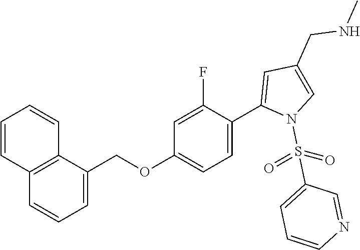 [SMILES: CNCc1cc(-c2ccc(OCc3cccc4ccccc34)cc2F)n(S(=O)(=O)c2cccnc2)c1] |
| 63 | 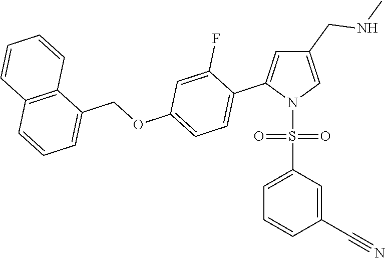 [SMILES: CNCc1cc(-c2ccc(OCc3cccc4ccccc34)cc2F)n(S(=O)(=O)c2cccc(C#N)c2)c1] |
| 64 | 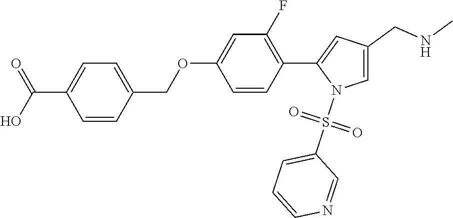 [SMILES: CNCc1cc(-c2ccc(OCc3ccc(C(=O)O)cc3)cc2F)n(S(=O)(=O)c2cccnc2)c1] |
| 65 | 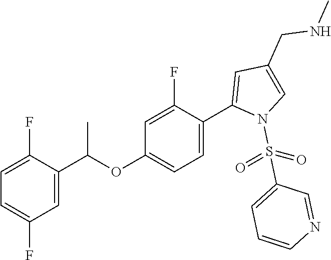 [SMILES: CNCc1cc(-c2ccc(OC(C)c3cc(F)ccc3F)cc2F)n(S(=O)(=O)c2cccnc2)c1] |
| 66 | 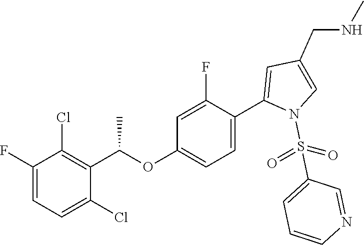 [SMILES: CNCc1cc(-c2ccc(O[C@@H](C)c3c(Cl)ccc(F)c3Cl)cc2F)n(S(=O)(=O)c2cccnc2)c1] |
| 67 | 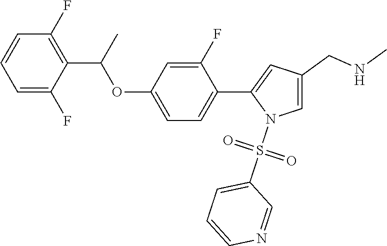 [SMILES: CNCc1cc(-c2ccc(OC(C)c3c(F)cccc3F)cc2F)n(S(=O)(=O)c2cccnc2)c1] |
| 68 | 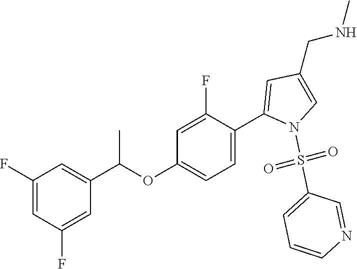 [SMILES: CNCc1cc(-c2ccc(OC(C)c3cc(F)cc(F)c3)cc2F)n(S(=O)(=O)c2cccnc2)c1] |
| 69 | 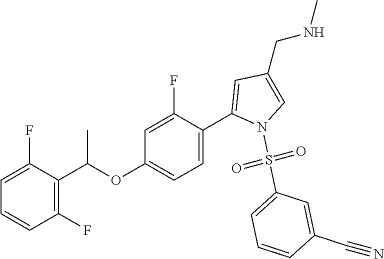 [SMILES: CNCc1cc(-c2ccc(OC(C)c3c(F)cccc3F)cc2F)n(S(=O)(=O)c2cccc(C#N)c2)c1] |
| 70 | 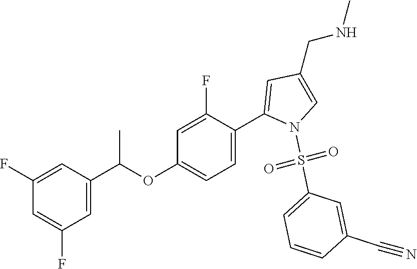 [SMILES: CNCc1cc(-c2ccc(OC(C)c3cc(F)cc(F)c3)cc2F)n(S(=O)(=O)c2cccc(C#N)c2)c1] |
| 71 | 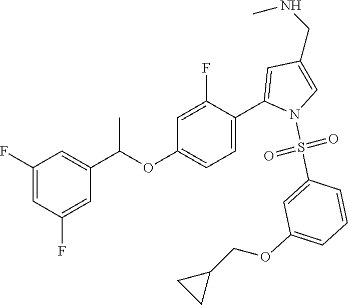 [SMILES: CNCc1cc(-c2ccc(OC(C)c3cc(F)cc(F)c3)cc2F)n(S(=O)(=O)c2cccc(OCC3CC3)c2)c1] |
| 72 | 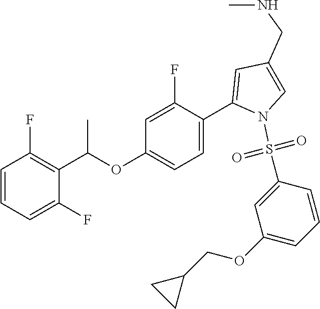 [SMILES: CNCc1cc(-c2ccc(OC(C)c3c(F)cccc3F)cc2F)n(S(=O)(=O)c2cccc(OCC3CC3)c2)c1] |
| 73 | 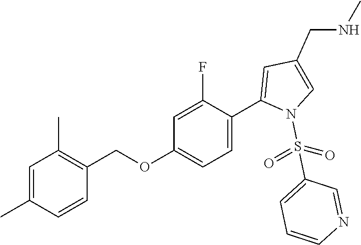 |
| 74 | 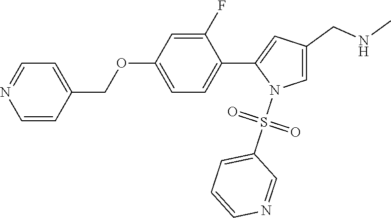 [SMILES: CNCc1cc(-c2ccc(OCc3ccncc3)cc2F)n(S(=O)(=O)c2cccnc2)c1] |
| 75 | 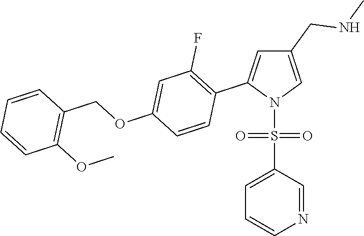 [SMILES: CNCc1cc(-c2ccc(OCc3ccccc3OC)cc2F)n(S(=O)(=O)c2cccnc2)c1] |
| 76 | 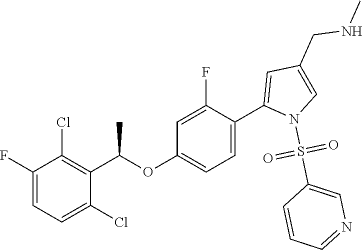 [SMILES: CNCc1cc(-c2ccc(O[C@H](C)c3c(Cl)ccc(F)c3Cl)cc2F)n(S(=O)(=O)c2cccnc2)c1] |
| 77 | 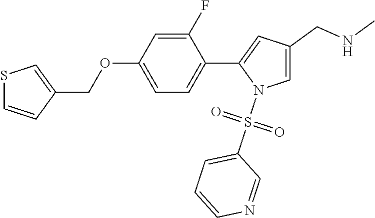 [SMILES: CNCc1cc(-c2ccc(OCc3ccsc3)cc2F)n(S(=O)(=O)c2cccnc2)c1] |
| 78 | 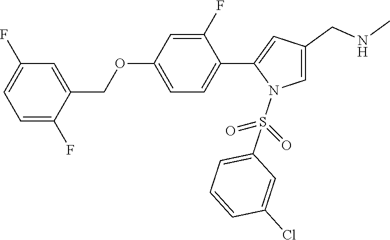 [SMILES: CNCc1cc(-c2ccc(OCc3cc(F)ccc3F)cc2F)n(S(=O)(=O)c2cccc(Cl)c2)c1] |
| 79 | 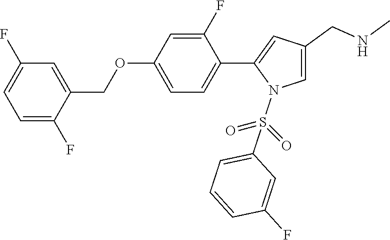 [SMILES: CNCc1cc(-c2ccc(OCc3cc(F)ccc3F)cc2F)n(S(=O)(=O)c2cccc(F)c2)c1] |
| 80 | 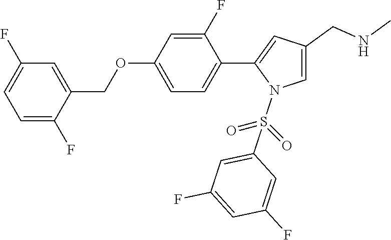 [SMILES: CNCc1cc(-c2ccc(OCc3cc(F)ccc3F)cc2F)n(S(=O)(=O)c2cc(F)cc(F)c2)c1] |
| 81 | 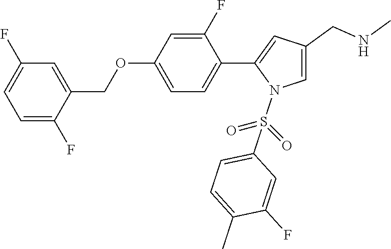 [SMILES: CNCc1cc(-c2ccc(OCc3cc(F)ccc3F)cc2F)n(S(=O)(=O)c2ccc(C)c(F)c2)c1] |
| 82 | 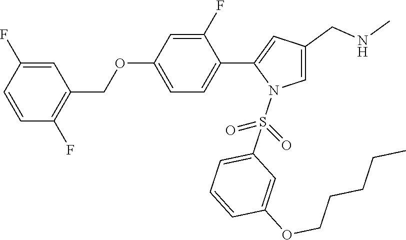 [SMILES: CCCCCOc1cccc(S(=O)(=O)n2cc(CNC)cc2-c2ccc(OCc3cc(F)ccc3F)cc2F)c1] |
| 83 | 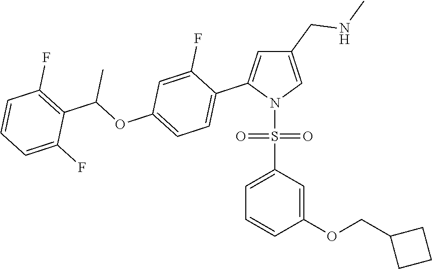 [SMILES: CNCc1cc(-c2ccc(OC(C)c3c(F)cccc3F)cc2F)n(S(=O)(=O)c2cccc(OCC3CCC3)c2)c1] |
| 84 | 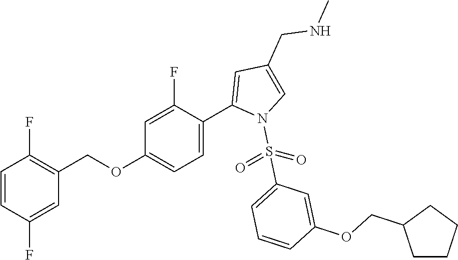 [SMILES: CNCc1cc(-c2ccc(OCc3cc(F)ccc3F)cc2F)n(S(=O)(=O)c2cccc(OCC3CCCC3)c2)c1] |
| 85 | 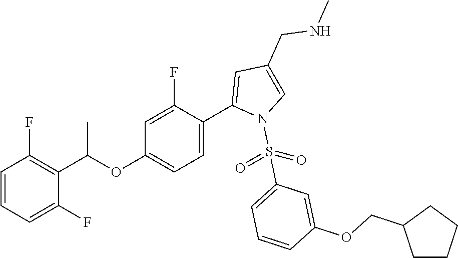 [SMILES: CNCc1cc(-c2ccc(OC(C)c3c(F)cccc3F)cc2F)n(S(=O)(=O)c2cccc(OCC3CCCC3)c2)c1] |
| 86 | 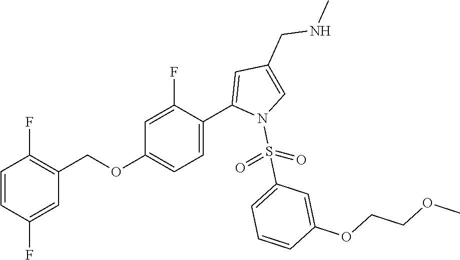 [SMILES: CNCc1cc(-c2ccc(OCc3cc(F)ccc3F)cc2F)n(S(=O)(=O)c2cccc(OCCOC)c2)c1] |
| 87 | 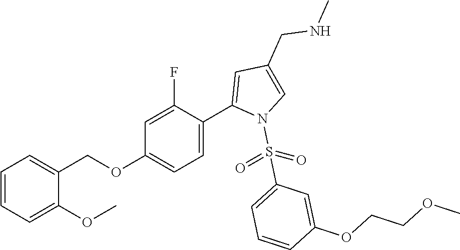 [SMILES: CNCc1cc(-c2ccc(OCc3ccccc3OC)cc2F)n(S(=O)(=O)c2cccc(OCCOC)c2)c1] |
| 88 | 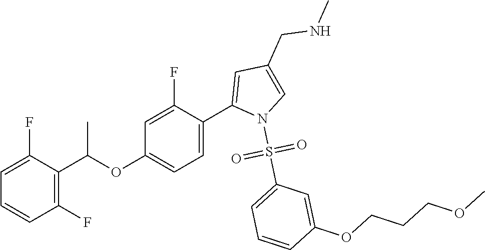 [SMILES: CNCc1cc(-c2ccc(OC(C)c3c(F)cccc3F)cc2F)n(S(=O)(=O)c2cccc(OCCCOC)c2)c1] |
| 89 | 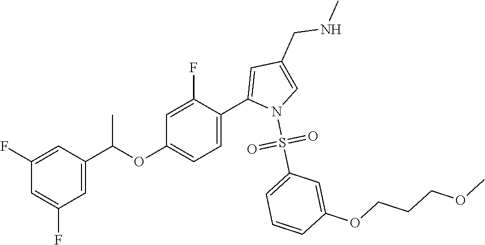 [SMILES: CNCc1cc(-c2ccc(OC(C)c3cc(F)cc(F)c3)cc2F)n(S(=O)(=O)c2cccc(OCCCOC)c2)c1] |
| 90 | 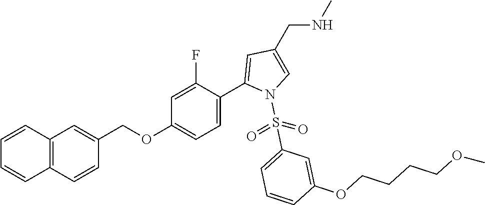 [SMILES: CNCc1cc(-c2ccc(OCc3ccc4ccccc4c3)cc2F)n(S(=O)(=O)c2cccc(OCCCCOC)c2)c1] |
| 91 | 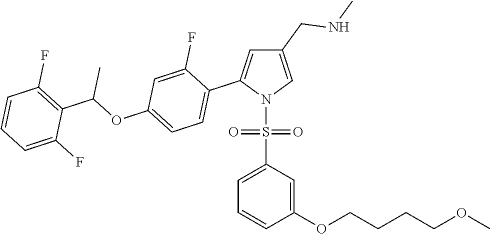 [SMILES: CNCc1cc(-c2ccc(OC(C)c3c(F)cccc3F)cc2F)n(S(=O)(=O)c2cccc(OCCCCOC)c2)c1] |
| 92 | 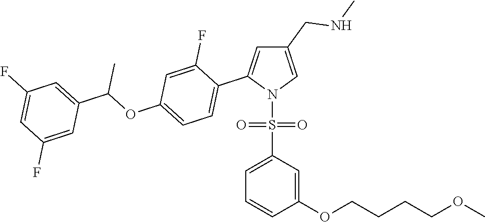 [SMILES: CNCc1cc(-c2ccc(OC(C)c3cc(F)cc(F)c3)cc2F)n(S(=O)(=O)c2cccc(OCCCCOC)c2)c1] |
| 93 | 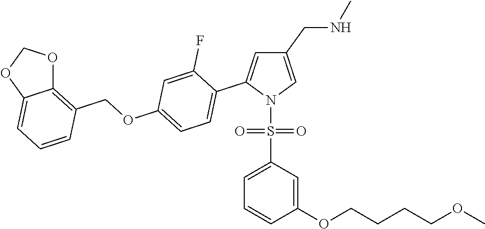 [SMILES: CNCc1cc(-c2ccc(OCc3cccc4c3OCO4)cc2F)n(S(=O)(=O)c2cccc(OCCCCOC)c2)c1] |
| 94 | 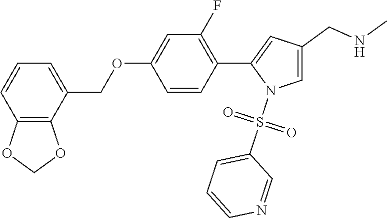 [SMILES: CNCc1cc(-c2ccc(OCc3cccc4c3OCO4)cc2F)n(S(=O)(=O)c2cccnc2)c1] |
| 95 | 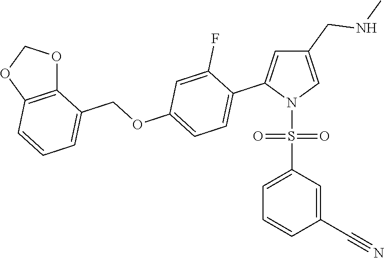 [SMILES: CNCc1cc(-c2ccc(OCc3cccc4c3OCO4)cc2F)n(S(=O)(=O)c2cccc(C#N)c2)c1] |
| 96 | 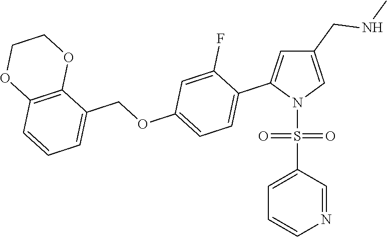 [SMILES: CNCc1cc(-c2ccc(OCc3cccc4c3OCCO4)cc2F)n(S(=O)(=O)c2cccnc2)c1] |
| 97 | 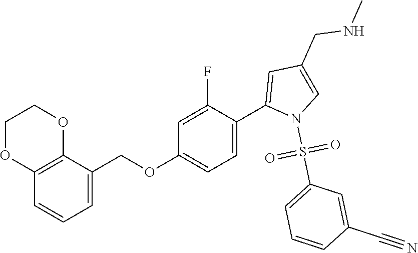 [SMILES: CNCc1cc(-c2ccc(OCc3cccc4c3OCCO4)cc2F)n(S(=O)(=O)c2cccc(C#N)c2)c1] |
| 98 | 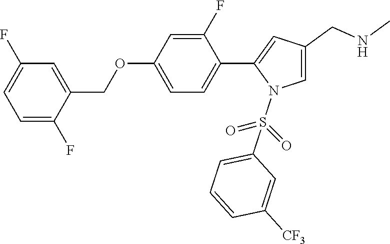 [SMILES: CNCc1cc(-c2ccc(OCc3cc(F)ccc3F)cc2F)n(S(=O)(=O)c2cccc(C(F)(F)F)c2)c1] |
| 99 | 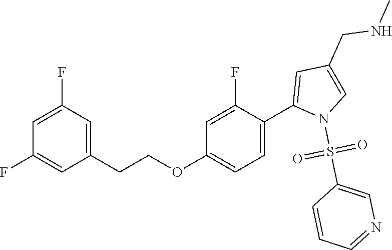 [SMILES: CNCc1cc(-c2ccc(OCCc3cc(F)cc(F)c3)cc2F)n(S(=O)(=O)c2cccnc2)c1] |
| 100 | 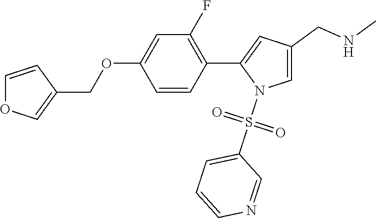 [SMILES: CNCc1cc(-c2ccc(OCc3ccoc3)cc2F)n(S(=O)(=O)c2cccnc2)c1] |

The pharmacologically acceptable salt with a structure shown in formula (I) in the present invention includes, but is not limited to, the hydrochloride form.

The present invention has no particular requirement for a specific method of preparing the compounds described in the preceding first aspect, and those skilled in the art can readily determine a suitable preparation method based on the characteristics of the structural formulae provided by the present invention in conjunction with the known technical means in the field of organic synthesis. Further, the section of examples in the present invention exemplarily enumerates the preparation methods for some of the specific compounds, and those skilled in the art can determine specific preparation methods for the rest of the compounds based on these exemplarily provided preparation methods.

Further, preferably, in order to obtain a compound with a higher yield and purity, the present invention provides a method described in the second aspect to prepare a compound containing a sulfamide structure, or its tautomer, mesomer, racemate, enantiomer or diastereoisomer or a mixture thereof, or a pharmaceutically acceptable salt thereof. The compound has a structure shown in formula (I),

The method comprises: performing a contact reaction between a compound shown in formula (II) and a compound shown in formula (III);

In formula (I), formula (II) and formula (III), the definitions of each group are the same as those described in the first aspect.

According to a particularly preferred specific embodiment, the compounds described in the present invention are obtained by the method shown in the following synthetic route, and the substituents involved in the synthetic route correspond to those described in the preceding text.

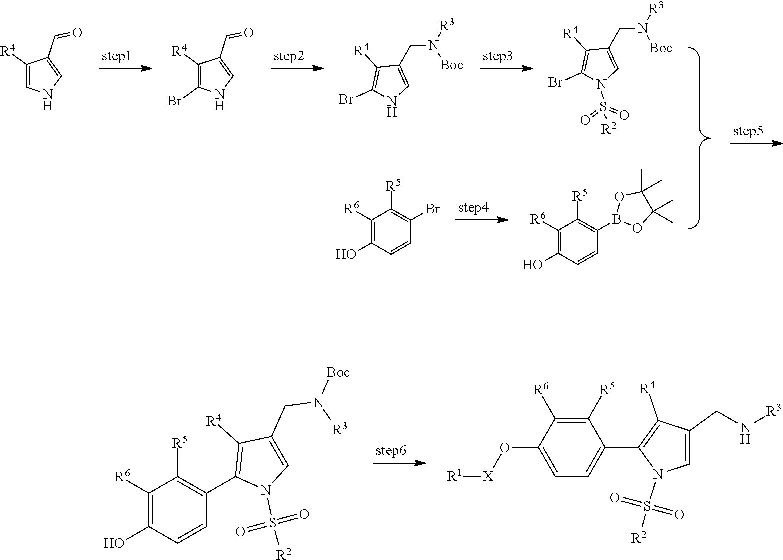

The present invention has no particular limitation to the parameter conditions in the foregoing synthetic route. Those skilled in the art may adopt the parameters known in the art. The present invention exemplarily gives the synthetic parameter conditions of some specific compounds in the subsequent text, which should not be understood by those skilled in the art as a limitation to the present invention.

As described above, the third aspect of the present invention provides a pharmaceutical composition. The pharmaceutical composition contains a therapeutically effective amount of the compound, or its tautomer, mesomer, racemate, enantiomer or diastereoisomer or a mixture thereof, or a pharmaceutically acceptable salt thereof described in the preceding first aspect. The pharmaceutical composition further contains a pharmaceutically acceptable carrier, excipient or diluent.

The present invention has no particular requirement for the pharmaceutically acceptable carrier, excipient or diluent, which can be any substance known in the art, which will not be elaborated again in the present invention and which should not be construed by those skilled in the art as a limitation to the present invention.

The fourth aspect of the present invention provides an application of the compound, or its tautomer, mesomer, racemate, enantiomer or diastereoisomer or a mixture thereof, or a pharmaceutically acceptable salt thereof involved in the preceding first aspect, or the pharmaceutical composition involved in the third aspect in the preparation of an H+/K+-ATPase inhibitor medicament and/or in the preparation of a potassium-competitive acid blocker medicament.

The fifth aspect of the present invention provides an application of the compound, or its tautomer, mesomer, racemate, enantiomer or diastereoisomer or a mixture thereof, or a pharmaceutically acceptable salt thereof involved in the preceding first aspect, or the pharmaceutical composition involved in the preceding third aspect in the preparation of a medicament for treating or preventing peptic ulcer, Zollinger Ellison Syndrome, gastritis, erosive esophagitis, reflux esophagitis, symptomatic gastroesophageal reflux disease, Barrett's esophagitis, functional dyspepsia, Helicobacter pylori infection, gastric cancer, gastric MALT lymphoma, ulcer induced by nonsteroidal anti-inflammatory drugs or post-surgical stress-induced hyperacidity or ulcer.

Particularly preferably, the peptic ulcer includes at least one of a gastric ulcer, duodenal ulcer, and anastomotic ulcer; the symptomatic gastroesophageal reflux disease includes at least one of non-erosive reflux disease and gastroesophageal reflux disease without esophagitis.

Preferably, the foregoing pharmaceutical composition and the foregoing compounds of the present invention can be used in the preparation of a gastric acid secretion inhibitor.

Preferably, the present invention provides a method for inhibiting gastric acid secretion. The method comprises giving an effective dose of the foregoing compound, or its tautomer, mesomer, racemate, enantiomer or diastereoisomer or a mixture thereof, or a pharmaceutically acceptable salt thereof to a patient in need of treatment.

In the preceding text, in addition to a pharmaceutically acceptable carrier, an excipient commonly used in pharmacology (pharmaceutics) may be included, too, such as: antibacterial agent, antifungal agent, antimicrobial agent, preservative, toner, solubilizer, thickener, surfactant, complexing agent, protein, amino acid, fat, sugar, vitamin, mineral, trace element, sweetener, pigment, flavor or a combination thereof.

The present invention will now be described in detail by means of examples. In the following examples, the raw materials used below are all ordinary commercial products, unless otherwise stated.

The present invention names the preparation examples of compound A1 to compound A15 as preparation example A1 to preparation example A15, respectively.

The present invention names the preparation examples of compound 1 to compound 100 as preparation example 1 to preparation example 100, respectively.

### Preparation Example A1: The Preparation Example is Used to Describe the Synthesis Process of the Hydrochloride Salt of Compound A1

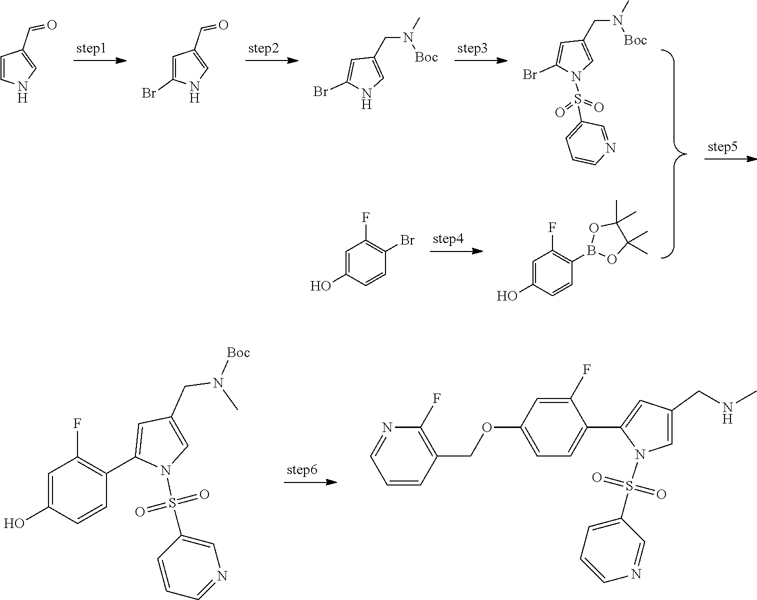

### Step 1: preparation of 5-bromo-1H-pyrrolo-3-formaldehyde

The purchased pyrrolo-3-formaldehyde (52.6 mmol) and tetrahydrofuran (THF, 100 mL) were added to a two-neck round-bottom flask and cooled in a cryogenic magnetic stirrer to −78° C., and bromo-succinimide (NBS, 52.6 mmol) dissolved in N,N-dimethylformamide (DMF, 30 mL) was added dropwise to the reaction system. After the addition, the reaction continued for 1 h, the temperature was raised to −10° C. and the reaction continued for 1 h. After it was detected by TLC that the raw materials were fully reacted, the system was added with ice water, extracted with ethyl acetate (200 mL×2) and washed with saturated brine (150 mL×2), the organic phases were merged, dried with anhydrous sodium sulfate, and concentrated under reduced pressure to obtain a brown solid, and then it was washed with isopropyl ether, and filtered to obtain a white solid, with a yield of 49.4%.

### Step 2: preparation of ((5-bromo-1H-pyrrol-3-yl)methyl)(methyl)tert-butyl carbamate

5-bromo-1H-pyrrolo-3-formaldehyde (17.2 mmol), 33% methylamine methanol solution (34.4 mmol) and methanol (40 mL) were added to a one-neck round-bottom flask and stirred at room temperature for 1 h, sodium borohydride (25.8 mmol) was added in batches in an ice bath and they were further stirred for 1 h. After it was detected by TLC that the raw materials were fully reacted, the system was added with 50 mL of water and stirred for 10 min, the reaction system was transferred with ethyl acetate (300 mL) to a separatory funnel in batches, washed with saturated brine (150 mL×3), dried with anhydrous sodium sulfate, and concentrated under reduced pressure to obtain a brownish yellow oily substance, the above product was dissolved in acetonitrile (30 mL), bi-tert-butyl dicarbonate (20.6 mmol) was added dropwise and the solution was stirred at room temperature for 30 min. After it was detected by TLC that the raw materials were fully reacted, the reaction system was transferred with ethyl acetate (300 mL) to a separatory funnel in batches, and washed with saturated brine (150 mL×3), and the organic phase was dried with anhydrous sodium sulfate, concentrated under reduced pressure, stirred, purified by column chromatography, and concentrated under reduced pressure to obtain a brown oily liquid, with a yield of 79.5%.

### Step 3: preparation of tert-butyl((5-bromo-1-(pyridin-3-ylsulfonyl)-1H-pyrrol-3-yl) methyl)(methyl)carbamate

60 mL of ultra-dry tetrahydrofuran was added to a one-neck round-bottom flask and cooled to 0° C., NaH (47.2 mmol) was added, ((5-bromo-1H-pyrrol-3-yl)methyl)(methyl)tert-butyl carbamate (13.5 mmol) dissolved in 10 mL of N,N-dimethylformamide was slowly added dropwise, they were stirred at 0° C. for 30 min, then 15-crown ether-5 (40.5 mmol) was added dropwise and then pyridine-3-sulfonyl chloride (20.2 mmol) was added dropwise. After the addition, the system was further stirred at 0° C. for 30 min and then stirred at room temperature for 30 min. After it was detected by TLC that the raw materials were fully reacted, the system was poured into ice water and extracted with ethyl acetate (200 mL×2), and washed with saturated brine (150 mL×2), the organic phases were merged, dried with anhydrous sodium sulfate, the organic phase was concentrated under reduced pressure, and purified by column chromatography to obtain a light yellow solid, with a yield of 82.1%.

### Step 4: preparation of 3-fluoro-4-(4,4,5,5-tetramethyl-1,3,2-dioxaborolan-2-yl) phenol

4-bromo-3-fluoro-phenol (262 mmol), bis(pinacolato)diboron (314 mmol), tetrakis(triphenylphosphine)palladium (13.1 mmol) and potassium acetate (524 mmol) were added to a reaction tube. 400 mL of 1,4-dioxane was added after nitrogen was pumped and charged for 3 times, and the system was stirred at 130° C. for 12 h. After it was detected by TLC that the raw materials were fully reacted, the system was added with ice water and extracted, and the organic phase was dried with anhydrous sodium sulfate, concentrated under reduced pressure and then purified by column chromatography to obtain a white solid, with a yield of 51.5%.

### Step 5: preparation of tert-butyl((5-(2-fluoro-4-hydroxyphenyl)-1-(pyridin-3-ylsulfonyl)-1H-pyrrol-3-yl) methyl)(methyl)carbamate

Tert-butyl((5-bromo-1-(pyridin-3-ylsulfonyl)-1H-pyrrol-3-yl)methyl)(methyl)carbamate (2.3 mmol), 3-fluoro-4-(4,4,5,5-tetramethyl-1,3,2-dioxaborolan-2-yl)phenol (3.49 mmol), tetrakis(triphenylphosphine)palladium (0.233 mmol) and sodium bicarbonate (6.96 mmol) were added to a reaction tube, nitrogen was pumped and charged for 3 times, DME (20 mL) and H₂O (5 mL) were added under nitrogen protection, and reaction was performed at 100° C. for 2 h. After it was detected by TLC that the raw materials were fully reacted, the reaction system was transferred with ethyl acetate (150 mL) to a separatory funnel, washed with saturated brine (100 mL×3), dried with anhydrous sodium sulfate and concentrated under reduced pressure, and the residue was purified by column chromatography to obtain a light yellow solid, with a yield of 69.8%.

### Step 6: 1-(5-(4-(benzyloxy)-2-fluorophenyl)-1-(pyridin-3-ylsulfonyl)-1H-pyrrol-3-yl)-N-methyl-methylamine hydrochloride

The stirrer was set to preheat at 80° C. Tert-butyl((5-(2-fluoro-4-hydroxyphenyl)-1-(pyridin-3-ylsulfonyl)-1H-pyrrol-3-yl)methyl)(methyl)carbamate (0.43 mmol, 1.0 eq), triphenylphosphine (0.866 mmol, 2.0 eq), 3-hydroxymethyl-2-fluoropyridine (0.866 mmol, 2.0 eq), and 10 mL of toluene were added to a one-neck round-bottom flask, heated with stirring to dissolve. When the temperature rose to 80° C., diisopropyl azodicarboxylate (0.866 mmol, 2.0 eq) was added dropwise and then stirred at 80° C. for 30 min. After it was detected by TLC that the raw materials were fully reacted, the system was directly concentrated under reduced pressure, added with 2 mL of ethyl acetate solution of hydrogen chloride, and stirred at room temperature for 12 h. After it was detected by TLC that the raw materials were fully reacted, the system was filtered to obtain hydrochloride salt of compound A1, a white solid, with a yield of 40%. ¹H NMR (600 MHz, Methanol-d₄) δ 8.85 (d, J=5.4 Hz, 1H), 8.64 (s, 1H), 8.15 (d, J=3.6 Hz, 1H), 8.11-8.02 (m, 2H), 7.76 (s, 1H), 7.74-7.68 (m, 1H), 7.37-7.28 (m, 1H), 7.04-6.96 (m, 1H), 6.82 (d, J=9.0 Hz, 1H), 6.77 (d, J=12.0 Hz, 1H), 6.38 (s, 1H), 5.16 (s, 2H), 4.03 (s, 2H), 2.64 (s, 3H).

### Preparation Example A2: The Preparation Example is Used to Describe the Synthesis Process of Hydrochloride Salt of Compound A2

The preparation method of this preparation example is similar to that of preparation example A1, with the following differences:

3-hydroxymethyl-2-fluoropyridine in step 6) was replaced with an equimolar amount of 3-hydroxymethyl-2-methylpyridine to obtain hydrochloride salt of compound A2, a yellow solid, with a one-step yield of 36% in step 6). ¹H NMR (600 MHz, Methanol-d₄) δ 8.84 (d, J=3.6 Hz, 1H), 8.70-8.61 (m, 2H), 8.57 (s, 1H), 8.10 (d, J=8.4 Hz, 1H), 7.98-7.88 (m, 1H), 7.76 (s, 1H), 7.74-7.68 (m, 1H), 7.09-7.00 (m, 1H), 6.91 (d, J=8.4 Hz, 1H), 6.87 (d, J=10.8 Hz, 1H), 6.39 (s, 1H), 5.35 (s, 2H), 4.03 (s, 2H), 2.80 (s, 3H), 2.64 (s, 3H).

### Preparation Example A3: The Preparation Example is Used to Describe the Synthesis Process of Hydrochloride Salt of Compound A3

The preparation method of this preparation example is similar to that of preparation example A1, with the following differences:

3-hydroxymethyl-2-fluoropyridine in step 6) was replaced with an equimolar amount of 4-hydroxymethyl-1-methyl-pyrazole to obtain hydrochloride salt of compound A3, a white solid, with a one-step yield of 24% in step 6). ¹H NMR (600 MHz, Methanol-d₄) δ 8.79 (s, 1H), 8.51 (s, 1H), 7.92-7.69 (m, 3H), 7.62-7.44 (m, 2H), 6.99 (s, 1H), 6.86-6.62 (m, 4H), 6.38 (s, 1H), 5.05 (s, 2H), 4.08 (s, 2H), 3.90 (s, 3H), 2.70 (s, 3H).

### Preparation Example A4: The Preparation Example is Used to Describe the Synthesis Process of Hydrochloride Salt of Compound A4

The preparation method of this preparation example is similar to that of preparation example A1, with the following differences:

3-hydroxymethyl-2-fluoropyridine in step 6) was replaced with an equimolar amount of 5-hydroxymethyl-2-methoxypyridine to obtain hydrochloride salt of compound A4, a yellow solid, with a one-step yield of 45% in step 6). ¹H NMR (600 MHz, Methanol-d₄) δ 8.76 (d, J=5.4 Hz, 1H), 8.50 (d, J=2.4 Hz, 1H), 8.21 (d, J=2.4 Hz, 1H), 7.83 (d, J=8.4 Hz, 1H), 7.79 (dd, J=8.4, 2.4 Hz, 1H), 7.67 (s, 1H), 7.49 (dd, J=8.4, 4.8 Hz, 1H), 6.99 (t, J=8.4 Hz, 1H), 6.86-6.79 (m, 2H), 6.74 (dd, J=11.4, 2.4 Hz, 1H), 6.33 (s, 1H), 5.07 (s, 2H), 3.92 (s, 2H), 3.90 (s, 3H), 2.58 (s, 3H).

### Preparation Example A5: The Preparation Example is Used to Describe the Synthesis Process of Hydrochloride Salt of Compound A5

The preparation method of this preparation example is similar to that of preparation example A1, with the following differences:

3-hydroxymethyl-2-fluoropyridine in step 6) was replaced with an equimolar amount of 2-fluoro-6-hydroxymethylpyridine to obtain hydrochloride salt of compound A5, a white solid, with a one-step yield of 46% in step 6). ¹H NMR (600 MHz, Methanol-d₄) δ 8.85 (s, 1H), 8.63 (s, 1H), 8.05 (d, J=8.4 Hz, 1H), 7.98-7.90 (m, 1H), 7.77 (d, J=1.8 Hz, 1H), 7.71-7.64 (m, 1H), 7.46 (d, J=7.8 Hz, 1H), 7.02-6.93 (m, 2H), 6.83 (dd, J=8.4, 2.4 Hz, 1H), 6.78-6.70 (m, 1H), 6.38 (d, J=1.8 Hz, 1H), 5.14 (s, 2H), 4.05 (s, 2H), 2.66 (s, 3H).

### Preparation Example A6: The Preparation Example is Used to Describe the Synthesis Process of Hydrochloride Salt of Compound A6

The preparation method of this preparation example is similar to that of preparation example A1, with the following differences:

3-hydroxymethyl-2-fluoropyridine in step 6) was replaced with an equimolar amount of 3-fluoro-2-hydroxymethylpyridine to obtain hydrochloride salt of compound A6, a yellow solid, with a one-step yield of 49% in step 6). ¹H NMR (600 MHz, Methanol-d₄) δ 8.82 (d, J=4.8 Hz, 1H), 8.70 (d, J=5.4 Hz, 1H), 8.58 (s, 1H), 8.43 (t, J=8.4 Hz, 1H), 8.10 (d, J=7.8 Hz, 1H), 8.05-8.00 (m, 1H), 7.75-7.68 (m, 2H), 7.04 (t, J=8.4 Hz, 1H), 6.97-6.87 (m, 2H), 6.39-6.34 (m, 1H), 5.55 (s, 2H), 3.99 (s, 2H), 2.59 (s, 3H).

### Preparation Example A7: The Preparation Example is Used to Describe the Synthesis Process of Hydrochloride Salt of Compound A7

The preparation method of this preparation example is similar to that of preparation example A1, with the following differences:

3-hydroxymethyl-2-fluoropyridine in step 6) was replaced with an equimolar amount of 3-hydroxymethylpyridazine to obtain hydrochloride salt of compound A7, a yellow solid, with a one-step yield of 41% in step 6). ¹H NMR (600 MHz, Methanol-d₄) δ 9.68-9.59 (m, 1H), 8.85 (s, 1H), 8.76 (d, J=8.4 Hz, 1H), 8.66-8.50 (m, 2H), 8.03 (d, J=8.4 Hz, 1H), 7.82 (s, 1H), 7.65 (t, J=6.6 Hz, 1H), 7.11 (t, J=8.4 Hz, 1H), 7.03-6.88 (m, 2H), 6.45 (s, 1H), 5.66 (s, 2H), 4.10 (s, 2H), 2.71 (s, 3H).

### Preparation Example A8: The Preparation Example is Used to Describe the Synthesis Process of Hydrochloride Salt of Compound A8

The preparation method of this preparation example is similar to that of preparation example A1, with the following differences:

3-hydroxymethyl-2-fluoropyridine in step 6) was replaced with an equimolar amount of 3-hydroxymethyl-1-methylpyrazole to obtain hydrochloride salt of compound A8, a white solid, with a one-step yield of 38% in step 6). ¹H NMR (600 MHz, Methanol-d₄) δ 8.85 (s, 1H), 8.58 (s, 1H), 7.98 (d, J=6.0 Hz, 1H), 7.84-7.78 (m, 2H), 7.66-7.60 (m, 1H), 7.04 (t, J=8.4 Hz, 1H), 6.90-6.85 (m, 1H), 6.79 (d, J=11.4 Hz, 1H), 6.54 (s, 1H), 6.41 (s, 1H), 5.20 (s, 2H), 4.10 (s, 2H), 4.00 (s, 3H), 2.71 (s, 3H).

### Preparation Example A9: The Preparation Example is Used to Describe the Synthesis Process of Hydrochloride Salt of Compound A9

The preparation method of this preparation example is similar to that of preparation example A1, with the following differences:

3-hydroxymethyl-2-fluoropyridine in step 6) was replaced with an equimolar amount of cyclopropylmethanol to obtain hydrochloride salt of compound A9, a white solid, with a one-step yield of 48% in step 6). ¹H NMR (600 MHz, Chloroform-d) δ 8.77-8.71 (m, 1H), 8.62 (d, J=2.4 Hz, 1H), 7.72 (dt, J=8.4, 1.8 Hz, 1H), 7.40 (s, 1H), 7.33 (dd, J=8.4, 4.8 Hz, 1H), 7.02 (t, J=8.4 Hz, 1H), 6.66 (dd, J=8.4, 2.4 Hz, 1H), 6.56 (dd, J=11.4, 2.4 Hz, 1H), 6.23 (d, J=1.8 Hz, 1H), 3.81 (d, J=7.2 Hz, 2H), 3.66 (s, 2H), 2.45 (s, 3H), 1.32-1.28 (m, 1H), 0.72-0.62 (m, 2H), 0.40-0.33 (m, 2H).

### Preparation Example A10: The Preparation Example is Used to Describe the Synthesis Process of Hydrochloride Salt of Compound A10

The preparation method of this preparation example is similar to that of preparation example A1, with the following differences:

3-hydroxymethyl-2-fluoropyridine in step 6) was replaced with an equimolar amount of (2, 2-difluorocyclopropyl)methanol to obtain hydrochloride salt of compound A10, a white solid, with a one-step yield of 36% in step 6). ¹H NMR (600 MHz, Methanol-d₄) δ 8.90 (d, J=4.8 Hz, 1H), 8.68 (s, 1H), 8.12 (d, J=7.8 Hz, 1H), 7.82 (s, 1H), 7.75 (dd, J=8.4, 4.8 Hz, 1H), 7.03 (t, J=8.4 Hz, 1H), 6.84-6.77 (m, 1H), 6.75-6.68 (m, 1H), 6.43 (s, 1H), 4.26-4.18 (m, 1H), 4.10 (s, 2H), 4.07-4.00 (m, 1H), 2.71 (s, 3H), 2.23-2.11 (m, 1H), 1.71-1.61 (m, 1H), 1.44-1.34 (m, 1H).

### Preparation Example A11

The preparation example is used to describe the synthesis process of hydrochloride salt of compound A11:

The preparation method of this preparation example is similar to that of preparation example A1, with the following differences:

3-hydroxymethyl-2-fluoropyridine in step 6) was replaced with an equimolar amount of ethylene oxide-2-ylmethanol to obtain hydrochloride salt of compound A11, a white solid, with a one-step yield of 60% in step 6). ¹H NMR (600 MHz, Methanol-d₄) δ 8.91 (d, J=4.8 Hz, 1H), 8.68 (s, 1H), 8.12 (d, J=8.4 Hz, 1H), 7.82 (d, J=2.4 Hz, 1H), 7.76 (dd, J=8.4, 4.8 Hz, 1H), 7.04 (t, J=8.4 Hz, 1H), 6.85-6.79 (m, 1H), 6.74 (dd, J=11.4, 3.0 Hz, 1H), 6.43 (t, J=3.0 Hz, 1H), 4.20-4.11 (m, 2H), 4.10 (s, 2H), 3.82-3.76 (m, 1H), 3.73-3.68 (m, 1H), 2.71 (s, 3H).

### Preparation Example A12: The Preparation Example is Used to Describe the Synthesis Process of Hydrochloride Salt of Compound A12

The preparation method of this preparation example is similar to that of preparation example A1, with the following differences:

3-hydroxymethyl-2-fluoropyridine in step 6) was replaced with an equimolar amount of oxetan-3-ylmethanol to obtain hydrochloride salt of compound A12, a white solid, with a one-step yield of 44% in step 6). ¹H NMR (600 MHz, Chloroform-d) δ 8.69 (d, J=4.8 Hz, 1H), 8.55 (d, J=2.4 Hz, 1H), 7.69 (d, J=8.4 Hz, 1H), 7.36 (s, 1H), 7.28 (dd, J=8.4, 4.8 Hz, 1H), 6.99 (t, J=8.4 Hz, 1H), 6.63 (dd, J=8.4, 2.4 Hz, 1H), 6.54 (dd, J=11.4, 2.4 Hz, 1H), 6.24-6.17 (m, 1H), 4.88-4.80 (m, 2H), 4.56-4.48 (m, 2H), 4.15 (d, J=6.6 Hz, 2H), 3.61 (s, 2H), 3.45-3.36 (m, 1H), 2.41 (s, 3H).

### Preparation Example A13: The Preparation Example is Used to Describe the Synthesis Process of Hydrochloride Salt of Compound A13

The preparation method of this preparation example is similar to that of preparation example A1, with the following differences:

3-hydroxymethyl-2-fluoropyridine in step 6) was replaced with an equimolar amount of tetrahydrofuran-3-ylmethanol to obtain hydrochloride salt of compound A13, a white solid, with a one-step yield of 40% in step 6). ¹H NMR (400 MHz, Methanol-d₄) δ 8.81 (d, J=5.2 Hz, 1H), 8.56 (d, J=2.4 Hz, 1H), 7.91 (d, J=8.4 Hz, 1H), 7.78 (s, 1H), 7.57 (dd, J=8.4, 4.8 Hz, 1H), 7.00 (t, J=8.4 Hz, 1H), 6.77 (d, J=8.8 Hz, 1H), 6.69 (d, J=12.8 Hz, 1H), 6.38 (d, J=2.0 Hz, 1H), 4.09 (s, 2H), 4.06-4.00 (m, 1H), 3.99-3.96 (m, 1H), 3.95-3.88 (m, 2H), 3.83-3.75 (m, 1H), 3.74-3.68 (m, 1H), 2.82-2.73 (m, 1H), 2.71 (s, 3H), 2.21-2.10 (m, 1H), 1.85-1.73 (m, 1H).

### Preparation Example A14: The Preparation Example is Used to Describe the Synthesis Process of Hydrochloride Salt of Compound A14

The preparation method of this preparation example is similar to that of preparation example A1, with the following differences:

3-hydroxymethyl-2-fluoropyridine in step 6) was replaced with an equimolar amount of tetrahydrofuran-2-ylmethanol to obtain hydrochloride salt of compound A14, a white solid, with a one-step yield of 46% in step 6). ¹H NMR (600 MHz, Methanol-d₄) δ 8.92 (d, J=5.4 Hz, 1H), 8.72-8.65 (m, 1H), 8.14 (d, J=8.4 Hz, 1H), 7.86-7.75 (m, 2H), 7.02 (t, J=8.4 Hz, 1H), 6.85-6.77 (m, 1H), 6.75-6.66 (m, 1H), 6.45-6.36 (m, 1H), 4.34-4.25 (m, 1H), 4.14-4.05 (m, 3H), 4.03-3.98 (m, 1H), 3.97-3.89 (m, 1H), 3.88-3.81 (m, 1H), 2.71 (s, 3H), 2.17-2.08 (m, 1H), 2.06-1.92 (m, 2H), 1.87-1.75 (m, 1H).

### Preparation Example A15: The Preparation Example is Used to Describe the Synthesis Process of Hydrochloride Salt of Compound A15

The preparation method of this preparation example is similar to that of preparation example A1, with the following differences:

3-hydroxymethyl-2-fluoropyridine in step 6) was replaced with an equimolar amount of oxetan-2-ylmethanol to obtain hydrochloride salt of compound A15, a yellow solid, with a one-step yield of 52% in step 6). ¹H NMR (600 MHz, Methanol-d₄) δ 8.95 (d, J=5.4 Hz, 1H), 8.73 (t, J=4.2 Hz, 1H), 8.21 (d, J=8.4 Hz, 1H), 7.89-7.78 (m, 2H), 7.05 (t, J=8.4 Hz, 1H), 6.87-6.80 (m, 1H), 6.77-6.70 (m, 1H), 6.45 (d, J=7.2 Hz, 1H), 4.22-4.15 (m, 1H), 4.15-4.09 (m, 2H), 4.08-3.96 (m, 2H), 3.79 (s, 2H), 2.73 (s, 3H), 2.13-1.99 (m, 2H).

### Preparation Example 1: The Preparation Example is Used to Describe the Synthesis Process of Hydrochloride Salt of Compound 1

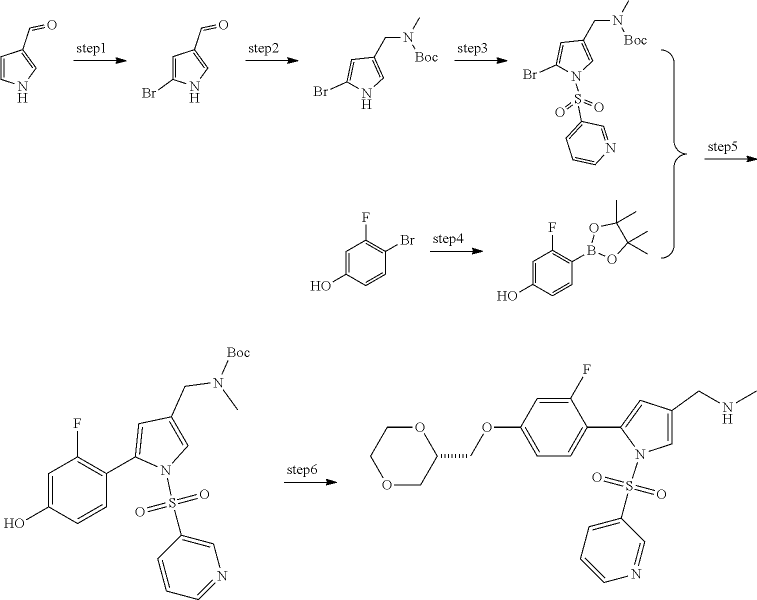

### Step 1: preparation of 5-bromo-1H-pyrrolo-3-formaldehyde

Pyrrolo-3-formaldehyde (52.6 mmol) and tetrahydrofuran (THF, 100 mL) were added to a two-neck round-bottom flask and cooled in a cryogenic magnetic stirrer to −78° C., and bromo-succinimide (NBS, 52.6 mmol) dissolved in N,N-dimethylformamide (DMF, 30 mL) was added dropwise to the reaction system. After the addition, the reaction continued for 1 h, the temperature was raised to −10° C. and the reaction continued for 1 h. After it was detected by TLC that the raw materials were fully reacted, the system was added with ice water, extracted with ethyl acetate (200 mL×2) and washed with saturated brine (150 mL×2), the organic phases were merged, dried with anhydrous sodium sulfate, and concentrated under reduced pressure to obtain a brown solid, and then it was washed with isopropyl ether, and filtered to obtain a white solid, with a yield of 49.4%.

### Step 2: preparation of ((5-bromo-1H-pyrrol-3-yl)methyl)(methyl)tert-butyl carbamate

5-bromo-1H-pyrrolo-3-formaldehyde (17.2 mmol), 33 wt % methylamine methanol solution (34.4 mmol) and methanol (40 mL) were added to a one-neck round-bottom flask and stirred at room temperature (25° C., the same below) for 1 h, sodium borohydride (25.8 mmol) was added in batches in an ice bath and they were further stirred for 1 h. After it was detected by TLC that the raw materials were fully reacted, the system was added with 50 mL of water and stirred for 10 min, the reaction system was transferred with ethyl acetate (300 mL) to a separatory funnel in batches, and washed with saturated brine (150 mL×3), dried with anhydrous sodium sulfate, and concentrated under reduced pressure to obtain a brownish yellow oily substance, the above product was dissolved in acetonitrile (30 mL), bi-tert-butyl dicarbonate (20.6 mmol) was added dropwise and the solution was stirred at room temperature for 30 min. After it was detected by TLC that the raw materials were fully reacted, the reaction system was transferred with ethyl acetate (300 mL) to a separatory funnel in batches, and washed with saturated brine (150 mL×3), and the organic phase was dried with anhydrous sodium sulfate, concentrated under reduced pressure, stirred, purified by column chromatography, and concentrated under reduced pressure to obtain a brown oily liquid, with a yield of 79.5%.

### Step 3: preparation of tert-butyl((5-bromo-1-(pyridin-3-ylsulfonyl)-1H-pyrrol-3-yl) methyl)(methyl)carbamate

60 mL of ultra-dry tetrahydrofuran was added to a one-neck round-bottom flask and cooled to 0° C., NaH (47.2 mmol) was added, ((5-bromo-1H-pyrrol-3-yl)methyl)(methyl)tert-butyl carbamate (13.5 mmol) dissolved in 10 mL of N,N-dimethylformamide was slowly added dropwise, they were stirred at 0° C. for 30 min, then 15-crown ether-5 (40.5 mmol) was added dropwise and then pyridine-3-sulfonyl chloride (20.2 mmol) was added dropwise. After the addition, the system was further stirred at 0° C. for 30 min and then stirred at room temperature for 30 min. After it was detected by TLC that the raw materials were fully reacted, the system was poured into ice water and extracted with ethyl acetate (200 mL×2), and washed with saturated brine (150 mL×2), the organic phases were merged, dried with anhydrous sodium sulfate, the organic phase was concentrated under reduced pressure, and purified by column chromatography to obtain a light yellow solid, with a yield of 82.1%.

### Step 4: preparation of 3-fluoro-4-(4,4,5,5-tetramethyl-1,3,2-dioxaborolan-2-yl) phenol

4-bromo-3-fluoro-phenol (262 mmol), bis(pinacolato)diboron (314 mmol), tetrakis(triphenylphosphine)palladium (13.1 mmol) and potassium acetate (524 mmol) were added to a reaction tube. 400 mL of 1,4-dioxane was added after nitrogen was pumped and charged for 3 times, and the system was stirred at 130° C. for 12 h. After it was detected by TLC that the raw materials were fully reacted, the system was added with ice water and extracted, and the organic phase was dried with anhydrous sodium sulfate, concentrated under reduced pressure and then purified by column chromatography to obtain a white solid, with a yield of 51.5%.

### Step 5: preparation of tert-butyl((5-(2-fluoro-4-hydroxyphenyl)-1-(pyridin-3-ylsulfonyl)-1H-pyrrol-3-yl)methyl)(methyl)carbamate

Tert-butyl((5-bromo-1-(pyridin-3-ylsulfonyl)-1H-pyrrol-3-yl)methyl)(methyl)carbamate (2.3 mmol), 3-fluoro-4-(4,4,5,5-tetramethyl-1,3,2-dioxaborolan-2-yl)phenol (3.49 mmol), tetrakis(triphenylphosphine)palladium (0.233 mmol) and sodium bicarbonate (6.96 mmol) were added to a reaction tube, nitrogen was pumped and charged for 3 times, DME (20 mL) and H₂O (5 mL) were added under nitrogen protection, and reaction was performed at 100° C. for 2 h. After it was detected by TLC that the raw materials were fully reacted, the reaction system was transferred with ethyl acetate (150 mL) to a separatory funnel, washed with saturated brine (100 mL×3), dried with anhydrous sodium sulfate, concentrated under reduced pressure, and the residue was purified by column chromatography to obtain a light yellow solid, with a yield of 69.8%.

### Step 6: (S)-1-(5-(4-((1,4-dioxane-2-yl)methoxy)-2-fluorophenyl)-1-(pyridine-3-ylsulfonyl)-1H-pyrrole-3-yl)-N-methyl methylamine hydrochloride

The stirrer was set to preheat at 80° C. Tert-butyl((5-(2-fluoro-4-hydroxyphenyl)-1-(pyridin-3-ylsulfonyl)-1H-pyrrol-3-yl)methyl)(methyl)carbamate (0.43 mmol, 1.0 eq), triphenylphosphine (0.866 mmol, 2.0 eq), (2S)-1,4-dioxane-2-methanol (0.866 mmol, 2.0 eq) and 10 mL of toluene were added to a one-neck round-bottom flask, heated with stirring to dissolve. When the temperature rose to 80° C., diisopropyl azodicarboxylate (0.866 mmol, 2.0 eq) was added dropwise and stirred at 80° C. for 30 min. After it was detected by TLC that the raw materials were fully reacted, the system was directly concentrated under reduced pressure, added with 2 mL of ethyl acetate solution of hydrogen chloride, and stirred at room temperature for 12 h. After it was detected by TLC that the raw materials were fully reacted, the system was filtered to obtain hydrochloride salt of compound 1, a yellow solid, with a yield of 50%.

¹H NMR (600 MHz, Methanol-d₄) δ 8.88 (d, J=4.8 Hz, 1H), 8.65 (s, 1H), 8.07 (d, J=8.4 Hz, 1H), 7.81 (s, 1H), 7.75-7.69 (m, 1H), 7.02 (t, J=8.4 Hz, 1H), 6.79 (d, J=8.4 Hz, 1H), 6.72 (d, J=11.4 Hz, 1H), 6.42 (s, 1H), 4.10 (s, 2H), 4.08-4.01 (m, 2H), 4.00-3.95 (m, 1H), 3.92-3.88 (m, 1H), 3.86-3.82 (m, 1H), 3.81-3.71 (m, 2H), 3.67-3.61 (m, 1H), 3.58-3.53 (m, 1H), 2.71 (s, 3H).

### Preparation Example 2: The Preparation Example is Used to Describe the Synthesis Process of Hydrochloride Salt of Compound 2

The preparation method of this preparation example is similar to that of preparation example 1, with the following differences:

(2S)-1,4-dioxane-2-methanol in step 6) was replaced with an equimolar amount of cyclobutylmethanol to obtain hydrochloride salt of compound 2, a white solid, with a one-step yield of 24% in step 6).

¹H NMR (600 MHz, Methanol-d₄) δ 8.87-8.83 (m, 1H), 8.61 (s, 1H), 8.01 (d, J=8.4 Hz, 1H), 7.80 (s, 1H), 7.68-7.63 (m, 1H), 7.00 (t, J=8.4 Hz, 1H), 6.76 (d, J=8.4 Hz, 1H), 6.69-6.62 (m, 1H), 6.40 (d, J=1.8 Hz, 1H), 4.10 (s, 2H), 4.00 (d, J=6.6 Hz, 2H), 2.86-2.77 (m, 1H), 2.72 (s, 3H), 2.23-2.11 (m, 2H), 2.08-1.89 (m, 4H).

### Preparation Example 3: The Preparation Example is Used to Describe the Synthesis Process of Hydrochloride Salt of Compound 3

The preparation method of this preparation example is similar to that of preparation example 1, with the following differences:

(2S)-1,4-dioxane-2-methanol in step 6) was replaced with an equimolar amount of cyclopentylmethanol to obtain hydrochloride salt of compound 3, a white solid, with a one-step yield of 31% in step 6).

¹H NMR (600 MHz, Methanol-d₄) δ 8.87 (d, J=4.8 Hz, 1H), 8.63 (s, 1H), 8.05 (d, J=8.4 Hz, 1H), 7.81 (d, J=2.4 Hz, 1H), 7.72-7.66 (m, 1H), 7.00 (t, J=8.4 Hz, 1H), 6.76 (d, J=8.4 Hz, 1H), 6.67 (d, J=11.4 Hz, 1H), 6.41 (d, J=1.8 Hz, 1H), 4.10 (s, 2H), 3.92 (d, J=6.6 Hz, 2H), 2.72 (s, 3H), 2.44-2.32 (m, 1H), 1.92-1.83 (m, 2H), 1.75-1.58 (m, 4H), 1.47-1.35 (m, 2H).

### Preparation Example 4: The Preparation Example is Used to Describe the Synthesis Process of Hydrochloride Salt of Compound 4

The preparation method of this preparation example is similar to that of preparation example 1, with the following differences: (2S)-1,4-dioxane-2-methanol in step 6) was replaced with an equimolar amount of cyclohexylmethanol to obtain hydrochloride salt of compound 4, a white solid, with a one-step yield of 35% in step 6).

¹H NMR (600 MHz, Methanol-d₄) δ 8.84 (d, J=5.4 Hz, 1H), 8.60 (s, 1H), 8.06-7.99 (m, 1H), 7.76 (d, J=2.4 Hz, 1H), 7.68-7.62 (m, 1H), 6.97-6.90 (m, 1H), 6.70 (dd, J=8.4, 2.4 Hz, 1H), 6.63-6.57 (m, 1H), 6.36 (d, J=2.4 Hz, 1H), 4.04 (s, 2H), 3.78 (d, J=6.6 Hz, 2H), 2.66 (s, 3H), 1.88-1.62 (m, 6H), 1.35-1.15 (m, 3H), 1.12-0.98 (m, 2H).

### Preparation Example 5: The Preparation Example is Used to Describe the Synthesis Process of Hydrochloride Salt of Compound 5

The preparation method of this preparation example is similar to that of preparation example 1, with the following differences:

(2S)-1,4-dioxane-2-methanol in step 6) was replaced with an equimolar amount of 4,4-difluoro-cyclohexanemethanol to obtain hydrochloride salt of compound 5, a white solid, with a one-step yield of 37% in step 6).

¹H NMR (600 MHz, Methanol-d₄) δ 8.85 (dd, J=4.8, 1.8 Hz, 1H), 8.60 (d, J=2.4 Hz, 1H), 8.00 (dt, J=8.4, 1.8 Hz, 1H), 7.79 (d, J=1.8 Hz, 1H), 7.65 (dd, J=8.4, 4.8 Hz, 1H), 7.00 (t, J=8.4 Hz, 1H), 6.76 (dd, J=8.4, 2.4 Hz, 1H), 6.68 (dd, J=11.4, 2.4 Hz, 1H), 6.40 (d, J=1.8 Hz, 1H), 4.09 (s, 2H), 3.91 (d, J=6.0 Hz, 2H), 2.71 (s, 3H), 2.15-2.05 (m, 2H), 2.01-1.92 (m, 3H), 1.91-1.75 (m, 2H), 1.51-1.40 (m, 2H).

### Preparation Example 6

The preparation example is used to describe the synthesis process of compound 6:

The preparation method of this preparation example is similar to that of preparation example 1, with the following differences: (2S)-1,4-dioxane-2-methanol in step 6) was replaced with an equimolar amount of 4-hydroxymethyltetrahydropyran to obtain compound 6, a white solid, with a one-step yield of 34% in step 6).

¹H NMR (400 MHz, DMSO-d₆) δ 8.86 (dd, J=4.8, 1.8 Hz, 1H), 8.57 (d, J=2.4 Hz, 1H), 7.88 (dt, J=8.4, 1.8 Hz, 1H), 7.61 (dd, J=8.4, 4.8 Hz, 1H), 7.45 (d, J=1.8 Hz, 1H), 7.00 (t, J=8.4 Hz, 1H), 6.85 (dd, J=12.0, 2.4 Hz, 1H), 6.78 (dd, J=8.4, 2.4 Hz, 1H), 6.30 (d, J=1.8 Hz, 1H), 3.90 (dd, J=11.4, 4.8 Hz, 4H), 3.49 (s, 2H), 2.24 (s, 3H), 2.10-1.96 (m, 1H), 1.75-1.63 (m, 2H), 1.44-1.20 (m, 4H).

### Preparation Example 7: The Preparation Example is Used to Describe the Synthesis Process of Hydrochloride Salt of Compound 7

The preparation method of this preparation example is similar to that of preparation example 1, with the following differences:

(2S)-1,4-dioxane-2-methanol in step 6) was replaced with an equimolar amount of tetrahydropyran-2-methanol to obtain hydrochloride salt of compound 7, a white solid, with a one-step yield of 40% in step 6).

¹H NMR (600 MHz, Methanol-d₄) δ 8.89 (dd, J=4.8, 1.8 Hz, 1H), 8.65 (d, J=2.4 Hz, 1H), 8.07 (d, J=8.4 Hz, 1H), 7.81 (d, J=1.8 Hz, 1H), 7.72 (dd, J=8.4, 4.8 Hz, 1H), 7.01 (t, J=8.4 Hz, 1H), 6.79 (dd, J=8.4, 2.54 Hz, 1H), 6.70 (dd, J=11.4, 2.4 Hz, 1H), 6.42 (d, J=1.8 Hz, 1H), 4.10 (s, 2H), 4.04-3.97 (m, 3H), 3.79-3.72 (m, 1H), 3.58-3.50 (m, 1H), 2.71 (s, 3H), 1.96-1.88 (m, 1H), 1.76-1.43 (m, 5H).

### Preparation Example 8: The Preparation Example is Used to Describe the Synthesis Process of Hydrochloride Salt of Compound 8

The preparation method of this preparation example is similar to that of preparation example 1, with the following differences:

(2S)-1,4-dioxane-2-methanol in step 6) was replaced with an equimolar amount of 1-(tetrahydro-2H-pyran-4-yl)ethanol to obtain hydrochloride salt of compound 8, a white solid, with a one-step yield of 25% in step 6).

¹H NMR (600 MHz, Methanol-d₄) δ 8.86 (dd, J=4.8, 1.6 Hz, 1H), 8.62 (d, J=2.4 Hz, 1H), 8.04-7.98 (m, 1H), 7.81 (d, J=1.8 Hz, 1H), 7.65 (dd, J=8.4, 4.8 Hz, 1H), 7.01 (t, J=8.4 Hz, 1H), 6.76 (dd, J=8.4, 2.4 Hz, 1H), 6.67 (dd, J=12.0, 2.4 Hz, 1H), 6.41 (d, J=1.8 Hz, 1H), 4.35-4.29 (m, 1H), 4.10 (s, 2H), 4.01 (dd, J=11.8, 4.2 Hz, 2H), 3.50-3.40 (m, 2H), 2.72 (s, 3H), 1.92-1.80 (m, 2H), 1.70-1.62 (m, 1H), 1.58-1.42 (m, 2H), 1.32 (d, J=6.0 Hz, 3H).

### Preparation Example 9: The Preparation Example is Used to Describe the Synthesis Process of Hydrochloride Salt of Compound 9

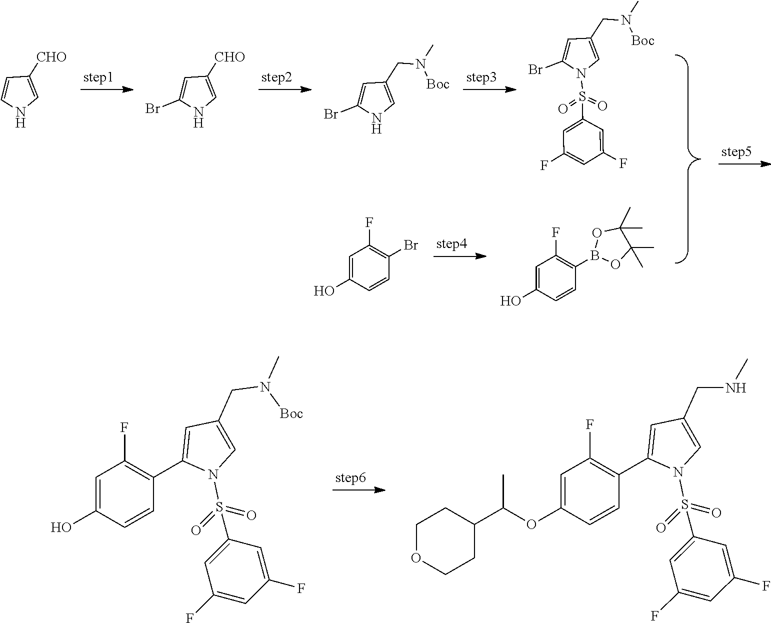

The preparation method of this preparation example is similar to that of preparation example 1, with the following differences: pyridine-3-sulfonyl chloride in step 3) was replaced with an equimolar amount of 3,5-difluorobenzenesulfonyl chloride, a yellow solid, with a yield of 70%.

Tert-butyl((5-bromo-1-(pyridin-3-ylsulfonyl)-1H-pyrrol-3-yl)methyl)(methyl)carbamate in step 5) was replaced with an equimolar amount of tert-butyl((5-bromo-1-(3,5-difluorobenzenesulfonyl)-1H-pyrrol-3-yl)methyl)(methyl)carbamate, a yellow solid, with a yield of 61%.

(2S)-1,4-dioxane-2-methanol in step 6) was replaced with an equimolar amount of 1-(tetrahydro-2H-pyran-4-yl)ethanol to obtain hydrochloride salt of compound 9, a white solid, with a one-step yield of 28% in step 6).

¹H NMR (600 MHz, Methanol-d₄) δ 7.74 (d, J=11.4 Hz, 1H), 7.38 (t, J=9.0 Hz, 1H), 7.09-6.95 (m, 3H), 6.75 (d, J=8.4 Hz, 1H), 6.66 (d, J=11.4 Hz, 1H), 6.40 (s, 1H), 4.36-4.28 (m, 1H), 4.09 (s, 2H), 4.02-3.92 (m, 2H), 3.50-3.38 (m, 2H), 2.71 (s, 3H), 1.92-1.79 (m, 2H), 1.69-1.39 (m, 3H), 1.30 (d, J=6.0 Hz, 3H).

### Preparation Example 10: The Preparation Example is Used to Describe the Synthesis Process of Hydrochloride Salt of Compound 10

The preparation method of this preparation example is similar to that of preparation example 1, with the following differences:

Pyridine-3-sulfonyl chloride in step 3) was replaced with an equimolar amount of 3-trifluoromethylbenzenesulfonyl chloride, a yellow solid, with a yield of 70%.

Tert-butyl((5-bromo-1-(pyridin-3-ylsulfonyl)-1H-pyrrol-3-yl)methyl)(methyl)carbamate in step 5) was replaced with an equimolar amount of tert-butyl((5-bromo-1-(3-trifluoromethylbenzenesulfonyl)-1H-pyrrol-3-yl)methyl)(methyl)carbamate, a yellow solid, with a yield of 60%.

(2S)-1,4-dioxane-2-methanol in step 6) was replaced with an equimolar amount of 1-(tetrahydro-2H-pyran-4-yl)ethanol to obtain hydrochloride salt of compound 10, a white solid, with a one-step yield of 35% in step 6).

¹H NMR (600 MHz, Methanol-d₄) δ 8.03 (d, J=7.8 Hz, 1H), 7.89-7.72 (m, 3H), 7.60 (s, 1H), 6.99 (t, J=8.4 Hz, 1H), 6.75 (d, J=8.4 Hz, 1H), 6.64 (d, J=11.4 Hz, 1H), 6.39 (d, J=2.4 Hz, 1H), 4.35-4.29 (m, 1H), 4.11 (s, 2H), 4.05-3.99 (m, 2H), 3.51-3.44 (m, 2H), 2.72 (s, 3H), 1.95-1.85 (m, 2H), 1.71-1.65 (m, 1H), 1.60-1.45 (m, 2H), 1.33 (d, J=6.6 Hz, 3H).

### Preparation Example 11: The Preparation Example is Used to Describe the Synthesis Process of Hydrochloride Salt of Compound 11

The preparation method of this preparation example is similar to that of preparation example 1, with the following differences:

Pyridine-3-sulfonyl chloride in step 3) was replaced with an equimolar amount of 3-fluoro-4-methylbenzenesulfonyl chloride, a yellow solid, with a yield of 73%.

Tert-butyl((5-bromo-1-(pyridin-3-ylsulfonyl)-1H-pyrrol-3-yl)methyl)(methyl)carbamate in step 5) was replaced with an equimolar amount of tert-butyl((5-bromo-1-(3-fluoro-4-methylbenzenesulfonyl)-1H-pyrrol-3-yl)methyl)(methyl)carbamate, a yellow solid, with a yield of 63%.

(2S)-1,4-dioxane-2-methanol in step 6) was replaced with an equimolar amount of 1-(tetrahydro-2H-pyran-4-yl)ethanol to obtain hydrochloride salt of compound 11, a white solid, with a one-step yield of 31% in step 6).

¹H NMR (600 MHz, Methanol-d₄) δ 7.75 (s, 1H), 7.40 (t, J=7.8 Hz, 1H), 7.24 (d, J=8.4 Hz, 1H), 7.06 (d, J=8.4 Hz, 1H), 7.00 (t, J=8.4 Hz, 1H), 6.75 (d, J=8.4 Hz, 1H), 6.69-6.64 (m, 1H), 6.38 (s, 1H), 4.37-4.30 (m, 1H), 4.10 (s, 2H), 4.04-3.99 (m, 2H), 3.47 (t, J=12.0 Hz, 2H), 2.72 (s, 3H), 2.34 (s, 3H), 1.96-1.83 (m, 2H), 1.71-1.63 (m, 1H), 1.57-1.43 (m, 2H), 1.33 (d, J=6.0 Hz, 3H).

### Preparation Example 12: The Preparation Example is Used to Describe the Synthesis Process of Hydrochloride Salt of Compound 12

The preparation method of this preparation example is similar to that of preparation example 1, with the following differences:

Pyridine-3-sulfonyl chloride in step 3) was replaced with an equimolar amount of 2,5-difluorobenzenesulfonyl chloride, a yellow solid, with a yield of 72%.

Tert-butyl((5-bromo-1-(pyridin-3-ylsulfonyl)-1H-pyrrol-3-yl)methyl)(methyl)carbamate in step 5) was replaced with an equimolar amount of tert-butyl((5-bromo-1-(2,5-difluorobenzenesulfonyl)-1H-pyrrol-3-yl)methyl)(methyl)carbamate, a yellow solid, with a yield of 61%.

(2S)-1,4-dioxane-2-methanol in step 6) was replaced with an equimolar amount of 1-(tetrahydro-2H-pyran-4-yl)ethanol to obtain hydrochloride salt of compound 12, a white solid, with a one-step yield of 40% in step 6).

¹H NMR (600 MHz, Methanol-d₄) δ 7.76 (s, 1H), 7.52-7.45 (m, 1H), 7.35-7.28 (m, 1H), 6.93 (t, J=8.4 Hz, 1H), 6.89-6.84 (m, 1H), 6.67 (dd, J=8.4, 2.4 Hz, 1H), 6.51 (dd, J=11.4, 2.4 Hz, 1H), 6.39 (d, J=2.4 Hz, 1H), 4.28-4.20 (m, 1H), 4.10 (s, 2H), 3.97 (dd, J=11.4, 4.2 Hz, 2H), 3.46-3.34 (m, 2H), 2.70 (s, 3H), 1.88-1.77 (m, 2H), 1.65-1.55 (m, 1H), 1.54-1.36 (m, 2H), 1.26 (d, J=6.0 Hz, 3H).

### Preparation Example 13: The Preparation Example is Used to Describe the Synthesis Process of Hydrochloride Salt of Compound 13

The preparation method of this preparation example is similar to that of preparation example 1, with the following differences: Pyridine-3-sulfonyl chloride in step 3) was replaced with an equimolar amount of 3-cyano-benzenesulfonyl chloride, a yellow solid, with a yield of 71%.

Tert-butyl((5-bromo-1-(pyridin-3-ylsulfonyl)-1H-pyrrol-3-yl)methyl)(methyl)carbamate in step 5) was replaced with an equimolar amount of tert-butyl((5-bromo-1-(3-cyano-benzenesulfonyl)-1H-pyrrol-3-yl)methyl)(methyl)carbamate, a yellow solid, with a yield of 60%.

(2S)-1,4-dioxane-2-methanol in step 6) was replaced with an equimolar amount of 1-(tetrahydro-2H-pyran-4-yl)ethanol to obtain hydrochloride salt of compound 13, a white solid, with a one-step yield of 36% in step 6).

¹H NMR (600 MHz, Methanol-d₄) δ 8.84 (s, 1H), 8.59 (s, 1H), 7.94 (d, J=6.6 Hz, 1H), 7.79 (s, 1H), 7.60 (s, 1H), 6.97 (d, J=8.4 Hz, 1H), 6.73 (d, J=7.2 Hz, 1H), 6.62 (d, J=11.4 Hz, 1H), 6.39 (s, 1H), 4.33-4.24 (m, 1H), 4.10 (s, 2H), 2.71 (s, 3H), 1.99-1.90 (m, 1H), 1.85-1.76 (m, 3H), 1.73-1.68 (m, 1H), 1.66-1.56 (m, 1H), 1.38-1.05 (m, 8H).

### Preparation Example 14: The Preparation Example is Used to Describe the Synthesis Process of Hydrochloride Salt of Compound 14

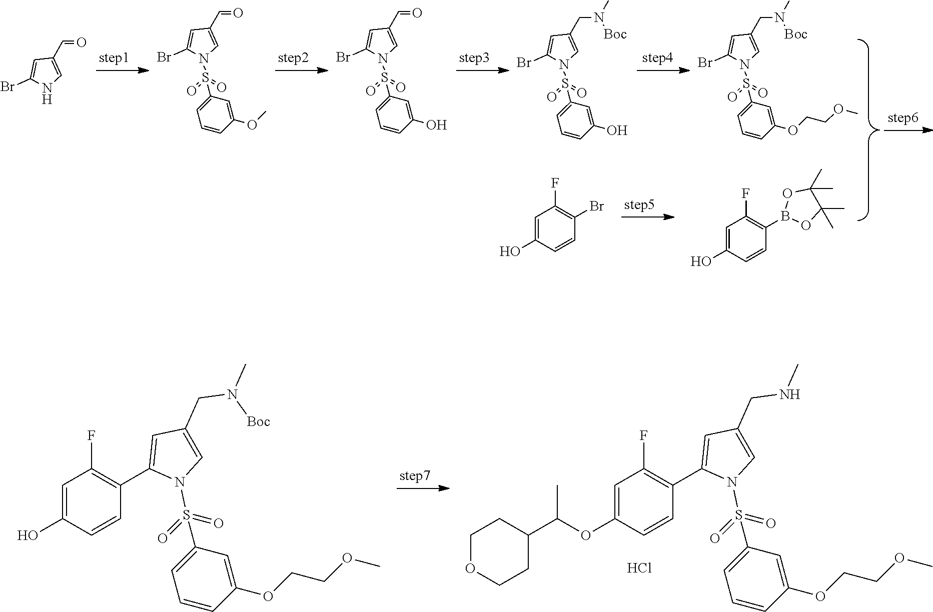

### Step 1: preparation of 5-bromo-1-((3-methoxyphenyl)sulfonyl)-1H-pyrrolo-3-formaldehyde

Pyrrolo-3-formaldehyde (11.5 mmol) dissolved in 25 mL of ultra-dry tetrahydrofuran was added to a one-neck round-bottom flask and cooled to 0° C., NaH (1.61 g, 60% in kerosene, 40.0 mmol) was added slowly and then 3-methoxybenzenesulfonyl chloride (17.2 mmol) was added dropwise. After the addition, the system was further stirred at 0° C. for 30 min and then stirred at room temperature for 30 min. After it was detected by TLC that the raw materials were fully reacted, the system was poured into ice water, extracted with ethyl acetate (200 mL×2) and washed with saturated brine (150×2), and the organic phases were merged, dried with anhydrous sodium sulfate, concentrated under reduced pressure and then purified by column chromatography to obtain a light yellow solid, with a yield of 90.0%.

### Step 2: preparation of 5-bromo-1-((3-hydroxyphenyl)sulfonyl)-1H-pyrrolo-3-formaldehyde

5-bromo-1-((3-methoxyphenyl)sulfonyl)-1H-pyrrolo-3-formaldehyde (10.3 mmol) and ultra-dry dichloromethane (CH₂Cl₂, 50 mL) were added to a three-neck round-bottom flask and cooled in a cryogenic magnetic stirrer to −78° C., and BBr₃ (51.7 mmol) was added dropwise to the reaction system. After the addition, the system was transferred to an ice bath and stirred for 30 min. After it was detected by TLC that the raw materials were fully reacted, the system was added with ice water, extracted with EA (200 mL×2) and washed with saturated brine (150 mL×2), and the organic phases were merged, dried with anhydrous sodium sulfate, concentrated under reduced pressure, stirred, purified by column chromatography and concentrated under reduced pressure to obtain a yellow solid, with a yield of 58.0%.

### Step 3: preparation of tert-butyl((5-bromo-1-((3-hydroxyphenyl)sulfonyl)-1H-pyrrol-3-yl)methyl)(methyl)carbamate

Methylamine hydrochloride (15.2 mmol) and methanol (CH₃OH, 50 mL) were added to a one-neck round-bottom flask and cooled in a cryogenic magnetic stirrer to −40° C., then 5-bromo-1-((3-hydroxyphenyl)sulfonyl)-1H-pyrrolo-3-formaldehyde (15.1 mmol) and cyanosodium borohydride (52.5 mmol) were added, and the temperature was naturally raised to room temperature. After it was detected by TLC that the raw materials were fully reacted, the reaction system was transferred with ethyl acetate (300 mL) to a separatory funnel in batches, and washed with saturated brine (150 mL×3), dried with anhydrous sodium sulfate, and concentrated under reduced pressure to obtain a brownish yellow oily substance, the above product was dissolved in acetonitrile (30 mL), bi-tert-butyl dicarbonate (17.3 mmol) and sodium bicarbonate (42.8 mmol) were added, and the solution was stirred in an oil bath at 50° C. overnight. After it was detected by TLC that the raw materials were fully reacted, the reaction system was transferred with ethyl acetate (300 mL) to a separatory funnel in batches and washed with saturated brine (150 mL×3), and the organic phase was dried with anhydrous sodium sulfate, concentrated under reduced pressure, stirred, purified by column chromatography, and concentrated under reduced pressure to obtain a brown oily liquid, with a yield of 44.0%.

### Step 4: preparation of tert-butyl((5-bromo-1-((3-(2-methoxyethoxy)phenyl) sulfonyl)-1H-pyrrol-3-yl)methyl)(methyl)carbamate

The stirrer was set to preheat at 80° C. Tert-butyl((5-bromo-1-((3-hydroxyphenyl)sulfonyl)-1H-pyrrol-3-yl)methyl)(methyl)carbamate (0.89 mmol), triphenylphosphine (1.79 mmol), ethylene glycol methyl ether (1.79 mmol) and 10 mL of toluene were added to a one-neck round-bottom flask, heated with stirring to dissolve. When the temperature rose to 80° C., diisopropyl azodicarboxylate (1.79 mmol) was added dropwise and stirred at 80° C. for 10 min. After it was detected by TLC that the raw materials were fully reacted, the system was directly concentrated under reduced pressure, 2 mL of dichloromethane was sampled by the wet method and the system was purified by column chromatography to obtain a white solid, with a yield of 70.0%.

### Step 5: preparation of 3-fluoro-4-(4,4,5,5-tetramethyl-1,3,2-dioxaborolan-2-yl) phenol

4-bromo-3-fluoro-phenol (262 mmol), bis(pinacolato)diboron (314 mmol), tetrakis(triphenylphosphine)palladium (13.1 mmol) and potassium acetate (524 mmol) were added to a reaction tube. 400 mL of 1,4-dioxane was added after nitrogen was pumped and charged for 3 times, and was stirred at 130° C. for 12 h. After it was detected by TLC that the raw materials were fully reacted, the system was added with ice water and extracted, and the organic phase was dried with anhydrous sodium sulfate, concentrated under reduced pressure and then purified by column chromatography to obtain a white solid, with a yield of 51.5%.

### Step 6: preparation of tert-butyl((1-((3-(2-methoxyethoxy)phenyl)sulfonyl)-5-(2-fluoro-4-hydroxyphenyl)-1H-pyrrol-3-yl)methyl)(methyl)carbamate

Tert-butyl((5-bromo-1-((3-(2-methoxyethoxy)phenyl)sulfonyl)-1H-pyrrol-3-yl)methyl)(methyl)carbamate (2.0 mmol), 3-fluoro-4-(4,4,5,5-tetramethyl-1,3,2-dioxaborolan-2-yl)phenol (3.0 mmol), tetrakis(triphenylphosphine)palladium (0.1 mmol) and sodium bicarbonate (6.10 mmol) were added to a reaction tube, nitrogen was pumped and charged for 3 times, DME (20 mL) and H₂O (5 mL) were added under nitrogen protection, and reaction was performed at 100° C. for 2 h. After it was detected by TLC that the raw materials were fully reacted, the reaction system was transferred with ethyl acetate (150 mL) to a separatory funnel, washed with saturated brine (100 mL×3), dried with anhydrous sodium sulfate and concentrated under reduced pressure, and the residue was purified by column chromatography to obtain a light yellow solid, with a yield of 69.3%.

### Step 7: 1-(5-(2-fluoro-4-(1-(tetrahydro-2H-pyran-4-yl)ethoxy)phenyl)-1-((3-(2-methoxyethoxy)phenyl)sulfonyl)-1H-pyrrol-3-yl)-N-methylmethylamine-hydrochloride

The stirrer was set to preheat at 90° C. Tert-butyl((1-((3-(2-methoxyethoxy)phenyl)sulfonyl)-5-(2-fluoro-4-hydroxyphenyl)-1H-pyrrol-3-yl)methyl)(methyl)carbamate (0.64 mmol), triphenylphosphine (1.28 mmol), 1-(tetrahydro-2H-pyran-4-yl)ethanol (1.12 mmol), and 10 mL of toluene were added to a one-neck round-bottom flask, heated, stirred and dissolved. When the temperature rose to 80° C., diisopropyl azodicarboxylate (1.28 mmol) was added dropwise and stirred at 80° C. for 5 min. After it was detected by TLC that the raw materials were fully reacted, the system was directly concentrated under reduced pressure to obtain a crude produce, 2 ml of ethyl acetate solution of hydrochloric acid and 2 mL of ethyl acetate were added and they were stirred at room temperature for 12 h. After it was detected by TLC that the raw materials were fully reacted, the system was filtered under vacuum to obtain a yellow oily substance, with a yield of 29%.

¹H NMR (600 MHz, Methanol-d₄) δ 7.69 (s, 1H), 7.38 (s, 1H), 7.25-7.18 (m, 1H), 7.11-7.05 (m, 1H), 6.92 (d, J=24.0 Hz, 2H), 6.75-6.69 (m, 1H), 6.66 (d, J=11.4 Hz, 1H), 6.32 (s, 1H), 4.34-4.26 (m, 1H), 4.08-3.96 (m, 7H), 3.72 (s, 2H), 3.50-3.36 (m, 2H), 2.66 (s, 3H), 1.88-1.81 (m, 2H), 1.69-1.60 (m, 1H), 1.57-1.43 (m, 2H), 1.30 (s, 5H).

### Preparation Example 15: The Preparation Example is Used to Describe the Synthesis Process of Hydrochloride Salt of Compound 15

The preparation method of this preparation example is similar to that of preparation example 1, with the following differences:

Pyridine-3-sulfonyl chloride in step 3) was replaced with an equimolar amount of 3-methoxybenzenesulfonyl chloride, a yellow solid, with a yield of 69%.

Tert-butyl((5-bromo-1-(pyridin-3-ylsulfonyl)-1H-pyrrol-3-yl)methyl)(methyl)carbamate in step 5) was replaced with an equimolar amount of tert-butyl((5-bromo-1-(3-methoxybenzenesulfonyl)-1H-pyrrol-3-yl)methyl)(methyl)carbamate, a yellow solid, with a yield of 55%.

(2S)-1,4-dioxane-2-methanol in step 6) was replaced with an equimolar amount of 1-(tetrahydro-2H-pyran-4-yl)ethanol to obtain hydrochloride salt of compound 15, a grey solid, with a one-step yield of 45% in step 6).

¹H NMR (600 MHz, Methanol-d₄) δ 7.72 (s, 1H), 7.37 (t, J=8.4 Hz, 1H), 7.17 (d, J=8.4 Hz, 1H), 7.07 (d, J=7.8 Hz, 1H), 6.93 (t, J=8.4 Hz, 1H), 6.84 (s, 1H), 6.69 (d, J=9.0 Hz, 1H), 6.62 (d, J=11.4 Hz, 1H), 6.33 (s, 1H), 4.31-4.23 (m, 1H), 4.06 (s, 2H), 3.98 (dd, J=11.4, 4.2 Hz, 2H), 3.73 (s, 3H), 3.47-3.38 (m, 2H), 2.68 (s, 3H), 1.90-1.78 (m, 2H), 1.67-1.59 (m, 1H), 1.55-1.39 (m, 2H), 1.28 (d, J=6.0 Hz, 3H).

### Preparation Example 16: The Preparation Example is Used to Describe the Synthesis Process of Hydrochloride Salt of Compound 16

The preparation method of this preparation example is similar to that of preparation example 1, with the following differences:

Pyridine-3-sulfonyl chloride in step 3) was replaced with an equimolar amount of 2,6-difluorobenzenesulfonyl chloride, a yellow solid, with a yield of 67%.

Tert-butyl((5-bromo-1-(pyridin-3-ylsulfonyl)-1H-pyrrol-3-yl)methyl)(methyl)carbamate in step 5) was replaced with an equimolar amount of tert-butyl((5-bromo-1-(2,6-difluorophenylsulfonyl)-1H-pyrrol-3-yl)methyl)(methyl)carbamate, a yellow solid, with a yield of 63%.

(2S)-1,4-dioxane-2-methanol in step 6) was replaced with an equimolar amount of 1-(tetrahydro-2H-pyran-4-yl)ethanol to obtain hydrochloride salt of compound 16, a white solid, with a one-step yield of 41% in step 6).

¹H NMR (600 MHz, Methanol-d₄) δ 7.71-7.57 (m, 2H), 6.95 (t, J=9.0 Hz, 2H), 6.82 (t, J=8.4 Hz, 1H), 6.55 (d, J=9.0 Hz, 1H), 6.44 (d, J=11.4 Hz, 1H), 6.31 (s, 1H), 4.19-4.14 (m, 1H), 4.03 (s, 2H), 3.90 (dd, J=11.4, 4.2 Hz, 2H), 3.34 (dt, J=15.6, 7.8 Hz, 2H), 2.63 (s, 3H), 1.81-1.70 (m, 2H), 1.57-1.50 (m, 1H), 1.46-1.30 (m, 2H), 1.18 (d, J=6.0 Hz, 3H).

### Preparation Example 17: The Preparation Example is Used to Describe the Synthesis Process of Hydrochloride Salt of Compound 17

The preparation method of this preparation example is similar to that of preparation example 1, with the following differences: Pyridine-3-sulfonyl chloride in step 3) was replaced with an equimolar amount of 2, 4-difluorobenzenesulfonyl chloride, a yellow solid, with a yield of 68%.

Tert-butyl((5-bromo-1-(pyridin-3-ylsulfonyl)-1H-pyrrol-3-yl)methyl)(methyl)carbamate in step 5) was replaced with an equimolar amount of tert-butyl((5-bromo-1-(2,4-difluorobenzenesulfonyl)-1H-pyrrol-3-yl)methyl)(methyl)carbamate, a yellow solid, with a yield of 65%.

(2S)-1,4-dioxane-2-methanol in step 6) was replaced with an equimolar amount of 1-(tetrahydro-2H-pyran-4-yl)ethanol to obtain hydrochloride salt of compound 17, a white solid, with a one-step yield of 42% in step 6).

¹H NMR (600 MHz, Methanol-d₄) δ 7.92 (s, 1H), 7.53-7.45 (m, 1H), 7.42-7.34 (m, 1H), 7.16-7.09 (m, 2H), 6.86-6.81 (m, 1H), 6.73-6.67 (m, 1H), 6.57-6.53 (m, 1H), 4.47-4.36 (m, 1H), 4.27 (s, 2H), 4.15 (dd, J=11.4, 4.2 Hz, 2H), 3.65-3.54 (m, 2H), 2.87 (s, 3H), 2.05-1.94 (m, 2H), 1.82-1.76 (m, 1H), 1.72-1.55 (m, 2H), 1.44 (d, J=6.0 Hz, 3H).

### Preparation Example 18: The Preparation Example is Used to Describe the Synthesis Process of Hydrochloride Salt of Compound 18

The preparation method of this preparation example is similar to that of preparation example 1, with the following differences:

Pyridine-3-sulfonyl chloride in step 3) was replaced with an equimolar amount of 4-cyano-benzenesulfonyl chloride, a yellow solid, with a yield of 66%.

Tert-butyl((5-bromo-1-(pyridin-3-ylsulfonyl)-1H-pyrrol-3-yl)methyl)(methyl)carbamate in step 5) was replaced with an equimolar amount of tert-butyl((5-bromo-1-(4-cyano-benzenesulfonyl)-1H-pyrrol-3-yl)methyl)(methyl)carbamate, a yellow solid, with a yield of 63%.

(2S)-1,4-dioxane-2-methanol in step 6) was replaced with an equimolar amount of 1-(tetrahydro-2H-pyran-4-yl)ethanol to obtain hydrochloride salt of compound 18, a white solid, with a one-step yield of 43% in step 6).

¹H NMR (600 MHz, Methanol-d₄) δ 8.02 (d, J=8.4 Hz, 2H), 7.95-7.90 (m, 1H), 7.79 (d, J=8.4 Hz, 2H), 7.12 (t, J=8.4 Hz, 1H), 6.89 (dd, J=8.4, 2.4 Hz, 1H), 6.81 (dd, J=12.0, 2.4 Hz, 1H), 6.55-6.51 (m, 1H), 4.50-4.44 (m, 1H), 4.24 (s, 2H), 4.15 (dd, J=11.4, 4.2 Hz, 2H), 3.65-3.56 (m, 2H), 2.86 (s, 3H), 2.08-1.96 (m, 2H), 1.82-1.77 (m, 1H), 1.72-1.57 (m, 2H), 1.46 (d, J=6.0 Hz, 3H).

### Preparation Example 19: The Preparation Example is Used to Describe the Synthesis Process of Hydrochloride Salt of Compound 19

The preparation method of this preparation example is similar to that of preparation example 1, with the following differences:

Pyridine-3-sulfonyl chloride in step 3) was replaced with an equimolar amount of 3-fluorobenzenesulfonyl chloride, a yellow solid, with a yield of 68%.

Tert-butyl((5-bromo-1-(pyridin-3-ylsulfonyl)-1H-pyrrol-3-yl)methyl)(methyl)carbamate in step 5) was replaced with an equimolar amount of tert-butyl((5-bromo-1-(3-fluorobenzenesulfonyl)-1H-pyrrol-3-yl)methyl)(methyl)carbamate, a yellow solid, with a yield of 61%.

(2S)-1,4-dioxane-2-methanol in step 6) was replaced with an equimolar amount of 1-(tetrahydro-2H-pyran-4-yl)ethanol to obtain hydrochloride salt of compound 19, a white solid, with a one-step yield of 48% in step 6).

¹H NMR (600 MHz, Methanol-d₄) δ 7.75 (d, J=1.8 Hz, 1H), 7.53 (td, J=8.14, 5.4 Hz, 1H), 7.44 (td, J=8.4, 2.4 Hz, 1H), 7.34 (d, J=7.8 Hz, 1H), 7.12 (dt, J=8.4, 2.4 Hz, 1H), 6.97 (t, J=8.4 Hz, 1H), 6.73 (dd, J=8.4, 2.4 Hz, 1H), 6.64 (dd, J=11.4, 2.4 Hz, 1H), 6.38 (d, J=2.4 Hz, 1H), 4.33-4.26 (m, 1H), 4.09 (s, 2H), 3.99 (dd, J=11.4, 4.2 Hz, 2H), 3.48-3.37 (m, 2H), 2.70 (s, 3H), 1.91-1.81 (m, 2H), 1.68-1.60 (m, 1H), 1.56-1.42 (m, 2H), 1.30 (d, J=6.0 Hz, 3H).

### Preparation Example 20: The Preparation Example is Used to Describe the Synthesis Process of Hydrochloride Salt of Compound 20

The preparation method of this preparation example is similar to that of preparation example 1, with the following differences:

(2S)-1,4-dioxane-2-methanol in step 6) was replaced with an equimolar amount of 1-cyclohexylethanol to obtain hydrochloride salt of compound 20, a white solid, with a one-step yield of 49% in step 6).

¹H NMR (600 MHz, Methanol-d₄) δ 8.84 (s, 1H), 8.59 (s, 1H), 7.94 (d, J=6.6 Hz, 1H), 7.79 (s, 1H), 7.60 (s, 1H), 6.97 (d, J=8.4 Hz, 1H), 6.73 (d, J=7.2 Hz, 1H), 6.62 (d, J=11.4 Hz, 1H), 6.39 (s, 1H), 4.33-4.24 (m, 1H), 4.10 (s, 2H), 2.71 (s, 3H), 1.99-1.90 (m, 1H), 1.85-1.76 (m, 3H), 1.73-1.68 (m, 1H), 1.66-1.56 (m, 1H), 1.38-1.05 (m, 8H).

### Preparation Example 21: The Preparation Example is Used to Describe the Synthesis Process of Hydrochloride Salt of Compound 21

The preparation method of this preparation example is similar to that of preparation example 1, with the following differences:

(2S)-1,4-dioxane-2-methanol in step 6) was replaced with an equimolar amount of 4-hydroxymethylpiperidine to obtain hydrochloride salt of compound 21, a yellow solid, with a one-step yield of 33% in step 6).

¹H NMR (600 MHz, Methanol-d₄) δ 8.79 (d, J=4.8 Hz, 1H), 8.50 (s, 1H), 7.96 (d, J=8.4 Hz, 1H), 7.75 (s, 1H), 7.63-7.54 (m, 2H), 6.97 (t, J=8.4 Hz, 1H), 6.74 (d, J=8.4 Hz, 1H), 6.66 (d, J=11.4 Hz, 1H), 6.36 (s, 1H), 4.05 (s, 2H), 3.93 (d, J=6.0 Hz, 2H), 3.46-3.38 (m, 2H), 3.09-2.99 (m, 2H), 2.66 (s, 3H), 2.20-2.12 (m, 1H), 2.09-2.02 (m, 2H), 1.70-1.52 (m, 2H).

### Preparation Example 22: The Preparation Example is Used to Describe the Synthesis Process of Hydrochloride Salt of Compound 22

The preparation method of this preparation example is similar to that of preparation example 1, with the following differences:

(2S)-1,4-dioxane-2-methanol in step 6) was replaced with an equimolar amount of 1-methyl-4-piperidinemethanol to obtain hydrochloride salt of compound 22, a grey solid, with a one-step yield of 40% in step 6).

¹H NMR (600 MHz, Methanol-d₄) δ 8.83 (d, J=5.4 Hz, 1H), 8.54 (s, 1H), 7.98 (d, J=8.4 Hz, 1H), 7.80 (s, 1H), 7.62 (s, 1H), 7.01 (d, J=8.4 Hz, 1H), 6.79 (d, J=8.4 Hz, 1H), 6.71 (d, J=11.2 Hz, 1H), 6.41 (d, J=2.4 Hz, 1H), 3.99 (d, J=5.4 Hz, 2H), 3.63-3.56 (m, 2H), 3.12-3.04 (m, 2H), 2.90 (s, 2H), 2.71 (s, 3H), 2.20-2.12 (m, 2H), 2.01 (s, 3H), 1.77-1.66 (m, 2H), 1.63-1.57 (m, 1H).

### Preparation Example 23

The preparation example is used to describe the synthesis process of hydrochloride salt of compound 23:

The preparation method of this preparation example is similar to that of preparation example 1, with the following differences:

(2S)-1,4-dioxane-2-methanol in step 6) was replaced with an equimolar amount of 1-(4-(hydroxymethyl)piperidin-1-yl)ethanone to obtain hydrochloride salt of compound 23, a white solid, with a one-step yield of 20% in step 6).

¹H NMR (400 MHz, Methanol-d₄) δ 8.81 (dd, J=4.8, 1.8 Hz, 1H), 8.54 (d, J=2.4 Hz, 1H), 8.00-7.94 (m, 1H), 7.77 (d, J=1.8 Hz, 1H), 7.64-7.57 (m, 1H), 6.97 (t, J=8.4 Hz, 1H), 6.74 (dd, J=8.4, 2.4 Hz, 1H), 6.65 (dd, J=11.4, 2.4 Hz, 1H), 6.37 (d, J=1.8 Hz, 1H), 4.55 (d, J=13.3 Hz, 1H), 4.06 (s, 2H), 3.99 (d, J=14.0 Hz, 1H), 3.90 (d, J=6.2 Hz, 2H), 3.19 (t, J=13.3 Hz, 1H), 2.79-2.62 (m, 4H), 2.14 (s, 3H), 2.13-2.05 (s, 1H), 2.02-1.84 (m, 2H), 1.47-1.32 (m, 2H).

### Preparation Example 24: The Preparation Example is Used to Describe the Synthesis Process of Hydrochloride Salt of Compound 24

The preparation method of this preparation example is similar to that of preparation example 1, with the following differences:

Pyridine-3-sulfonyl chloride in step 3) was replaced with an equimolar amount of 3-fluorobenzenesulfonyl chloride, a yellow solid, with a yield of 71%.

Tert-butyl((5-bromo-1-(pyridin-3-ylsulfonyl)-1H-pyrrol-3-yl)methyl)(methyl)carbamate in step 5) was replaced with an equimolar amount of tert-butyl((5-bromo-1-(3-fluorobenzenesulfonyl)-1H-pyrrol-3-yl)methyl)(methyl)carbamate, a yellow solid, with a yield of 68%.

(2S)-1,4-dioxane-2-methanol in step 6) was replaced with an equimolar amount of 4-hydroxymethyltetrahydropyran to obtain hydrochloride salt of compound 24, a white solid, with a one-step yield of 45% in step 6).

¹H NMR (600 MHz, Methanol-d₄) δ 7.74 (d, J=1.8 Hz, 1H), 7.68 (dd, J=7.8, 1.8 Hz, 1H), 7.53-7.41 (m, 2H), 7.30 (d, J=2.4 Hz, 1H), 6.97 (t, J=8.4 Hz, 1H), 6.76 (dd, J=8.4, 2.4 Hz, 1H), 6.66 (dd, J=11.4, 2.4 Hz, 1H), 6.36 (d, J=1.8 Hz, 1H), 4.08 (s, 2H), 3.99 (dd, J=11.4, 4.2 Hz, 2H), 3.90 (d, J=6.6 Hz, 2H), 3.48 (td, J=12.0, 2.4 Hz, 2H), 2.70 (s, 3H), 2.15-2.07 (m, 1H), 1.84-1.72 (m, 2H), 1.54-1.41 (m, 2H).

### Preparation Example 25: The Preparation Example is Used to Describe the Synthesis Process of Hydrochloride Salt of Compound 25

The preparation method of this preparation example is similar to that of preparation example 1, with the following differences:

Pyridine-3-sulfonyl chloride in step 3) was replaced with an equimolar amount of 3-chlorobenzenesulfonyl chloride, a yellow solid, with a yield of 72%.

Tert-butyl((5-bromo-1-(pyridin-3-ylsulfonyl)-1H-pyrrol-3-yl)methyl)(methyl)carbamate in step 5) was replaced with an equimolar amount of tert-butyl((5-bromo-1-(3-chlorobenzenesulfonyl)-1H-pyrrol-3-yl)methyl)(methyl)carbamate, a yellow solid, with a yield of 63%.

(2S)-1,4-dioxane-2-methanol in step 6) was replaced with an equimolar amount of 4-hydroxymethyltetrahydropyran to obtain hydrochloride salt of compound 25, a white solid, with a one-step yield of 42% in step 6).

¹H NMR (600 MHz, Methanol-d₄) δ 7.74 (d, J=1.8 Hz, 1H), 7.56-7.39 (m, 2H), 7.32 (d, J=7.8 Hz, 1H), 7.16-7.09 (m, 1H), 7.00-6.93 (m, 1H), 6.74 (dd, J=8.4, 2.4 Hz, 1H), 6.66 (dd, J=11.4, 2.4 Hz, 1H), 6.36 (d, J=1.8 Hz, 1H), 4.08 (s, 2H), 3.99 (dd, J=11.4, 4.2 Hz, 2H), 3.89 (d, J=6.6 Hz, 2H), 3.48 (td, J=12.0, 2.4 Hz, 2H), 2.70 (s, 3H), 2.15-2.03 (m, 1H), 1.82-1.71 (m, 2H), 1.53-1.41 (m, 2H).

### Preparation Example 26: The Preparation Example is Used to Describe the Synthesis Process of Hydrochloride Salt of Compound 26

The preparation method of this preparation example is similar to that of preparation example 1, with the following differences:

Pyridine-3-sulfonyl chloride in step 3) was replaced with an equimolar amount of 3-fluoro-4-methylbenzenesulfonyl chloride, a yellow solid, with a yield of 72%.

Tert-butyl((5-bromo-1-(pyridin-3-ylsulfonyl)-1H-pyrrol-3-yl)methyl)(methyl)carbamate in step 5) was replaced with an equimolar amount of tert-butyl((5-bromo-1-(3-fluoro-4-methylbenzenesulfonyl)-1H-pyrrol-3-yl)methyl)(methyl)carbamate, a yellow solid, with a yield of 65%.

(2S)-1,4-dioxane-2-methanol in step 6) was replaced with an equimolar amount of 4-hydroxymethyltetrahydropyran to obtain hydrochloride salt of compound 26, a white solid, with a one-step yield of 44% in step 6).

¹H NMR (600 MHz, Methanol-d₄) δ 7.71 (d, J=1.8 Hz, 1H), 7.37 (t, J=7.8 Hz, 1H), 7.20 (dd, J=8.4, 1.8 Hz, 1H), 7.06 (dd, J=8.4, 1.8 Hz, 1H), 6.98 (t, J=8.4 Hz, 1H), 6.74 (dd, J=8.4, 2.4 Hz, 1H), 6.67 (dd, J=11.4, 2.4 Hz, 1H), 6.34 (d, J=1.8 Hz, 1H), 4.07 (s, 2H), 4.02-3.96 (m, 2H), 3.89 (d, J=6.6 Hz, 2H), 3.48 (td, J=12.0, 2.4 Hz, 2H), 2.69 (s, 3H), 2.31 (s, 3H), 2.15-2.06 (m, 1H), 1.83-1.74 (m, 2H), 1.54-1.41 (m, 2H).

### Preparation Example 27: The Preparation Example is Used to Describe the Synthesis Process of Hydrochloride Salt of Compound 27

The preparation method of this preparation example is similar to that of preparation example 1, with the following differences:

Pyridine-3-sulfonyl chloride in step 3) was replaced with an equimolar amount of 3,5-difluorobenzenesulfonyl chloride, a yellow solid, with a yield of 71%.

Tert-butyl((5-bromo-1-(pyridin-3-ylsulfonyl)-1H-pyrrol-3-yl)methyl)(methyl)carbamate in step 5) was replaced with an equimolar amount of tert-butyl((5-bromo-1-(3,5-difluorobenzenesulfonyl)-1H-pyrrol-3-yl)methyl)(methyl)carbamate, a yellow solid, with a yield of 61%.

(2S)-1,4-dioxane-2-methanol in step 6) was replaced with an equimolar amount of 4-hydroxymethyltetrahydropyran to obtain hydrochloride salt of compound 27, a white solid, with a one-step yield of 49% in step 6).

¹H NMR (600 MHz, Methanol-d₄) δ 7.75 (d, J=1.8 Hz, 1H), 7.43-7.34 (m, 1H), 7.10-6.99 (m, 3H), 6.77 (dd, J=8.4, 2.4 Hz, 1H), 6.68 (dd, J=11.4, 2.4 Hz, 1H), 6.40 (d, J=2.4 Hz, 1H), 4.09 (s, 2H), 3.99 (dd, J=11.4, 4.2 Hz, 2H), 3.90 (d, J=6.6 Hz, 2H), 3.48 (td, J=12.0, 1.8 Hz, 2H), 2.71 (s, 3H), 2.15-2.05 (m, 1H), 1.83-1.74 (m, 2H), 1.53-1.42 (m, 2H).

### Preparation Example 28: The Preparation Example is Used to Describe the Synthesis Process of Hydrochloride Salt of Compound 28

The preparation method of this preparation example is similar to that of preparation example 1, with the following differences:

Pyridine-3-sulfonyl chloride in step 3) was replaced with an equimolar amount of 3-cyano-benzenesulfonyl chloride, a yellow solid, with a yield of 70%.

Tert-butyl((5-bromo-1-(pyridin-3-ylsulfonyl)-1H-pyrrol-3-yl)methyl)(methyl)carbamate in step 5) was replaced with an equimolar amount of tert-butyl((5-bromo-1-(3-cyano-benzenesulfonyl)-1H-pyrrol-3-yl)methyl)(methyl)carbamate, a yellow solid, with a yield of 63%.

(2S)-1,4-dioxane-2-methanol in step 6) was replaced with an equimolar amount of 4-hydroxymethyltetrahydropyran to obtain hydrochloride salt of compound 28, a white solid, with a one-step yield of 51% in step 6).

¹H NMR (600 MHz, Methanol-d₄) δ 8.02 (d, J=7.8 Hz, 1H), 7.81-7.74 (m, 2H), 7.69 (t, J=7.8 Hz, 1H), 7.59 (s, 1H), 6.98 (t, J=8.4 Hz, 1H), 6.77 (d, J=8.4 Hz, 1H), 6.66 (d, J=11.4 Hz, 1H), 6.36 (d, J=1.8 Hz, 1H), 4.08 (s, 2H), 3.99 (d, J=11.4 Hz, 2H), 3.92 (d, J=6.6 Hz, 2H), 3.48 (t, J=12.0 Hz, 2H), 2.70 (s, 3H), 2.15-2.05 (m, 1H), 1.82-1.74 (m, 2H), 1.54-1.44 (m, 2H).

### Preparation Example 29: The Preparation Example is Used to Describe the Synthesis Process of Hydrochloride Salt of Compound 29

The preparation method of this preparation example is similar to that of preparation example 1, with the following differences:

Pyridine-3-sulfonyl chloride in step 3) was replaced with an equimolar amount of 4-cyano-benzenesulfonyl chloride, a yellow solid, with a yield of 68%.

Tert-butyl((5-bromo-1-(pyridin-3-ylsulfonyl)-1H-pyrrol-3-yl)methyl)(methyl)carbamate in step 5) was replaced with an equimolar amount of tert-butyl((5-bromo-1-(4-cyano-benzenesulfonyl)-1H-pyrrol-3-yl)methyl)(methyl)carbamate, a yellow solid, with a yield of 59%.

(2S)-1,4-dioxane-2-methanol in step 6) was replaced with an equimolar amount of 4-hydroxymethyltetrahydropyran to obtain hydrochloride salt of compound 29, a white solid, with a one-step yield of 42% in step 6).

¹H NMR (600 MHz, Methanol-d₄) δ 7.91-7.84 (m, 2H), 7.76 (d, J=1.8 Hz, 1H), 7.65-7.60 (m, 2H), 6.96 (t, J=8.4 Hz, 1H), 6.74 (dd, J=8.4, 2.4 Hz, 1H), 6.67 (dd, J=11.4, 2.4 Hz, 1H), 6.37 (d, J=1.8 Hz, 1H), 4.08 (s, 2H), 3.99 (dd, J=11.4, 4.2 Hz, 2H), 3.89 (d, J=6.6 Hz, 2H), 3.48 (td, J=12.0, 2.4 Hz, 2H), 2.70 (s, 2H), 2.14-2.04 (m, 1H), 1.83-1.73 (m, 2H), 1.53-1.40 (m, 2H).

### Preparation Example 30: The Preparation Example is Used to Describe the Synthesis Process of Hydrochloride Salt of Compound 30

The preparation method of this preparation example is similar to that of preparation example 14, with the following differences:

Ethylene glycol methyl ether in step 4) was replaced with an equimolar amount of 2-fluoroethanol, a yellow solid, with a yield of 60%.

Tert-butyl((5-bromo-1-((3-(2-methoxyethoxy)phenyl)sulfonyl)-1H-pyrrol-3-yl)methyl)(methyl)carbamate in step 6) was replaced with an equimolar amount of tert-butyl((5-bromo-1-((3-(2-fluoroethoxy)phenyl)sulfonyl)-1H-pyrrol-3-yl)methyl)(methyl)carbamate, a yellow solid, with a yield of 60%.

1-(tetrahydro-2H-pyran-4-yl)ethanol in step 7) was replaced with an equimolar amount of 4-hydroxymethyltetrahydropyran to obtain hydrochloride salt of compound 30, a white solid, with a one-step yield of 43% in step 7).

¹H NMR (600 MHz, Methanol-d₄) δ 7.74 (d, J=1.8 Hz, 1H), 7.40 (t, J=8.4 Hz, 1H), 7.24 (dd, J=8.4, 2.6 Hz, 1H), 7.11 (d, J=8.4 Hz, 1H), 6.94 (t, J=8.4 Hz, 1H), 6.89 (t, J=2.4 Hz, 1H), 6.72 (dd, J=8.4, 2.4 Hz, 1H), 6.65 (dd, J=11.4, 2.4 Hz, 1H), 6.34 (d, J=1.8 Hz, 1H), 4.80-4.64 (m, 2H), 4.22-4.11 (m, 2H), 4.08 (s, 2H), 4.03-3.95 (m, 2H), 3.88 (d, J=6.0 Hz, 2H), 3.53-3.44 (m, 2H), 2.69 (s, 3H), 2.17-2.02 (m, 1H), 1.85-1.72 (m, 2H), 1.54-1.39 (m, 2H).

### Preparation Example 31: The Preparation Example is Used to Describe the Synthesis Process of Hydrochloride Salt of Compound 31

The preparation method of this preparation example is similar to that of preparation example 14, with the following differences:

Ethylene glycol methyl ether in step 4) was replaced with an equimolar amount of n-pentanol, a yellow solid, with a yield of 62%.

Tert-butyl((5-bromo-1-((3-(2-methoxyethoxy)phenyl)sulfonyl)-1H-pyrrol-3-yl)methyl)(methyl)carbamate in step 6) was replaced with an equimolar amount of tert-butyl((5-bromo-1-((3-(pentyloxy)phenyl)sulfonyl)-1H-pyrrol-3-yl)methyl)(methyl)carbamate, a yellow solid, with a yield of 62%.

1-(tetrahydro-2H-pyran-4-yl)ethanol in step 7) was replaced with an equimolar amount of 4-hydroxymethyltetrahydropyran to obtain hydrochloride salt of compound 31, a white solid, with a one-step yield of 20% in step 7).

¹H NMR (600 MHz, Methanol-d₄) δ 7.72 (d, J=1.8 Hz, 1H), 7.37 (t, J=8.4 Hz, 1H), 7.17 (dd, J=8.4, 2.4 Hz, 1H), 7.08 (d, J=7.8 Hz, 1H), 6.96 (t, J=8.4 Hz, 1H), 6.82 (t, J=2.4 Hz, 1H), 6.72 (dd, J=8.4, 2.4 Hz, 1H), 6.66 (dd, J=11.4, 2.4 Hz, 1H), 6.32 (d, J=2.4 Hz, 1H), 4.07 (s, 2H), 4.00 (dd, J=12.0, 4.2 Hz, 2H), 3.91-3.81 (m, 4H), 3.48 (t, J=12.0 Hz, 2H), 2.69 (s, 3H), 2.16-2.05 (m, 1H), 1.83-1.71 (m, 4H), 1.54-1.36 (m, 6H), 0.96 (t, J=7.0 Hz, 3H).

### Preparation Example 32: The Preparation Example is Used to Describe the Synthesis Process of Hydrochloride Salt of Compound 32

The preparation method of this preparation example is similar to that of preparation example 14, with the following differences:

Ethylene glycol methyl ether in step 4) was replaced with an equimolar amount of 3-methoxy-1-propanol, a yellow solid, with a yield of 64%.

Tert-butyl((5-bromo-1-((3-(2-methoxyethoxy)phenyl)sulfonyl)-1H-pyrrol-3-yl)methyl)(methyl)carbamate in step 6) was replaced with an equimolar amount of 1-(5-bromo-1-((3-(3-methoxypropoxy)phenyl)sulfonyl)-1H-pyrrol-3-yl)-N-methylmethylamine, a yellow solid, with a yield of 64%.

1-(tetrahydro-2H-pyran-4-yl)ethanol in step 7) was replaced with an equimolar amount of 4-hydroxymethyltetrahydropyran to obtain hydrochloride salt of compound 32, a white solid, with a one-step yield of 25% in step 7).

¹H NMR (600 MHz, Methanol-d₄) δ 7.71 (s, 1H), 7.38 (t, J=8.4 Hz, 1H), 7.19 (d, J=8.4 Hz, 1H), 7.09 (d, J=7.8 Hz, 1H), 6.96 (t, J=8.4 Hz, 1H), 6.87-6.81 (m, 1H), 6.73 (d, J=8.4 Hz, 1H), 6.68-6.61 (m, 1H), 6.32 (s, 1H), 4.07 (s, 2H), 4.02-3.94 (m, 4H), 3.89 (d, J=6.6 Hz, 2H), 3.55 (t, J=6.0 Hz, 2H), 3.49 (t, J=12.0 Hz, 2H), 3.35 (s, 3H), 2.69 (s, 3H) 2.15-2.06 (m, 1H), 2.04-1.96 (m, 2H), 1.82-1.74 (m, 2H), 1.53-1.43 (m, 2H).

### Preparation Example 33: The Preparation Example is Used to Describe the Synthesis Process of Hydrochloride Salt of Compound 33

The preparation method of this preparation example is similar to that of preparation example 14, with the following differences:

Ethylene glycol methyl ether in step 4) was replaced with an equimolar amount of cyclopropylmethanol, a yellow solid, with a yield of 61%.

Tert-butyl((5-bromo-1-((3-(2-methoxyethoxy)phenyl)sulfonyl)-1H-pyrrol-3-yl)methyl)(methyl)carbamate in step 6) was replaced with an equimolar amount of 1-(5-bromo-1-((3-(cyclobutylmethoxy)phenyl)sulfonyl)-1H-pyrrol-3-yl)-N-methylmethylamine, a yellow solid, with a yield of 62%.

1-(tetrahydro-2H-pyran-4-yl)ethanol in step 7) was replaced with an equimolar amount of 1-(tetrahydro-2H-pyran-4-yl)ethanol to obtain hydrochloride salt of compound 33, a white solid, with a one-step yield of 34% in step 7).

¹H NMR (400 MHz, Methanol-d₄) δ 7.75 (d, J=1.8 Hz, 1H), 7.39 (t, J=8.4 Hz, 1H), 7.21 (dd, J=8.4, 2.54z, 1H), 7.13-7.07 (m, 1H), 6.97 (t, J=8.4 Hz, 1H), 6.88 (t, J=2.4 Hz, 1H), 6.73 (dd, J=8.4, 2.4 Hz, 1H), 6.67 (dd, J=11.4, 2.4 Hz, 1H), 6.36 (d, J=2.4 Hz, 1H), 4.38-4.29 (m, 1H), 4.10 (s, 2H), 4.02 (dd, J=11.4, 4.2 Hz, 2H), 3.88 (d, J=6.6 Hz, 2H), 3.53-3.40 (m, 2H), 2.82-2.67 (m, 4H), 2.20-2.10 (m, 2H), 2.08-1.80 (m, 6H), 1.70-1.62 (m, 1H), 1.60-1.42 (m, 2H), 1.33 (d, J=6.0 Hz, 3H).

### Preparation Example 34: The Preparation Example is Used to Describe the Synthesis Process of Hydrochloride Salt of Compound 34

The preparation method of this preparation example is similar to that of preparation example 14, with the following differences:

Ethylene glycol methyl ether in step 4) was replaced with an equimolar amount of cyclopentylmethanol, a yellow solid, with a yield of 61%.

Tert-butyl((5-bromo-1-((3-(2-methoxyethoxy)phenyl)sulfonyl)-1H-pyrrol-3-yl)methyl)(methyl)carbamate in step 6) was replaced with an equimolar amount of 1-(5-bromo-1-((3-(cyclopentylmethoxy)phenyl)sulfonyl)-1H-pyrrol-3-yl)-N-methylmethylamine, a yellow solid, with a yield of 62%.

1-(tetrahydro-2H-pyran-4-yl)ethanol in step 7) was replaced with an equimolar amount of 4-hydroxymethyltetrahydropyran to obtain hydrochloride salt of compound 34, a white solid, with a one-step yield of 46% in step 7).

¹H NMR (400 MHz, Methanol-d₄) δ 7.72 (d, J=2.0 Hz, 1H), 7.37 (t, J=8.4 Hz, 1H), 7.17 (dd, J=8.4, 2.4 Hz, 1H), 7.09 (dd, J=7.6, 1.6 Hz, 1H), 6.96 (t, J=8.4 Hz, 1H), 6.81 (t, J=2.4 Hz, 1H), 6.72 (dd, J=8.4, 2.4 Hz, 1H), 6.66 (dd, J=11.4, 2.4 Hz, 1H), 6.33 (d, J=1.6 Hz, 1H), 4.07 (s, 2H), 3.99 (dd, J=11.4, 4.2 Hz, 2H), 3.88 (d, J=6.6 Hz, 2H), 3.75 (d, J=6.6 Hz, 2H), 3.48 (td, J=12.0, 1.8 Hz, 2H), 2.69 (s, 3H), 2.38-2.25 (m, 1H), 2.15-2.01 (m, 1H), 1.91-1.74 (m, 4H), 1.73-1.56 (m, 4H), 1.54-1.41 (m, 2H), 1.40-1.29 (m, 2H).

### Preparation Example 35: The Preparation Example is Used to Describe the Synthesis Process of Hydrochloride Salt of Compound 35

The preparation method of this preparation example is similar to that of preparation example 14, with the following differences:

Ethylene glycol methyl ether in step 4) was replaced with an equimolar amount of 2-fluoroethanol, a yellow solid, with a yield of 62%.

Tert-butyl((5-bromo-1-((3-(2-methoxyethoxy)phenyl)sulfonyl)-1H-pyrrol-3-yl)methyl)(methyl)carbamate in step 6) was replaced with an equimolar amount of 1-(5-bromo-1-((3-(2-fluoroethoxy)phenyl)sulfonyl)-1H-pyrrol-3-yl)-N-methylmethylamine, a yellow solid, with a yield of 61%.

1-(tetrahydro-2H-pyran-4-yl)ethanol in step 7) was replaced with an equimolar amount of cyclohexylmethanol to obtain hydrochloride salt of compound 35, a white solid, with a one-step yield of 45% in step 7).

¹H NMR (600 MHz, Methanol-d₄) δ 7.73 (d, J=1.8 Hz, 1H), 7.40 (t, J=8.4 Hz, 1H), 7.24 (dd, J=8.4, 2.4 Hz, 1H), 7.10 (d, J=7.8 Hz, 1H), 6.94 (t, J=8.4 Hz, 1H), 6.89 (t, J=2.4 Hz, 1H), 6.70 (dd, J=8.4, 2.4 Hz, 1H), 6.62 (dd, J=11.4, 2.4 Hz, 1H), 6.33 (d, J=1.8 Hz, 1H), 4.80-4.62 (m, 2H), 4.21-4.11 (m, 2H), 4.08 (s, 2H), 3.81 (d, J=6.6 Hz, 2H), 2.69 (s, 3H), 1.94-1.77 (m, 6H), 1.41-1.21 (m, 4H), 1.18-1.07 (m, 2H).

### Preparation Example 36: The Preparation Example is Used to Describe the Synthesis Process of Hydrochloride Salt of Compound 36

The preparation method of this preparation example is similar to that of preparation example 14, with the following differences:

Ethylene glycol methyl ether in step 4) was replaced with an equimolar amount of pentanol, a yellow solid, with a yield of 64%.

Tert-butyl((5-bromo-1-((3-(2-methoxyethoxy)phenyl)sulfonyl)-1H-pyrrol-3-yl)methyl)(methyl)carbamate in step 6) was replaced with an equimolar amount of 1-(5-bromo-1-((3-(pentyloxy)phenyl)sulfonyl)-1H-pyrrol-3-yl)-N-methylmethylamine, a yellow solid, with a yield of 60%.

1-(tetrahydro-2H-pyran-4-yl)ethanol in step 7) was replaced with an equimolar amount of cyclohexylmethanol to obtain hydrochloride salt of compound 36, a white solid, with a one-step yield of 19% in step 7).

¹H NMR (600 MHz, Methanol-d₄) δ 7.73 (d, J=1.8 Hz, 1H), 7.37 (t, J=7.8 Hz, 1H), 7.17 (dd, J=8.4, 2.4 Hz, 1H), 7.09 (d, J=7.8 Hz, 1H), 6.94 (t, J=8.4 Hz, 1H), 6.80 (t, J=2.4 Hz, 1H), 6.70 (dd, J=8.4, 2.4 Hz, 1H), 6.62 (dd, J=11.4, 2.4 Hz, 1H), 6.33 (d, J=1.8 Hz, 1H), 4.08 (s, 2H), 3.85 (t, J=6.6 Hz, 2H), 3.81 (d, J=6.0 Hz, 2H), 2.70 (s, 3H), 1.94-1.86 (m, 2H), 1.86-1.68 (m, 6H), 1.50-1.20 (m, 7H), 1.18-1.07 (m, 2H), 0.97 (t, J=7.0 Hz, 3H).

### Preparation Example 37: The Preparation Example is Used to Describe the Synthesis Process of Hydrochloride Salt of Compound 37

The preparation method of this preparation example is similar to that of preparation example 14, with the following differences:

Ethylene glycol methyl ether in step 4) was replaced with an equimolar amount of 3-methoxy-1-propanol, a yellow solid, with a yield of 61%.

Tert-butyl((5-bromo-1-((3-(2-methoxyethoxy)phenyl)sulfonyl)-1H-pyrrol-3-yl)methyl)(methyl)carbamate in step 6) was replaced with an equimolar amount of 1-(5-bromo-1-((3-(3-methoxypropoxy)phenyl)sulfonyl)-1H-pyrrol-3-yl)-N-methylmethylamine, a yellow solid, with a yield of 61%.

1-(tetrahydro-2H-pyran-4-yl)ethanol in step 7) was replaced with an equimolar amount of 1-(tetrahydro-2H-pyran-4-yl)ethanol to obtain hydrochloride salt of compound 37, a white solid, with a one-step yield of 44% in step 7).

¹H NMR (400 MHz, Methanol-d₄) δ 7.73 (d, J=1.8 Hz, 1H), 7.38 (t, J=8.4 Hz, 1H), 7.19 (dd, J=8.4, 2.4 Hz, 1H), 7.12-7.07 (m, 1H), 6.96 (t, J=8.4 Hz, 1H), 6.87 (t, J=2.4 Hz, 1H), 6.72 (dd, J=8.4, 2.4 Hz, 1H), 6.66 (dd, J=11.4, 2.4 Hz, 1H), 6.34 (d, J=1.8 Hz, 1H), 4.36-4.27 (m, 1H), 4.08 (s, 2H), 4.04-3.94 (m, 4H), 3.55 (t, J=6.0 Hz, 2H), 3.50-3.41 (m, 2H), 3.35 (s, 3H), 2.70 (s, 3H), 2.06-1.97 (m, 2H), 1.93-1.82 (m, 2H), 1.70-1.62 (m, 1H), 1.59-1.41 (m, 2H), 1.31 (d, J=6.0 Hz, 3H).

### Preparation Example 38: The Preparation Example is Used to Describe the Synthesis Process of Hydrochloride Salt of Compound 38

The preparation method of this preparation example is similar to that of preparation example 14, with the following differences:

Ethylene glycol methyl ether in step 4) was replaced with an equimolar amount of -methoxy-1-butanol, a yellow solid, with a yield of 58%.

Tert-butyl((5-bromo-1-((3-(2-methoxyethoxy)phenyl)sulfonyl)-1H-pyrrol-3-yl)methyl)(methyl)carbamate in step 6) was replaced with an equimolar amount of 1-(5-bromo-1-((3-(4-methoxybutoxy)phenyl)sulfonyl)-1H-pyrrol-3-yl)-N-methylmethylamine, a yellow solid, with a yield of 61%.

1-(tetrahydro-2H-pyran-4-yl)ethanol in step 7) was replaced with an equimolar amount of 1-(tetrahydro-2H-pyran-4-yl)ethanol to obtain hydrochloride salt of compound 38, a yellow solid, with a one-step yield of 20% in step 7).

¹H NMR (400 MHz, Methanol-d₄) δ 7.67 (d, J=1.8 Hz, 1H), 7.35 (t, J=8.4 Hz, 1H), 7.16 (dd, J=8.4, 2.4 Hz, 1H), 7.09-7.05 (m, 1H), 6.94 (t, J=8.4 Hz, 1H), 6.84 (t, J=2.4 Hz, 1H), 6.69 (dd, J=8.4, 2.4 Hz, 1H), 6.63 (dd, J=11.4, 2.4 Hz, 1H), 6.31 (d, J=1.8 Hz, 1H), 4.34-4.23 (m, 1H), 4.04-3.95 (m, 4H), 3.91 (t, J=6.0 Hz, 2H), 3.48-3.41 (m, 4H), 3.33 (s, 3H), 2.62 (s, 3H), 1.87-1.76 (m, 4H), 1.75-1.67 (m, 2H), 1.67-1.60 (m, 1H), 1.56-1.41 (m, 4H), 1.29 (d, J=6.0 Hz, 3H).

### Preparation Example 39: The Preparation Example is Used to Describe the Synthesis Process of Hydrochloride Salt of Compound 39

The preparation method of this preparation example is similar to that of preparation example 1, with the following differences:

Pyridine-3-sulfonyl chloride in step 3) was replaced with an equimolar amount of 3-trifluoromethylbenzenesulfonyl chloride, a yellow solid, with a yield of 68%.

Tert-butyl((5-bromo-1-(pyridin-3-ylsulfonyl)-1H-pyrrol-3-yl)methyl)(methyl)carbamate in step 5) was replaced with an equimolar amount of tert-butyl((5-bromo-1-(3-trifluoromethylbenzenesulfonyl)-1H-pyrrol-3-yl)methyl)(methyl)carbamate, a yellow solid, with a yield of 65%.

(2S)-1,4-dioxane-2-methanol in step 6) was replaced with an equimolar amount of 4-hydroxymethyltetrahydropyran to obtain hydrochloride salt of compound 39, a white solid, with a one-step yield of 54% in step 6).

¹H NMR (600 MHz, Methanol-d₄) δ 7.99 (d, J=7.8 Hz, 1H), 7.86-7.68 (m, 3H), 7.56 (s, 1H), 6.97 (t, J=8.4 Hz, 1H), 6.73 (dd, J=8.4, 2.4 Hz, 1H), 6.62 (dd, J=11.4, 2.4 Hz, 1H), 6.37 (d, J=1.8 Hz, 1H), 4.09 (s, 2H), 4.00 (dd, J=10.8, 4.2 Hz, 2H), 3.88 (d, J=6.6 Hz, 2H), 3.49 (td, J=12.0, 2.4 Hz, 2H), 2.70 (s, 3H), 2.15-2.05 (m, 1H), 1.83-1.73 (m, 2H), 1.53-1.42 (m, 2H).

### Preparation Example 40: The Preparation Example is Used to Describe the Synthesis Process of Hydrochloride Salt of Compound 40

The preparation method of this preparation example is similar to that of preparation example 1, with the following differences:

(2S)-1,4-dioxane-2-methanol in step 6) was replaced with an equimolar amount of methanol to obtain hydrochloride salt of compound 40, a white solid, with a one-step yield of 46% in step 6).

¹H NMR (600 MHz, Methanol-d₄) δ 9.01 (d, J=5.4 Hz, 1H), 8.81 (s, 1H), 8.35 (d, J=8.4 Hz, 1H), 7.96 (dd, J=8.4, 5.4 Hz, 1H), 7.89-7.82 (m, 1H), 7.04 (t, J=8.4 Hz, 1H), 6.78 (dd, J=8.4, 2.4 Hz, 1H), 6.71 (dd, J=11.4, 2.4 Hz, 1H), 6.47 (d, J=1.8 Hz, 1H), 4.12 (s, 2H), 3.87 (s, 3H), 2.72 (s, 3H).

### Preparation Example 41: The Preparation Example is Used to Describe the Synthesis Process of Hydrochloride Salt of Compound 41

The preparation method of this preparation example is similar to that of preparation example 1, with the following differences:

(2S)-1,4-dioxane-2-methanol in step 6) was replaced with an equimolar amount of ethanol to obtain hydrochloride salt of compound 41, a white solid, with a one-step yield of 53% in step 6).

¹H NMR (600 MHz, DMSO-d₆) δ 8.86 (s, 1H), 8.56 (s, 1H), 7.86 (d, J=7.8 Hz, 1H), 7.66-7.49 (m, 2H), 7.04-6.93 (m, 1H), 6.88-6.71 (m, 2H), 6.34 (s, 1H), 4.19-3.97 (m, 2H), 3.61 (s, 2H), 2.30 (s, 3H), 1.30-1.22 (m, 3H).

### Preparation Example 42: The Preparation Example is Used to Describe the Synthesis Process of Hydrochloride Salt of Compound 42

The preparation method of this preparation example is similar to that of preparation example 1, with the following differences:

(2S)-1,4-dioxane-2-methanol in step 6) was replaced with an equimolar amount of 2,2-difluoroethanol to obtain hydrochloride salt of compound 42, a white solid, with a one-step yield of 47% in step 6).

¹H NMR (600 MHz, DMSO-d₆) δ 8.86 (d, J=4.8 Hz, 1H), 8.57 (d, J=2.4 Hz, 1H), 7.87 (d, J=8.4 Hz, 1H), 7.61 (dd, J=8.4, 4.8 Hz, 1H), 7.48 (s, 1H), 7.04 (t, J=8.4 Hz, 1H), 6.97 (d, J=11.4 Hz, 1H), 6.86 (dd, J=8.4, 2.4 Hz, 1H), 6.43 (t, J=55.2 Hz, 1H), 6.32 (s, 1H), 4.46-4.35 (m, 2H), 3.51 (s, 2H), 2.24 (s, 3H).

### Preparation Example 43: The Preparation Example is Used to Describe the Synthesis Process of Hydrochloride Salt of Compound 43

The preparation method of this preparation example is similar to that of preparation example 1, with the following differences:

(2S)-1,4-dioxane-2-methanol in step 6) was replaced with an equimolar amount of 3-methoxypropanol to obtain hydrochloride salt of compound 43, a yellow solid, with a one-step yield of 51% in step 6).

¹H NMR (600 MHz, DMSO-d₆) δ 8.86 (d, J=4.8 Hz, 1H), 8.56 (s, 1H), 7.87 (d, J=8.4 Hz, 1H), 7.61 (t, J=6.6 Hz, 1H), 7.45 (s, 1H), 6.99 (t, J=8.4 Hz, 1H), 6.83 (d, J=11.4 Hz, 1H), 6.77 (d, J=8.4 Hz, 1H), 6.30 (s, 1H), 4.12-4.03 (m, 2H), 3.49 (d, J=6.0 Hz, 4H), 3.26 (s, 3H), 2.24 (s, 3H), 2.02-1.93 (m, 2H).

### Preparation Example 44: The Preparation Example is Used to Describe the Synthesis Process of Hydrochloride Salt of Compound 44

The preparation method of this preparation example is similar to that of preparation example 1, with the following differences:

(2S)-1,4-dioxane-2-methanol in step 6) was replaced with an equimolar amount of isopropanol to obtain hydrochloride salt of compound 44, a white solid, with a one-step yield of 50% in step 6).

¹H NMR (600 MHz, Methanol-d₄) δ 8.81 (d, J=4.8 Hz, 1H), 8.55 (s, 1H), 7.90 (d, J=8.4 Hz, 1H), 7.78 (s, 1H), 7.59-7.52 (m, 1H), 6.99 (t, J=8.4 Hz, 1H), 6.75 (d, J=8.4 Hz, 1H), 6.65 (d, J=11.4 Hz, 1H), 6.38 (s, 1H), 4.09 (s, 2H), 3.80 (d, J=6.6 Hz, 2H), 2.71 (s, 3H), 2.15-2.04 (m, 1H), 1.06 (dd, J=6.7, 2.4 Hz, 6H).

### Preparation Example 45: The Preparation Example is Used to Describe the Synthesis Process of Hydrochloride Salt of Compound 45

The preparation method of this preparation example is similar to that of preparation example 1, with the following differences:

(2S)-1,4-dioxane-2-methanol in step 6) was replaced with an equimolar amount of neopentanol to obtain hydrochloride salt of compound 45, a white solid, with a one-step yield of 38% in step 6).

¹H NMR (600 MHz, Methanol-d₄) δ 8.85 (d, J=4.8 Hz, 1H), 8.65-8.58 (m, 1H), 8.01 (d, J=8.4 Hz, 1H), 7.79 (s, 1H), 7.65 (dd, J=8.4, 4.8 Hz, 1H), 6.99 (t, J=8.4 Hz, 1H), 6.77 (d, J=8.4 Hz, 1H), 6.71-6.64 (m, 1H), 6.44-6.35 (m, 1H), 4.09 (s, 2H), 3.69 (s, 2H), 2.71 (s, 3H), 1.07 (s, 9H).

### Preparation Example 46: The Preparation Example is Used to Describe the Synthesis Process of Compound 46

The preparation method of this preparation example is similar to that of preparation example 1, with the following differences:

33% methylamine methanol solution in step 2) was replaced with an equimolar amount of ethylamine methanol, a brown oily liquid, with a yield of 70%.

(2S)-1,4-dioxane-2-methanol in step 6) was replaced with an equimolar amount of 4-hydroxymethyltetrahydropyran to obtain compound 46, a white solid, with a one-step yield of 48% in step 6).

¹H NMR (600 MHz, Chloroform-d) δ 8.75 (dd, J=4.8, 1.8 Hz, 1H), 8.60 (d, J=2.4 Hz, 1H), 7.81-7.71 (m, 1H), 7.47 (s, 1H), 7.34 (dd, J=8.4, 4.8 Hz, 1H), 7.01 (t, J=8.4 Hz, 1H), 6.65 (dd, J=8.4, 2.4 Hz, 1H), 6.55 (dd, J=11.4, 2.4 Hz, 1H), 6.30 (d, J=1.8 Hz, 1H), 4.08-3.99 (m, 2H), 3.81 (d, J=6.6 Hz, 2H), 3.76 (s, 2H), 3.52-3.41 (m, 3H), 2.81-2.72 (m, 2H), 2.13-2.04 (m, 1H), 1.81-1.72 (m, 2H), 1.53-1.42 (m, 2H), 1.21 (t, J=7.2 Hz, 3H).

### Preparation Example 47: The Preparation Example is Used to Describe the Synthesis Process of Hydrochloride Salt of Compound 47

The preparation method of this preparation example is similar to that of preparation example 1, with the following differences:

(2S)-1,4-dioxane-2-methanol in step 6) was replaced with an equimolar amount of 1-(tetrahydro-2H-pyran-4-yl)ethanol to obtain hydrochloride salt of compound 47, a white solid, with a one-step yield of 38% in step 6).

¹H NMR (600 MHz, Methanol-d₄) δ 8.86 (dd, J=4.8, 1.6 Hz, 1H), 8.62 (d, J=2.4 Hz, 1H), 8.04-7.98 (m, 1H), 7.81 (d, J=1.8 Hz, 1H), 7.65 (dd, J=8.4, 4.8 Hz, 1H), 7.01 (t, J=8.4 Hz, 1H), 6.76 (dd, J=8.4, 2.4 Hz, 1H), 6.67 (dd, J=12.0, 2.4 Hz, 1H), 6.41 (d, J=1.8 Hz, 1H), 4.35-4.29 (m, 1H), 4.10 (s, 2H), 4.01 (dd, J=11.8, 4.2 Hz, 2H), 3.50-3.40 (m, 2H), 2.72 (s, 3H), 1.92-1.80 (m, 2H), 1.70-1.62 (m, 1H), 1.58-1.42 (m, 2H), 1.32 (d, J=6.0 Hz, 3H).

### Preparation Example 48: The Preparation Example is Used to Describe the Synthesis Process of Compound 48

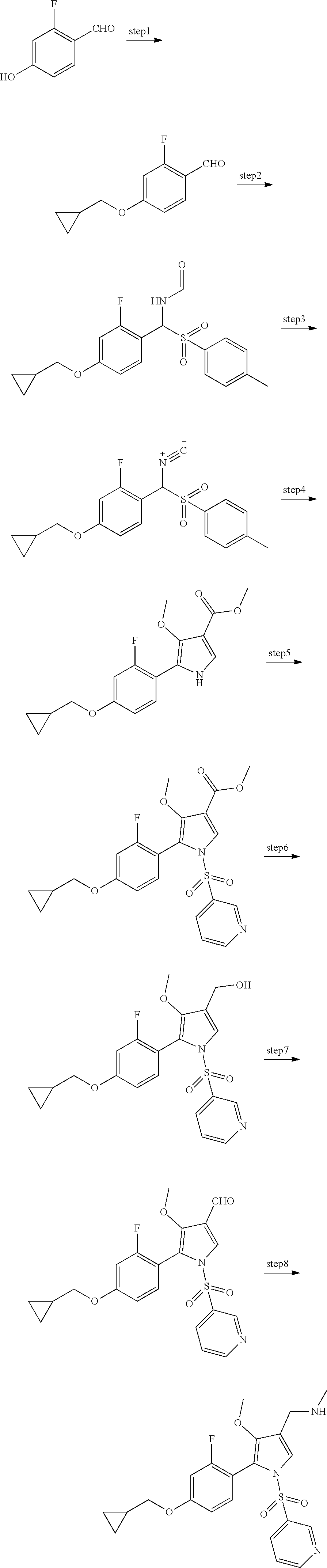

### Step 1: preparation of 4-(cyclopropylmethoxy)-2-fluorobenzaldehyde

2-fluoro-4-hydroxybenzaldehyde (14.2 mmol), (bromomethyl)cyclopropane (20.7 mmol), potassium carbonate (42.7 mmol) and DMF (40 mL) were added to a one-neck round-bottom flask and stirred at 60° C. overnight. After it was detected by TLC that the raw materials were fully reacted, the system was purified by column chromatography to obtain 2.7 g of white oily liquid, with a yield of 99%.

### Step 2: preparation of N-((4-(cyclopropylmethoxy)-2-fluorophenyl)(tolylsulfonyl) methyl)formamide

Sodium 4-methylbenzenesulfonate (32.0 mmol), (15)-(+)-10-camphorsulfonic acid (0.320 mmol), 4-(cyclopropylmethoxy)-2-fluorobenzaldehyde (35.2 mmol) and formamide (160 mmol) were added to a round-bottom flask, and the reaction mixture was stirred at about 65° C. for about 16 h and solidified overnight. The solid was decomposed and suspended in methanol. The solid block was ground into powder by shovel, filtered and washed with methanol. The filter cake was dried under vacuum at about 60° C., and the compound obtained was white powder, with a yield of 79%.

### Step 3: preparation of 4-(cyclopropylmethoxy)-2-fluoro-1-(isocyano(tolylsulfonyl) methyl)benzene

N-((4-(cyclopropylmethoxy)-2-fluorophenyl)(tolylsulfonyl)methyl)formamide (105.2 mmol) was dissolved in 500 ml of anhydrous tetrahydrofuran, phosphorous oxychloride (210.4 mmol) was added to a reaction flask, and the system was cooled in an ice bath under nitrogen protection, triethylamine (631.1 mmol) was added dropwise at 0-10° C., and after the addition, the system was stirred in an ice bath for 60 min. Treatment was conducted after it was by TLC that the raw materials disappeared: 500 mL of water was added dropwise for quenching, MTBE was used for extraction (200 mL×3), and the organic phases were merged, washed with 300 mL of saturated brine×2, dried with anhydrous sodium sulfate, filtered and the solvent was removed by rotary evaporation under reduced pressure and obtain a yellow semi-solid. 120 mL of petroleum ether and 6 mL of ethyl acetate were added, stirred at room temperature for 30 min and filtered. The product was blown dry at room temperature to obtain a light yellow solid, with a yield of 72%.

### Step 4: preparation of 5-(4-(cyclopropylmethoxy)-2-fluorophenyl)-4-methoxy-1H-pyrrole-3-methyl carboxylate

NaH (67.5 mmol) and 250 mL of diethyl ether were cooled in an ice bath under nitrogen protection, and 4-(cyclopropylmethoxy)-2-fluoro-1-(isocyano(tolylsulfonyl)methyl)benzene (225.0 mmol) and 2-(methoxymethylene)dimethyl malonate (13.5 mmol) dissolved in 250 mL of acetonitrile were added dropwise at −10° C. After the addition, the system was heated up to 40° C. and stirred for 48 h. After the reaction, 500 mL of water was added, and the system was extracted with 100 ml of MTBE for three times, the organic phases were merged, washed with 300 mL of saturated brine, dried with anhydrous sodium sulfate, filtered and the solvent was removed by rotary evaporation under reduced pressure and obtain an oily substance, 100-200 silica gel 1.5 times the weight was added to form sand, 200-300 silica gel 4 times the weight was loaded into the column, column chromatography was performed, and the produce was collected and concentrated under reduced pressure and then 50 mL of n-heptane and 2 mL of ethyl acetate were added at room temperature, pulped for 1 h and filtered to obtain a yellow powdery solid, with a yield of 45%.

### Step 5: preparation of 5-(4-(cyclopropylmethoxy)-2-fluorophenyl)-4-methoxy-1-(pyridin-3-ylsulfonyl)-1H-pyrrole-3-methyl carboxylate

60 mL of ultra-dry tetrahydrofuran was added to a one-neck round-bottom flask and cooled to 0° C., NaH (60% in kerosene, 47.2 mmol) was added, 5-(4-(cyclopropylmethoxy)-2-fluorophenyl)-4-methoxy-1H-pyrrole-3-methyl carboxylate (13.5 mmol) dissolved in 10 mL of N,N-dimethylformamide was added dropwise slowly, they were stirred at 0° C. for 30 min, then 15-crown-5 (40.5 mmol) was added dropwise, and then pyridine-3-sulfonyl chloride (20.2 mmol) was added dropwise. After the addition, the system was further stirred at 0° C. for 30 min and then stirred at room temperature for 30 min. After it was detected by TLC that the raw materials were fully reacted, the system was poured into ice water and extracted with ethyl acetate (200 mL×2) and washed with saturated brine (150 mL×2), and the organic phases were merged, dried with anhydrous sodium sulfate, the organic phase was concentrated under reduced pressure and then purified by column chromatography to obtain a light yellow solid, with a yield of 71.5%.

### Step 6: preparation of (5-(4-(cyclopropylmethoxy)-2-fluorophenyl)-4-methoxy-1-(pyridin-3-ylsulfonyl)-1H-pyrrol-3-yl)methanol

5-(4-(cyclopropylmethoxy)-2-fluorophenyl)-4-methoxy-1-(pyridin-3-ylsulfonyl)-1H-pyrrole-3-methyl carboxylate (60.8 mmol) was dissolved in tetrahydrofuran. Diisobutylaluminum hydride (1.0M n-hexane solution) (182.2 mmol) was added to the obtained solution at 0° C., and then stirred at room temperature for 1 h. Water was added to the reaction mixture at 0° C., and the solution was extracted with ethyl acetate. The extract was dried with anhydrous magnesium sulfate and then concentrated under reduced pressure. The obtained residue was purified by silica gel column chromatography (ethyl acetate:n-hexane=1:4(v/v)) to obtain a yellow compound, with a yield of 50%.

### Step 7: preparation of 5-(4-(cyclopropylmethoxy)-2-fluorophenyl)-4-methoxy-1-(pyridin-3-ylsulfonyl)-1H-pyrrolo-3-formaldehyde

(5-(4-(cyclopropylmethoxy)-2-fluorophenyl)-4-methoxy-1-(pyridin-3-ylsulfonyl)-1H-pyrrolo-3-yl)methanol (50.0 mmol) was dissolved in dichloromethane. Pyridinium chlorochromate (150.0 mmol) was added to the obtained solution and they were stirred at room temperature for 30 min and filtered with a diatomite earth mat. The filtrate was concentrated under reduced pressure. The obtained residue was purified by silica gel column chromatography to obtain a yellow compound, with a yield of 63.5%.

### Step 8: preparation of 1-(5-(4-(cyclopropylmethoxy)-2-fluorophenyl)-4-methoxy-1-(pyridin-3-ylsulfonyl)-1H-pyrrolo-3-yl)-N-methylmethylamine

5-(4-(cyclopropylmethoxy)-2-fluorophenyl)-4-methoxy-1-(pyridin-3-ylsulfonyl)-1H-pyrrolo-3-formaldehyde (0.23 mmol), methylamine (33 wt % methanol solution, 2.3 mmol) and methanol (3 mL) were added into a one-neck round-bottom flask in an ice-water bath and stirred for 30 min. The ice-water bath was removed and they were heated up naturally to the ambient temperature and stirred and reacted overnight. Next day, sodium borohydride (1.2 mmol) was added to further react at room temperature (25° C.) for 3 h. After it was detected by TLC that the raw materials were fully reacted, the system was added to ice water, extracted with dichloromethane (50 mL×4), dried with anhydrous sodium sulfate and the organic phase was concentrated under reduced pressure and purified by column chromatography to obtain a white solid, with a one-step yield of 33%.

¹H NMR (600 MHz, Methanol-d₄) δ 8.89 (d, J=4.8 Hz, 1H), 8.60 (s, 1H), 8.07 (d, J=7.8 Hz, 1H), 7.80-7.67 (m, 2H), 7.02 (t, J=8.4 Hz, 1H), 6.76 (dd, J=8.4, 2.4 Hz, 1H), 6.63 (dd, J=11.4, 2.4 Hz, 1H), 4.08 (s, 2H), 3.88 (d, J=6.6 Hz, 2H), 3.44 (s, 3H), 2.72 (s, 3H), 1.32-1.28 (m, 1H), 0.69-0.61 (m, 2H), 0.46-0.31 (m, 2H).

### Preparation Example 49: The Preparation Example is Used to Describe the Synthesis Process of Hydrochloride Salt of Compound 49

The preparation method of this preparation example is similar to that of preparation example 1, with the following differences:

(2S)-1,4-dioxane-2-methanol in step 6) was replaced with an equimolar amount of (S)-glycidol to obtain hydrochloride salt of compound 49, a grey solid, with a one-step yield of 45% in step 6).

¹H NMR (400 MHz, Methanol-d₄) δ 8.82 (d, J=4.8 Hz, 1H), 8.57 (s, 1H), 7.92 (d, J=8.0 Hz, 1H), 7.79 (s, 1H), 7.59 (dd, J=8.4, 4.8 Hz, 1H), 7.02 (t, J=8.4 Hz, 1H), 6.85-6.77 (m, 1H), 6.72 (dd, J=11.6, 2.4 Hz, 1H), 6.40 (d, J=2.0 Hz, 1H), 4.20-4.06 (m, 5H), 3.82-3.75 (m, 1H), 3.75-3.67 (m, 1H), 2.71 (s, 3H).

### Preparation Example 50: The Preparation Example is Used to Describe the Synthesis Process of Hydrochloride Salt of Compound 50

The preparation method of this preparation example is similar to that of preparation example 1, with the following differences:

(2S)-1,4-dioxane-2-methanol in step 6) was replaced with an equimolar amount of (R)-glycidol to obtain hydrochloride salt of compound 50, a grey solid, with a one-step yield of 46% in step 6).

¹H NMR (400 MHz, Methanol-d₄) δ 8.89 (d, J=5.2 Hz, 1H), 8.67 (s, 1H), 8.13-8.05 (m, 1H), 7.82 (d, J=2.0 Hz, 1H), 7.72 (dd, J=8.4, 5.2 Hz, 1H), 7.04 (t, J=8.4 Hz, 1H), 6.82 (dd, J=8.4, 2.4 Hz, 1H), 6.74 (dd, J=11.2, 2.4 Hz, 1H), 6.43 (d, J=1.6 Hz, 1H), 4.20-4.07 (m, 5H), 3.78 (dd, J=11.2, 4.8 Hz, 1H), 3.71 (dd, J=11.2, 5.2 Hz, 1H), 2.71 (s, 3H).

### Preparation Example 51: The Preparation Example is Used to Describe the Synthesis Process of Hydrochloride Salt of Compound 51

The preparation method of this preparation example is similar to that of preparation example 48, with the following differences:

5-(4-(cyclopropylmethoxy)-2-fluorophenyl)-4-methoxy-1H-pyrrole-3-methyl carboxylate in step 4) was replaced with an equimolar amount of 5-(4-(cyclopropylmethoxy)-2-fluorophenyl)-4-fluoro-1H-pyrrole-3-methyl carboxylate to obtain hydrochloride salt of compound 51, a brown solid, with a one-step yield of 46% in step 6).

¹H NMR (600 MHz, Methanol-d₄) δ 8.90 (d, J=5.4 Hz, 1H), 8.70 (d, J=2.4 Hz, 1H), 8.20 (d, J=9.0 Hz, 1H), 7.85-7.79 (m, 2H), 7.04 (t, J=8.4 Hz, 1H), 6.82-6.78 (m, 1H), 6.49-6.44 (m, 1H), 4.10 (s, 2H), 3.84 (s, 2H), 2.73 (s, 3H), 1.33 (s, 6H).

### Preparation Example 52: The Preparation Example is Used to Describe the Synthesis Process of Hydrochloride Salt of Compound 52

The preparation method of this preparation example is similar to that of preparation example 1, with the following differences:

(2S)-1,4-dioxane-2-methanol in step 6) was replaced with an equimolar amount of 2-methylpropane-1,2-diol to obtain hydrochloride salt of compound 52, a brown solid, with a one-step yield of 46% in step 6).

¹H NMR (600 MHz, Methanol-d₄) δ 8.94 (d, J=5.4 Hz, 1H), 8.72 (d, J=2.4 Hz, 1H), 8.20 (d, J=9.0 Hz, 1H), 7.89-7.79 (m, 2H), 7.03 (t, J=8.4 Hz, 1H), 6.86-6.79 (m, 1H), 6.77-6.70 (m, 1H), 6.48-6.42 (m, 1H), 4.11 (s, 2H), 3.86 (s, 2H), 2.72 (s, 3H), 1.34 (s, 6H).

### Preparation Example 53: The Preparation Example is Used to Describe the Synthesis Process of Hydrochloride Salt of Compound 53

The preparation method of this preparation example is similar to that of preparation example 1, with the following differences:

(2S)-1,4-dioxane-2-methanol in step 6) was replaced with an equimolar amount of cis-2-fluorocyclopropyl)methanol to obtain hydrochloride salt of compound 53, a white solid, with a one-step yield of 46% in step 6).

¹H NMR (600 MHz, Methanol-d₄) δ 8.93 (d, J=5.2 Hz, 1H), 8.75-8.66 (m, 1H), 8.16 (d, J=7.8 Hz, 1H), 7.88-7.75 (m, 2H), 7.03 (dd, J=9.5, 7.6 Hz, 1H), 6.81 (dd, J=8.6, 2.5 Hz, 1H), 6.72 (dd, J=11.4, 2.5 Hz, 1H), 6.44 (d, J=2.1 Hz, 1H), 4.34-4.25 (m, 1H), 4.17-4.02 (m, 3H), 2.74 (s, 3H), 1.05-0.97 (m, 1H), 0.95-0.81 (m, 1H).

### Preparation Example 54: The Preparation Example is Used to Describe the Synthesis Process of Compound 54

The preparation method of this preparation example is similar to that of preparation example 1, with the following differences:

(2S)-1,4-dioxane-2-methanol in step 6) was replaced with an equimolar amount of 2,5-difluorobenzyl alcohol to obtain compound 54, a white solid, with a one-step yield of 48% in step 6).

¹H NMR (600 MHz, Chloroform-d) δ 8.74 (d, J=4.8 Hz, 1H), 8.63 (s, 1H), 7.71 (d, J=8.4 Hz, 1H), 7.38 (s, 1H), 7.32 (dd, J=8.4, 4.8 Hz, 1H), 7.25-7.21 (m, 1H), 7.10-7.05 (m, 2H), 7.04-6.98 (m, 1H), 6.75 (d, J=8.4 Hz, 1H), 6.66 (d, J=11.4 Hz, 1H), 6.24 (s, 1H), 5.12 (s, 2H), 3.63 (s, 2H), 2.45 (s, 3H).

### Preparation Example 55: The Preparation Example is Used to Describe the Synthesis Process of Hydrochloride Salt of Compound 55

The preparation method of this preparation example is similar to that of preparation example 1, with the following differences:

(2S)-1,4-dioxane-2-methanol in step 6) was replaced with an equimolar amount of 2,4-difluorobenzyl alcohol to obtain hydrochloride salt of compound 55, a yellow oily substance, with a one-step yield of 45% in step 6).

¹H NMR (600 MHz, Methanol-d₄) δ 8.85-8.79 (m, 1H), 8.57 (s, 1H), 7.91 (d, J=8.4 Hz, 1H), 7.80 (s, 1H), 7.64-7.53 (m, 2H), 7.10-7.00 (m, 3H), 6.86 (d, J=8.4 Hz, 1H), 6.79 (d, J=11.4 Hz, 1H), 6.40 (d, J=2.4 Hz, 1H), 5.18 (s, 2H), 4.10 (s, 2H), 2.72 (s, 3H).

### Preparation Example 56: The Preparation Example is Used to Describe the Synthesis Process of Hydrochloride Salt of Compound 56

The preparation method of this preparation example is similar to that of preparation example 1, with the following differences:

(2S)-1,4-dioxane-2-methanol in step 6) was replaced with an equimolar amount of 2,6-difluorobenzyl alcohol to obtain hydrochloride salt of compound 56, a white solid, with a one-step yield of 45% in step 6).

¹H NMR (600 MHz, Methanol-d₄) δ 8.85 (d, J=4.8 Hz, 1H), 8.63 (s, 1H), 7.97 (d, J=8.4 Hz, 1H), 7.81 (s, 1H), 7.62 (t, J=6.6 Hz, 1H), 7.54-7.43 (m, 1H), 7.15-6.98 (m, 3H), 6.87 (d, J=8.4 Hz, 1H), 6.81 (d, J=11.4 Hz, 1H), 6.42 (s, 1H), 5.21 (s, 2H), 4.10 (s, 2H), 2.72 (s, 3H).

### Preparation Example 57: The Preparation Example is Used to Describe the Synthesis Process of Hydrochloride Salt of Compound 57

The preparation method of this preparation example is similar to that of preparation example 1, with the following differences:

(2S)-1,4-dioxane-2-methanol in step 6) was replaced with an equimolar amount of 3,4-difluorobenzyl alcohol to obtain hydrochloride salt of compound 57, a white solid, with a one-step yield of 56% in step 6).

¹H NMR (600 MHz, Methanol-d₄) δ 8.85 (d, J=4.8 Hz, 1H), 8.60 (d, J=2.4 Hz, 1H), 7.99 (d, J=8.4 Hz, 1H), 7.81 (s, 1H), 7.67-7.60 (m, 1H), 7.43 (t, J=9.6 Hz, 1H), 7.31 (m, 2H), 7.04 (t, J=8.4 Hz, 1H), 6.86 (d, J=9.0 Hz, 1H), 6.78 (d, J=11.4 Hz, 1H), 6.42 (d, J=2.1 Hz, 1H), 5.14 (s, 2H), 4.10 (s, 2H), 2.71 (s, 4H).

### Preparation Example 58: The Preparation Example is Used to Describe the Synthesis Process of Hydrochloride Salt of Compound 58

The preparation method of this preparation example is similar to that of preparation example 1, with the following differences:

(2S)-1,4-dioxane-2-methanol in step 6) was replaced with an equimolar amount of 3,5-difluorobenzyl alcohol to obtain hydrochloride salt of compound 58, a white solid, with a one-step yield of 55% in step 6).

¹H NMR (600 MHz, Methanol-d₄) δ 8.84 (d, J=4.8 Hz, 1H), 8.59 (s, 1H), 7.94 (d, J=8.4 Hz, 1H), 7.82 (s, 1H), 7.64-7.55 (m, 1H), 7.16-7.10 (m, 2H), 7.06 (t, J=8.4 Hz, 1H), 6.99-6.93 (m, 1H), 6.88 (d, J=8.4 Hz, 1H), 6.81 (d, J=11.4 Hz, 1H), 6.43 (d, J=2.4 Hz, 1H), 5.21 (s, 2H), 4.12 (s, 2H), 2.73 (s, 3H).

### Preparation Example 59: The Preparation Example is Used to Describe the Synthesis Process of Compound 59

The preparation method of this preparation example is similar to that of preparation example 1, with the following differences:

(2S)-1,4-dioxane-2-methanol in step 6) was replaced with an equimolar amount of 2-difluoromethoxybenzyl alcohol to obtain compound 59, yellow powder, with a one-step yield of 49% in step 6).

¹H NMR (400 MHz, Methanol-d₄) δ 8.75 (dd, J=4.8, 1.6 Hz, 1H), 8.53 (d, J=2.4 Hz, 1H), 7.87-7.81 (m, 1H), 7.58 (dd, J=7.6, 1.6 Hz, 1H), 7.51 (d, J=1.8 Hz, 1H), 7.50-7.46 (m, 1H), 7.42 (td, J=7.6, 1.6 Hz, 1H), 7.34-7.22 (m, 2H), 7.01 (t, J=8.4 Hz, 1H), 6.88 (t, J=73.6 Hz, 1H), 6.81 (dd, J=8.4, 2.4 Hz, 1H), 6.74 (dd, J=11.2, 2.4 Hz, 1H), 6.29 (d, J=1.6 Hz, 1H), 5.19 (s, 2H), 3.63 (s, 2H), 2.39 (s, 3H).

### Preparation Example 60: The Preparation Example is Used to Describe the Synthesis Process of Hydrochloride Salt of Compound 60

The preparation method of this preparation example is similar to that of preparation example 1, with the following differences:

(2S)-1,4-dioxane-2-methanol in step 6) was replaced with an equimolar amount of 2-trifluoromethoxybenzyl alcohol to obtain hydrochloride salt of compound 60, a white solid, with a one-step yield of 56% in step 6).

¹H NMR (400 MHz, Methanol-d₄) δ 8.88 (d, J=5.2 Hz, 1H), 8.67 (s, 1H), 8.04 (ddd, J=8.4, 2.4, 1.6 Hz, 1H), 7.82 (d, J=2.0 Hz, 1H), 7.71-7.63 (m, 2H), 7.52-7.46 (m, 1H), 7.45-7.36 (m, 2H), 7.05 (t, J=8.4 Hz, 1H), 6.85 (dd, J=8.4, 2.4 Hz, 1H), 6.77 (dd, J=11.2, 2.4 Hz, 1H), 6.44 (d, J=2.0 Hz, 1H), 5.23 (s, 2H), 4.11 (s, 2H), 2.72 (s, 3H).

### Preparation Example 61: The Preparation Example is Used to Describe the Synthesis Process of Hydrochloride Salt of Compound 61

The preparation method of this preparation example is similar to that of preparation example 1, with the following differences:

(2S)-1,4-dioxane-2-methanol in step 6) was replaced with an equimolar amount of 4-difluoromethoxybenzyl alcohol to obtain hydrochloride salt of compound 61, a white solid, with a one-step yield of 55% in step 6).

¹H NMR (600 MHz, Methanol-d₄) δ 8.87-8.80 (m, 1H), 8.60 (d, J=2.4 Hz, 1H), 7.96 (d, J=7.8 Hz, 1H), 7.81 (s, 1H), 7.64-7.57 (m, 1H), 7.56-7.51 (m, 2H), 7.24-7.18 (m, 2H), 7.02 (t, J=8.4 Hz, 1H), 6.86 (t, J=66.0 Hz, 1H), 6.86-6.84 (m, 1H), 6.79-6.75 (m, 1H), 6.41 (d, J=2.4 Hz, 1H), 5.16 (s, 2H), 4.10 (s, 2H), 2.72 (s, 3H).

### Preparation Example 62: The Preparation Example is Used to Describe the Synthesis Process of Hydrochloride Salt of Compound 62

The preparation method of this preparation example is similar to that of preparation example 1, with the following differences:

(2S)-1,4-dioxane-2-methanol in step 6) was replaced with an equimolar amount of 1-hydroxymethylnaphthalene to obtain hydrochloride salt of compound 62, a white solid, with a one-step yield of 53% in step 6).

¹H NMR (600 MHz, Methanol-d₄) δ8.83-8.78 (m, 1H), 8.61 (s, 1H), 8.13 (d, J=8.4 Hz, 1H), 7.98-7.84 (m, 3H), 7.80 (s, 1H), 7.67 (d, J=6.6 Hz, 1H), 7.64-7.46 (m, 3H), 7.04 (t, J=8.4 Hz, 1H), 6.92 (d, J=8.4 Hz, 1H), 6.85 (d, J=11.4 Hz, 1H), 6.41 (s, 1H), 5.62 (s, 2H), 4.10 (s, 2H), 2.72 (s, 3H).

### Preparation Example 63: The Preparation Example is Used to Describe the Synthesis Process of Hydrochloride Salt of Compound 63

The preparation method of this preparation example is similar to that of preparation example 1, with the following differences:

Pyridine-3-sulfonyl chloride in step 3) was replaced with an equimolar amount of 3-cyano-benzenesulfonyl chloride, and (2S)-1,4-dioxane-2-methanol in step 6) was replaced with an equimolar amount of 1-hydroxymethylnaphthalene to obtain hydrochloride salt of compound 63, a white solid, with a one-step yield of 43% in step 6).

¹H NMR (600 MHz, Methanol-d₄) δ 8.19-8.12 (m, 1H), 8.05-8.00 (m, 1H), 7.94 (dd, J=12.6, 8.4 Hz, 2H), 7.84-7.73 (m, 2H), 7.72-7.66 (m, 2H), 7.65-7.48 (m, 4H), 7.09-7.01 (m, 1H), 6.98-6.91 (m, 1H), 6.89-6.83 (m, 1H), 6.41 (s, 1H), 5.63 (s, 2H), 4.11 (s, 2H), 2.73 (s, 3H).

### Preparation Example 64: The Preparation Example is Used to Describe the Synthesis Process of Hydrochloride Salt of Compound 64

The preparation method of this preparation example is similar to that of preparation example 1, with the following differences:

(2S)-1,4-dioxane-2-methanol in step 6) was replaced with an equimolar amount of 4-hydroxymethylbenzoic acid to obtain hydrochloride salt of compound 64, a white solid, with a one-step yield of 25% in step 6).

¹H NMR (600 MHz, Methanol-d₄) δ 8.85 (d, J=5.4 Hz, 1H), 8.63 (s, 1H), 8.07 (d, J=7.8 Hz, 2H), 8.00 (d, J=7.2 Hz, 1H), 7.80 (s, 1H), 7.68-7.52 (m, 3H), 7.08-6.97 (m, 1H), 6.86 (d, J=8.4 Hz, 1H), 6.78 (d, J=11.4 Hz, 1H), 6.41 (s, 1H), 5.25 (s, 2H), 4.09 (s, 2H), 2.71 (s, 3H).

### Preparation Example 65: The Preparation Example is Used to Describe the Synthesis Process of Hydrochloride Salt of Compound 65

The preparation method of this preparation example is similar to that of preparation example 1, with the following differences:

(2S)-1,4-dioxane-2-methanol in step 6) was replaced with an equimolar amount of 1-(2,5-difluorophenyl)ethanol to obtain hydrochloride salt of compound 65, a white solid, with a one-step yield of 48% in step 6).

¹H NMR (600 MHz, Methanol-d₄) δ 8.84 (d, J=4.8 Hz, 1H), 8.61 (d, J=2.4 Hz, 1H), 7.93 (d, J=8.4 Hz, 1H), 7.79 (s, 1H), 7.63-7.56 (m, 1H), 7.23-7.16 (m, 2H), 7.11-7.05 (m, 1H), 6.98 (t, J=8.4 Hz, 1H), 6.73 (dd, J=8.4, 2.4 Hz, 1H), 6.63 (dd, J=11.4, 2.4 Hz, 1H), 6.39 (s, 1H), 5.76 (q, J=6.4 Hz, 1H), 4.08 (s, 2H), 2.70 (s, 3H), 1.68 (d, J=6.4 Hz, 3H).

### Preparation Example 66: The Preparation Example is Used to Describe the Synthesis Process of Hydrochloride Salt of Compound 66

The preparation method of this preparation example is similar to that of preparation example 1, with the following differences:

(2S)-1,4-dioxane-2-methanol in step 6) was replaced with an equimolar amount of (s)-1-(2,6-dichloro-3-fluorophenyl)ethanol to obtain hydrochloride salt of compound 66, a white solid, with a one-step yield of 56% in step 6).

¹H NMR (600 MHz, Methanol-d₄) δ 8.89 (dd, J=5.4, 1.8 Hz, 1H), 8.65 (d, J=2.4 Hz, 1H), 8.02-7.97 (m, 1H), 7.81 (d, J=1.8 Hz, 1H), 7.70-7.65 (m, 1H), 7.50-7.45 (m, 1H), 7.28 (t, J=8.4 Hz, 1H), 6.98 (t, J=8.4 Hz, 1H), 6.69 (dd, J=8.4, 2.4 Hz, 1H), 6.59 (dd, J=11.4, 2.4 Hz, 1H), 6.40 (d, J=1.8 Hz, 1H), 6.16 (q, J=6.6 Hz, 1H), 4.10 (s, 2H), 2.71 (s, 3H), 1.84 (d, J=6.6 Hz, 3H).

### Preparation Example 67: The Preparation Example is Used to Describe the Synthesis Process of Hydrochloride Salt of Compound 67

The preparation method of this preparation example is similar to that of preparation example 1, with the following differences:

(2S)-1,4-dioxane-2-methanol in step 6) was replaced with an equimolar amount of 1-(2,6-difluorophenyl)ethanol to obtain hydrochloride salt of compound 67, a white solid, with a one-step yield of 53% in step 6).

¹H NMR (600 MHz, Methanol-d₄) δ 8.92-8.88 (m, 1H), 8.68 (d, J=2.4 Hz, 1H), 8.04-7.97 (m, 1H), 7.82 (d, J=1.8 Hz, 1H), 7.67 (dd, J=8.4, 5.4 Hz, 1H), 7.45-7.32 (m, 1H), 7.02 (t, J=8.4 Hz, 2H), 6.98 (t, J=8.4 Hz, 1H), 6.76 (dd, J=8.4, 2.4 Hz, 1H), 6.65 (dd, J=11.4, 2.4 Hz, 1H), 6.40 (d, J=2.4 Hz, 1H), 5.88 (q, J=6.6 Hz, 1H), 4.10 (s, 2H), 2.72 (s, 3H), 1.81 (d, J=6.6 Hz, 3H).

### Preparation Example 68: The Preparation Example is Used to Describe the Synthesis Process of Hydrochloride Salt of Compound 68

The preparation method of this preparation example is similar to that of preparation example 1, with the following differences:

(2S)-1,4-dioxane-2-methanol in step 6) was replaced with an equimolar amount of 1-(3,5-difluorophenyl)ethanol to obtain hydrochloride salt of compound 68, a white solid, with a one-step yield of 55% in step 6).

¹H NMR (400 MHz, Methanol-d₄) δ 8.84 (dd, J=4.8, 1.6 Hz, 1H), 8.60 (d, J=2.4 Hz, 1H), 7.93 (dt, J=8.4, 2.0 Hz, 1H), 7.79 (d, J=1.6 Hz, 1H), 7.59 (dd, J=8.4, 4.8 Hz, 1H), 7.10-7.01 (m, 2H), 6.96 (t, J=8.4 Hz, 1H), 6.87 (tt, J=9.2, 2.4 Hz, 1H), 6.74 (dd, J=8.4, 2.4 Hz, 1H), 6.65 (dd, J=11.2, 2.4 Hz, 1H), 6.38 (d, J=2.0 Hz, 1H), 5.54 (q, J=6.4 Hz, 1H), 4.08 (s, 2H), 2.70 (s, 3H), 1.64 (d, J=6.4 Hz, 3H).

### Preparation Example 69: The Preparation Example is Used to Describe the Synthesis Process of Hydrochloride Salt of Compound 69

The preparation method of this preparation example is similar to that of preparation example 1, with the following differences:

Pyridine-3-sulfonyl chloride in step 3) was replaced with an equimolar amount of 3-cyano-benzenesulfonyl chloride, and (2S)-1,4-dioxane-2-methanol in step 6) was replaced with an equimolar amount of 1-(2,6-difluorophenyl)ethanol to obtain hydrochloride salt of compound 69, a white solid, with a one-step yield of 43% in step 6).

¹H NMR (400 MHz, Methanol-d₄) δ 8.00 (dt, J=7.6, 1.6 Hz, 1H), 7.76 (d, J=2.0 Hz, 1H), 7.70-7.65 (m, 1H), 7.64-7.53 (m, 2H), 7.42-7.32 (m, 1H), 7.00 (t, J=8.4 Hz, 2H), 6.93 (t, J=8.4 Hz, 1H), 6.74 (dd, J=8.8, 2.4 Hz, 1H), 6.60 (dd, J=11.2, 2.4 Hz, 1H), 6.35 (d, J=2.0 Hz, 1H), 5.87 (q, J=6.4 Hz, 1H), 4.07 (s, 2H), 2.70 (s, 3H), 1.80 (d, J=6.4 Hz, 3H).

### Preparation Example 70: The Preparation Example is Used to Describe the Synthesis Process of Hydrochloride Salt of Compound 70

The preparation method of this preparation example is similar to that of preparation example 1, with the following differences:

Pyridine-3-sulfonyl chloride in step 3) was replaced with an equimolar amount of 3-cyano-benzenesulfonyl chloride, and (2S)-1,4-dioxane-2-methanol in step 6) was replaced with an equimolar amount of 1-(3,5-difluorophenyl)ethanol to obtain hydrochloride salt of compound 70, a white solid, with a one-step yield of 45% in step 6).

¹H NMR (400 MHz, Methanol-d₄) δ 8.01 (dt, J=7.6, 1.2 Hz, 1H), 7.76 (d, J=2.0 Hz, 1H), 7.71-7.67 (m, 1H), 7.64-7.58 (m, 2H), 7.11-7.01 (m, 2H), 6.94 (t, J=8.4 Hz, 1H), 6.86 (tt, J=9.2, 2.4 Hz, 1H), 6.74 (dd, J=8.4, 2.4 Hz, 1H), 6.63 (dd, J=11.2, 2.4 Hz, 1H), 6.35 (d, J=2.0 Hz, 1H), 5.54 (q, J=6.4 Hz, 1H), 4.07 (s, 2H), 2.69 (s, 3H), 1.64 (d, J=6.4 Hz, 3H).

### Preparation Example 71: The Preparation Example is Used to Describe the Synthesis Process of Hydrochloride Salt of Compound 71

The preparation method of this preparation example is similar to that of preparation example 1, with the following differences:

Pyridine-3-sulfonyl chloride in step 3) was replaced with an equimolar amount of 3-(cyclopropylmethoxy)benzenesulfonyl chloride, and (2S)-1,4-dioxane-2-methanol in step 6) was replaced with an equimolar amount of 1-(3,5-difluorophenyl)ethanol to obtain hydrochloride salt of compound 71, a white solid, with a one-step yield of 47% in step 6).

¹H NMR (600 MHz, Methanol-d₄) δ 7.71 (d, J=1.8 Hz, 1H), 7.34-7.24 (m, 1H), 7.15 (dd, J=8.4, 2.4 Hz, 1H), 7.09-7.04 (m, 2H), 6.98-6.94 (m, 1H), 6.94-6.85 (m, 2H), 6.81 (t, J=2.4 Hz, 1H), 6.70 (dd, J=8.4, 2.4 Hz, 1H), 6.63 (dd, J=11.4, 2.4 Hz, 1H), 6.31 (d, J=2.4 Hz, 1H), 5.53 (q, J=6.6 Hz, 1H), 4.06 (s, 2H), 3.81-3.65 (m, 2H), 2.68 (s, 3H), 1.64 (d, J=6.6 Hz, 3H), 1.26-1.17 (m, 1H), 0.67-0.59 (m, 2H), 0.39-0.32 (m, 2H).

### Preparation Example 72: The Preparation Example is Used to Describe the Synthesis Process of Hydrochloride Salt of Compound 72

The preparation method of this preparation example is similar to that of preparation example 1, with the following differences:

Pyridine-3-sulfonyl chloride in step 3) was replaced with an equimolar amount of 3-(cyclopropylmethoxy)benzenesulfonyl chloride, and (2S)-1,4-dioxane-2-methanol in step 6) was replaced with an equimolar amount of 1-(2,6-difluorophenyl)ethanol to obtain hydrochloride salt of compound 72, a yellow oily substance, with a one-step yield of 45% in step 6).

¹H NMR (400 MHz, Methanol-d₄) δ 7.67 (d, J=2.0 Hz, 1H), 7.44-7.31 (m, 1H), 7.24 (t, J=8.0 Hz, 1H), 7.14 (dd, J=8.4, 2.4 Hz, 1H), 7.04-6.97 (m, 2H), 6.94-6.85 (m, 2H), 6.81 (t, J=2.0 Hz, 1H), 6.69 (dd, J=8.4, 2.4 Hz, 1H), 6.59 (dd, J=11.4, 2.4 Hz, 1H), 6.27 (d, J=2.0 Hz, 1H), 5.85 (q, J=6.4 Hz, 1H), 4.00 (s, 2H), 3.73 (d, J=7.2 Hz, 2H), 2.65 (s, 3H), 1.80 (d, J=6.4 Hz, 3H), 1.28-1.11 (m, 1H), 0.71-0.54 (m, 2H), 0.42-0.27 (m, 2H).

### Preparation Example 73: The Preparation Example is Used to Describe the Synthesis Process of Hydrochloride Salt of Compound 73

The preparation method of this preparation example is similar to that of preparation example 1, with the following differences:

(2S)-1,4-dioxane-2-methanol in step 6) was replaced with an equimolar amount of 2,4-dimethylbenzyl alcohol to obtain hydrochloride salt of compound 73, a white solid, with a one-step yield of 56% in step 6).

¹H NMR (600 MHz, Methanol-d₄) δ 8.89 (s, 1H), 8.69 (s, 1H), 8.05 (d, J=7.8 Hz, 1H), 7.81 (s, 1H), 7.68 (s, 1H), 7.27 (d, J=7.8 Hz, 1H), 7.11-6.96 (m, 3H), 6.84 (d, J=8.4 Hz, 1H), 6.76 (d, J=11.4 Hz, 1H), 6.42 (s, 1H), 5.10 (s, 2H), 4.10 (s, 2H), 2.71 (s, 3H), 2.33 (d, J=26.8 Hz, 6H).

### Preparation Example 74: The Preparation Example is Used to Describe the Synthesis Process of Compound 74

The preparation method of this preparation example is similar to that of preparation example 1, with the following differences:

(2S)-1,4-dioxane-2-methanol in step 6) was replaced with an equimolar amount of 4-hydroxymethylpyridine to obtain compound 74, a white solid, with a one-step yield of 57% in step 6).

¹H NMR (400 MHz, DMSO-d₆) δ 8.87-8.84 (m, 1H), 8.64-8.59 (m, 2H), 8.56 (d, J=2.4 Hz, 1H), 7.88-7.82 (m, 1H), 7.59 (dd, J=8.4, 4.8 Hz, 1H), 7.49-7.43 (m, 3H), 7.04 (t, J=8.4 Hz, 1H), 6.96 (dd, J=11.2, 2.4 Hz, 1H), 6.88 (dd, J=8.4, 2.4 Hz, 1H), 6.31 (d, J=2.0 Hz, 1H), 5.27 (s, 2H), 3.48 (s, 2H), 2.23 (s, 3H).

### Preparation Example 75: The Preparation Example is Used to Describe the Synthesis Process of Hydrochloride Salt of Compound 75

The preparation method of this preparation example is similar to that of preparation example 1, with the following differences:

(2S)-1,4-dioxane-2-methanol in step 6) was replaced with an equimolar amount of 2-methoxybenzyl alcohol to obtain hydrochloride salt of compound 75, a white solid, with a one-step yield of 49% in step 6).

¹H NMR (600 MHz, Methanol-d₄) δ 8.89 (d, J=4.8 Hz, 1H), 8.70 (s, 1H), 8.07 (d, J=5.4 Hz, 1H), 7.83 (s, 1H), 7.70 (dd, J=8.4, 4.8 Hz, 1H), 7.46-7.40 (m, 1H), 7.38-7.32 (m, 1H), 7.09-6.95 (m, 3H), 6.77 (dd, J=8.4, 2.4 Hz, 1H), 6.68 (dd, J=11.4, 2.4 Hz, 1H), 6.43 (s, 1H), 5.17 (s, 2H), 4.11 (s, 2H), 3.90 (s, 3H), 2.72 (s, 3H).

### Preparation Example 76: The Preparation Example is Used to Describe the Synthesis Process of Hydrochloride Salt of Compound 76

The preparation method of this preparation example is similar to that of preparation example 1, with the following differences:

(2S)-1,4-dioxane-2-methanol in step 6) was replaced with an equimolar amount of (R)-1-(2,6-dichloro-3-fluorophenyl)ethanol to obtain hydrochloride salt of compound 76, a white solid, with a one-step yield of 44% in step 6).

¹H NMR (600 MHz, Methanol-d₄) δ 8.85 (d, J=4.8 Hz, 1H), 8.62 (d, J=2.4 Hz, 1H), 7.97 (d, J=8.4 Hz, 1H), 7.77 (s, 1H), 7.68-7.62 (m, 1H), 7.46-7.41 (m, 1H), 7.24 (t, J=8.5 Hz, 1H), 6.94 (t, J=8.4 Hz, 1H), 6.65 (dd, J=8.4, 2.4 Hz, 1H), 6.55 (dd, J=11.4, 2.4 Hz, 1H), 6.36 (d, J=1.8 Hz, 1H), 6.12 (q, J=6.6 Hz, 1H), 4.05 (s, 2H), 2.67 (s, 3H), 1.79 (d, J=6.6 Hz, 3H).

### Preparation Example 77: The Preparation Example is Used to Describe the Synthesis Process of Compound 77

The preparation method of this preparation example is similar to that of preparation example 1, with the following differences:

(2S)-1,4-dioxane-2-methanol in step 6) was replaced with an equimolar amount of 3-hydroxymethylthiophene to obtain compound 77, a yellow solid, with a one-step yield of 40% in step 6).

¹H NMR (600 MHz, DMSO-d₆) δ 8.86 (d, J=4.8 Hz, 1H), 8.57 (s, 1H), 7.85 (d, J=8.4 Hz, 1H), 7.66-7.46 (m, 4H), 7.22 (d, J=4.8 Hz, 1H), 7.00 (t, J=8.4 Hz, 1H), 6.93 (dd, J=11.4, 2.4 Hz, 1H), 6.85 (dd, J=8.4, 2.4 Hz, 1H), 6.32 (s, 1H), 5.16 (s, 2H), 3.55 (s, 2H), 2.27 (s, 3H).

### Preparation Example 78: The Preparation Example is Used to Describe the Synthesis Process of Hydrochloride Salt of Compound 78

The preparation method of this preparation example is similar to that of preparation example 1, with the following differences:

Pyridine-3-sulfonyl chloride in step 3) was replaced with an equimolar amount of 3-chlorobenzenesulfonyl chloride, and (2S)-1,4-dioxane-2-methanol in step 6) was replaced with an equimolar amount of 2,5-difluorobenzyl alcohol to obtain hydrochloride salt of compound 78, a white solid, with a one-step yield of 40% in step 6).

¹H NMR (600 MHz, Methanol-d₄) δ 7.75 (s, 1H), 7.68 (d, J=8.4 Hz, 1H), 7.52-7.45 (m, 1H), 7.43 (d, J=8.4 Hz, 1H), 7.33-7.30 (m, 2H), 7.24-7.17 (m, 1H), 7.17-7.12 (m, 1H), 7.05-7.00 (m, 1H), 6.87 (d, J=9.0 Hz, 1H), 6.78 (d, J=10.8 Hz, 1H), 6.37 (s, 1H), 5.21 (s, 2H), 4.09 (s, 2H), 2.71 (s, 3H).

### Preparation Example 79: The Preparation Example is Used to Describe the Synthesis Process of Hydrochloride Salt of Compound 79

The preparation method of this preparation example is similar to that of preparation example 1, with the following differences:

Pyridine-3-sulfonyl chloride in step 3) was replaced with an equimolar amount of 3-fluorobenzenesulfonyl chloride, and (2S)-1,4-dioxane-2-methanol in step 6) was replaced with an equimolar amount of 2,5-difluorobenzyl alcohol to obtain hydrochloride salt of compound 79, a white solid, with a one-step yield of 36% in step 6).

¹H NMR (600 MHz, Methanol-d₄) δ 7.75 (s, 1H), 7.54-7.48 (m, 1H), 7.47-7.41 (m, 1H), 7.35-7.28 (m, 2H), 7.23-7.11 (m, 3H), 7.02 (t, J=8.4 Hz, 1H), 6.85 (dd, J=8.4, 2.4 Hz, 1H), 6.81-6.76 (m, 1H), 6.38 (s, 1H), 5.20 (s, 2H), 4.09 (s, 2H), 2.71 (s, 3H).

### Preparation Example 80: The Preparation Example is Used to Describe the Synthesis Process of Hydrochloride Salt of Compound 80

The preparation method of this preparation example is similar to that of preparation example 1, with the following differences:

Pyridine-3-sulfonyl chloride in step 3) was replaced with an equimolar amount of 3, 5-difluorobenzenesulfonyl chloride, and (2S)-1,4-dioxane-2-methanol in step 6) was replaced with an equimolar amount of 2,5-difluorobenzyl alcohol to obtain hydrochloride salt of compound 80, a white solid, with a one-step yield of 44% in step 6).

¹H NMR (600 MHz, Methanol-d₄) δ 7.76 (s, 1H), 7.41-7.36 (m, 1H), 7.33-7.29 (m, 1H), 7.22-7.17 (m, 1H), 7.16-7.11 (m, 1H), 7.10-7.04 (m, 3H), 6.88 (d, J=8.4 Hz, 1H), 6.81 (d, J=10.8 Hz, 1H), 6.42 (s, 1H), 5.20 (s, 2H), 4.10 (s, 2H), 2.71 (s, 3H).

### Preparation Example 81: The Preparation Example is Used to Describe the Synthesis Process of Hydrochloride Salt of Compound 81

The preparation method of this preparation example is similar to that of preparation example 1, with the following differences:

Pyridine-3-sulfonyl chloride in step 3) was replaced with an equimolar amount of 3-fluoro-4-methylbenzenesulfonyl chloride, and (2S)-1,4-dioxane-2-methanol in step 6) was replaced with an equimolar amount of 2,5-difluorobenzyl alcohol to obtain hydrochloride salt of compound 81, a white solid, with a one-step yield of 36% in step 6).

¹H NMR (600 MHz, Methanol-d₄) δ 7.73 (s, 1H), 7.37-7.29 (m, 2H), 7.25-7.12 (m, 3H), 7.09 (d, J=9.0 Hz, 1H), 7.06-7.00 (m, 1H), 6.87-6.84 (m, 1H), 6.79 (dd, J=11.4, 2.4 Hz, 1H), 6.36 (s, 1H), 5.20 (s, 2H), 4.08 (s, 2H), 2.70 (s, 3H), 2.31 (s, 3H).

### Preparation Example 82: The Preparation Example is Used to Describe the Synthesis Process of Hydrochloride Salt of Compound 82

The preparation method of this preparation example is similar to that of preparation example 1, with the following differences:

Pyridine-3-sulfonyl chloride in step 3) was replaced with an equimolar amount of 3-pentyloxybenzenesulfonyl chloride, and (2S)-1,4-dioxane-2-methanol in step 6) was replaced with an equimolar amount of 2,5-difluorobenzyl alcohol to obtain hydrochloride salt of compound 82, a white solid, with a one-step yield of 36% in step 6).

¹H NMR (600 MHz, Methanol-d₄) δ 7.74 (s, 1H), 7.40-7.29 (m, 2H), 7.24-7.12 (m, 3H), 7.06 (d, J=7.8 Hz, 1H), 7.03-6.98 (m, 1H), 6.89-6.75 (m, 3H), 6.35 (s, 1H), 5.19 (s, 2H), 4.08 (s, 2H), 3.91-3.81 (m, 2H), 2.70 (s, 3H), 1.82-1.62 (m, 2H), 1.51-1.28 (m, 4H), 0.97-0.87 (m, 3H).

### Preparation Example 83: The Preparation Example is Used to Describe the Synthesis Process of Hydrochloride Salt of Compound 83

The preparation method of this preparation example is similar to that of preparation example 1, with the following differences:

Pyridine-3-sulfonyl chloride in step 3) was replaced with an equimolar amount of 3-cyclobutylmethoxybenzenesulfonyl chloride, and (2S)-1,4-dioxane-2-methanol in step 6) was replaced with an equimolar amount of 1-(2,6-difluorophenyl)-ethanol to obtain hydrochloride salt of compound 83, a yellow oily substance, with a one-step yield of 41% in step 6).

¹H NMR (400 MHz, Methanol-d₄) δ 7.60 (d, J=2.0 Hz, 1H), 7.29-7.19 (m, 1H), 7.13 (t, J=8.4 Hz, 1H), 7.01 (dd, J=8.4, 2.4 Hz, 1H), 6.92-6.75 (m, 4H), 6.72 (t, J=2.4 Hz, 1H), 6.57 (dd, J=8.4, 2.4 Hz, 1H), 6.49 (dd, J=11.4, 2.4 Hz, 1H), 6.21 (d, J=1.8 Hz, 1H), 5.71 (q, J=6.4 Hz, 1H), 3.91 (s, 2H), 3.72 (d, J=6.4 Hz, 2H), 2.54 (s, 3H), 2.07-1.95 (m, 3H), 1.92-1.71 (m, 4H), 1.66 (d, J=6.4 Hz, 3H).

### Preparation Example 84: The Preparation Example is Used to Describe the Synthesis Process of Hydrochloride Salt of Compound 84

The preparation method of this preparation example is similar to that of preparation example 1, with the following differences:

Pyridine-3-sulfonyl chloride in step 3) was replaced with an equimolar amount of 3-cyclopentylmethoxybenzenesulfonyl chloride, and (2S)-1,4-dioxane-2-methanol in step 6) was replaced with an equimolar amount of 2,4-difluorobenzyl alcohol to obtain hydrochloride salt of compound 84, a white solid, with a one-step yield of 40% in step 6).

¹H NMR (400 MHz, Methanol-d₄) δ 7.75 (d, J=1.6 Hz, 1H), 7.40-7.28 (m, 2H), 7.24-7.10 (m, 3H), 7.10-7.06 (m, 1H), 7.01 (t, J=8.4 Hz, 1H), 6.86-6.81 (m, 2H), 6.78 (dd, J=11.2, 2.4 Hz, 1H), 6.36 (d, J=1.6 Hz, 1H), 5.19 (s, 2H), 4.09 (s, 2H), 3.75 (d, J=6.8 Hz, 2H), 2.70 (s, 3H), 2.40-2.22 (m, 1H), 1.90-1.74 (m, 2H), 1.69-1.51 (m, 4H), 1.42-1.27 (m, 2H).

### Preparation Example 85: The Preparation Example is Used to Describe the Synthesis Process of Hydrochloride Salt of Compound 85

The preparation method of this preparation example is similar to that of preparation example 1, with the following differences:

Pyridine-3-sulfonyl chloride in step 3) was replaced with an equimolar amount of 3-cyclopentylmethoxybenzenesulfonyl chloride, and (2S)-1,4-dioxane-2-methanol in step 6) was replaced with an equimolar amount of 1-(2,6-difluorophenyl)-ethanol to obtain hydrochloride salt of compound 85, a yellow oily substance, with a one-step yield of 23% in step 6).

¹H NMR (400 MHz, Methanol-d₄) δ 7.67 (d, J=2.0 Hz, 1H), 7.42-7.31 (m, 1H), 7.25 (t, J=8.0 Hz, 1H), 7.14 (dd, J=8.4, 2.4 Hz, 1H), 7.05-6.95 (m, 2H), 6.94-6.87 (m, 2H), 6.80 (t, J=2.0 Hz, 1H), 6.69 (dd, J=8.4, 2.4 Hz, 1H), 6.59 (dd, J=11.2, 2.4 Hz, 1H), 6.26 (d, J=2.0 Hz, 1H), 5.84 (q, J=6.4 Hz, 1H), 4.01 (s, 2H), 3.75 (d, J=6.8 Hz, 2H), 2.65 (s, 3H), 2.37-2.25 (m, 1H), 1.90-1.75 (m, 5H), 1.72-1.58 (m, 4H), 1.42-1.32 (m, 2H).

### Preparation Example 86: The Preparation Example is Used to Describe the Synthesis Process of Hydrochloride Salt of Compound 86

The preparation method of this preparation example is similar to that of preparation example 1, with the following differences:

Pyridine-3-sulfonyl chloride in step 3) was replaced with an equimolar amount of 3-(2-methoxy-ethoxy)benzenesulfonyl chloride, and (2S)-1,4-dioxane-2-methanol in step 6) was replaced with an equimolar amount of 2,4-difluorobenzyl alcohol to obtain hydrochloride salt of compound 86, a white solid, with a one-step yield of 45% in step 6).

¹H NMR (400 MHz, Methanol-d₄) δ 7.75 (d, J=2.0 Hz, 1H), 7.41-7.29 (m, 2H), 7.25-7.11 (m, 3H), 7.12-7.05 (m, 1H), 7.00 (t, J=8.4 Hz, 1H), 6.88 (t, J=2.0 Hz, 1H), 6.84 (dd, J=8.4, 2.4 Hz, 1H), 6.78 (dd, J=11.2, 2.4 Hz, 1H), 6.36 (d, J=2.0 Hz, 1H), 5.20 (s, 2H), 4.09 (s, 2H), 4.06-4.01 (m, 2H), 3.74-3.67 (m, 2H), 3.38 (s, 3H), 2.70 (s, 3H).

### Preparation Example 87: The Preparation Example is Used to Describe the Synthesis Process of Compound 87

The preparation method of this preparation example is similar to that of preparation example 1, with the following differences:

Pyridine-3-sulfonyl chloride in step 3) was replaced with an equimolar amount of 3-(2-methoxy-ethoxy)benzenesulfonyl chloride, and (2S)-1,4-dioxane-2-methanol in step 6) was replaced with an equimolar amount of 2-methoxybenzyl alcohol to obtain compound 87, a white solid, with a one-step yield of 39% in step 6).

¹H NMR (400 MHz, Methanol-d₄) δ 7.68 (d, J=2.0 Hz, 1H), 7.46-7.40 (m, 1H), 7.38-7.30 (m, 2H), 7.20 (dd, J=8.4, 2.4 Hz, 1H), 7.07-7.02 (m, 2H), 7.00-6.92 (m, 2H), 6.87 (t, J=2.0 Hz, 1H), 6.78 (dd, J=8.4, 2.4 Hz, 1H), 6.71 (dd, J=11.2, 2.4 Hz, 1H), 6.31 (d, J=2.0 Hz, 1H), 5.15 (s, 2H), 4.04-3.98 (m, 4H), 3.89 (s, 3H), 3.71-3.65 (m, 2H), 3.36 (s, 3H), 2.65 (s, 3H).

### Preparation Example 88: The Preparation Example is Used to Describe the Synthesis Process of Compound 88

The preparation method of this preparation example is similar to that of preparation example 1, with the following differences:

Pyridine-3-sulfonyl chloride in step 3) was replaced with an equimolar amount of 3-(3-methoxy-propoxy)benzenesulfonyl chloride, and (2S)-1,4-dioxane-2-methanol in step 6) was replaced with an equimolar amount of 1-(2,6-difluorophenyl)-ethanol to obtain compound 88, a yellow oily substance, with a one-step yield of 44% in step 6).

¹H NMR (400 MHz, Methanol-d₄) δ 7.67 (d, J=2.0 Hz, 1H), 7.42-7.31 (m, 1H), 7.25 (t, J=8.0 Hz, 1H), 7.18-7.12 (m, 1H), 7.04-6.96 (m, 2H), 6.94-6.87 (m, 2H), 6.83 (t, J=2.0 Hz, 1H), 6.70 (dd, J=8.8, 2.4 Hz, 1H), 6.60 (dd, J=11.2, 2.4 Hz, 1H), 6.27 (d, J=2.0 Hz, 1H), 5.85 (q, J=6.4 Hz, 1H), 4.01 (s, 2H), 3.96 (t, J=6.4 Hz, 2H), 3.54 (t, J=6.0 Hz, 2H), 3.35 (s, 3H), 2.64 (s, 3H), 2.04-1.95 (m, 2H), 1.79 (d, J=6.4 Hz, 3H).

### Preparation Example 89: The Preparation Example is Used to Describe the Synthesis Process of Hydrochloride Salt of Compound 89

The preparation method of this preparation example is similar to that of preparation example 1, with the following differences:

Pyridine-3-sulfonyl chloride in step 3) was replaced with an equimolar amount of 3-(3-methoxy-propoxy)benzenesulfonyl chloride, and (2S)-1,4-dioxane-2-methanol in step 6) was replaced with an equimolar amount of 1-(3,5-difluorophenyl)-ethanol to obtain hydrochloride salt of compound 89, a white solid, with a one-step yield of 46% in step 6).

¹H NMR (400 MHz, Methanol-d₄) δ 7.63 (d, J=2.0 Hz, 1H), 7.21 (t, J=8.0 Hz, 1H), 7.08 (dd, J=8.4, 2.4 Hz, 1H), 7.01-6.94 (m, 2H), 6.93-6.88 (m, 1H), 6.86-6.74 (m, 3H), 6.62 (dd, J=8.4, 2.4 Hz, 1H), 6.55 (dd, J=11.2, 2.4 Hz, 1H), 6.23 (d, J=2.0 Hz, 1H), 5.44 (q, J=6.4 Hz, 1H), 3.98 (s, 2H), 3.89 (t, J=6.4 Hz, 2H), 3.46 (t, J=6.0 Hz, 2H), 3.27 (s, 3H), 2.60 (s, 3H), 1.96-1.85 (m, 2H), 1.55 (d, J=6.4 Hz, 3H).

### Preparation Example 90: The Preparation Example is Used to Describe the Synthesis Process of Hydrochloride Salt of Compound 90

The preparation method of this preparation example is similar to that of preparation example 1, with the following differences:

Pyridine-3-sulfonyl chloride in step 3) was replaced with an equimolar amount of 3-(4-methoxy-butoxy)benzenesulfonyl chloride, and (2S)-1,4-dioxane-2-methanol in step 6) was replaced with an equimolar amount of 2-hydroxymethylnaphthalene to obtain hydrochloride salt of compound 90, a white solid, with a one-step yield of 51% in step 6).

¹H NMR (600 MHz, Methanol-d₄) δ 8.08 (d, J=8.4 Hz, 1H), 7.94-7.85 (m, 2H), 7.73-7.69 (m, 1H), 7.64 (d, J=6.6 Hz, 1H), 7.59-7.45 (m, 3H), 7.28 (t, J=7.8 Hz, 1H), 7.13 (dd, J=8.4, 2.4 Hz, 1H), 7.03 (d, J=7.8 Hz, 1H), 6.97 (t, J=8.4 Hz, 1H), 6.85 (dd, J=8.4, 2.4 Hz, 1H), 6.81 (d, J=11.4 Hz, 2H), 6.32 (d, J=2.4 Hz, 1H), 5.56 (s, 2H), 4.05 (s, 2H), 3.85 (t, J=6.0 Hz, 2H), 3.26 (s, 3H), 3.21-3.18 (m, 2H), 2.67 (s, 3H), 1.75-1.68 (m, 2H), 1.65-1.58 (m, 2H).

### Preparation Example 91: The Preparation Example is Used to Describe the Synthesis Process of Compound 91

The preparation method of this preparation example is similar to that of preparation example 1, with the following differences:

Pyridine-3-sulfonyl chloride in step 3) was replaced with an equimolar amount of 3-(4-methoxy-butoxy)benzenesulfonyl chloride, and (2S)-1,4-dioxane-2-methanol in step 6) was replaced with an equimolar amount of 1-(2,6-difluorophenyl)-ethanol to obtain compound 91, a white solid, with a one-step yield of 48% in step 6).

¹H NMR (400 MHz, Methanol-d₄) δ 7.69 (d, J=2.0 Hz, 1H), 7.42-7.32 (m, 1H), 7.25 (t, J=8.0 Hz, 1H), 7.15 (dd, J=8.4, 2.4 Hz, 1H), 7.04-6.96 (m, 2H), 6.95-6.86 (m, 2H), 6.82 (t, J=2.0 Hz, 1H), 6.70 (dd, J=8.4, 2.4 Hz, 1H), 6.60 (dd, J=11.4, 2.4 Hz, 1H), 6.29 (d, J=2.0 Hz, 1H), 5.84 (q, J=6.4 Hz, 1H), 4.04 (s, 2H), 3.90 (t, J=6.4 Hz, 2H), 3.46 (t, J=6.4 Hz, 2H), 3.34 (s, 3H), 2.66 (s, 3H), 1.88-1.67 (m, 7H).

### Preparation Example 92: The Preparation Example is Used to Describe the Synthesis Process of Hydrochloride Salt of Compound 92

The preparation method of this preparation example is similar to that of preparation example 1, with the following differences:

Pyridine-3-sulfonyl chloride in step 3) was replaced with an equimolar amount of 3-(4-methoxy-butoxy)benzenesulfonyl chloride, and (2S)-1,4-dioxane-2-methanol in step 6) was replaced with an equimolar amount of 1-(3,5-difluorophenyl)-ethanol to obtain hydrochloride salt of compound 92, a yellow solid, with a one-step yield of 41% in step 6).

¹H NMR (400 MHz, Methanol-d₄) δ 7.71 (d, J=2.0 Hz, 1H), 7.29 (t, J=8.0 Hz, 1H), 7.16 (dd, J=8.4, 2.54 Hz, 1H), 7.09-7.02 (m, 2H), 7.01-6.96 (m, 1H), 6.95-6.84 (m, 2H), 6.82 (t, J=2.0 Hz, 1H), 6.70 (dd, J=8.4, 2.4 Hz, 1H), 6.63 (dd, J=11.2, 2.4 Hz, 1H), 6.31 (d, J=2.0 Hz, 1H), 5.52 (q, J=6.4 Hz, 1H), 4.06 (s, 2H), 3.91 (t, J=6.4 Hz, 2H), 3.45 (t, J=6.4 Hz, 2H), 3.34 (s, 3H), 2.68 (s, 3H), 1.85-1.76 (m, 2H), 1.76-1.68 (m, 2H), 1.63 (d, J=6.3 Hz, 3H).

### Preparation Example 93: The Preparation Example is Used to Describe the Synthesis Process of Hydrochloride Salt of Compound 93

The preparation method of this preparation example is similar to that of preparation example 1, with the following differences:

Pyridine-3-sulfonyl chloride in step 3) was replaced with an equimolar amount of 3-(4-methoxy-butoxy)benzenesulfonyl chloride, and (2S)-1,4-dioxane-2-methanol in step 6) was replaced with an equimolar amount of 1,3-benzodioxolane-4-alkylmethanol to obtain hydrochloride salt of compound 93, a white solid, with a one-step yield of 40% in step 6).

¹H NMR (400 MHz, Methanol-d₄) δ 7.73 (d, J=2.0 Hz, 1H), 7.33 (t, J=8.0 Hz, 1H), 7.17 (dd, J=8.4, 2.4 Hz, 1H), 7.04 (d, J=8.0 Hz, 1H), 7.00-6.94 (m, 2H), 6.92-6.77 (m, 4H), 6.73 (dd, J=11.2, 2.4 Hz, 1H), 6.34 (d, J=2.0 Hz, 1H), 6.01 (s, 2H), 5.11 (s, 2H), 4.08 (s, 2H), 3.90 (t, J=6.4 Hz, 2H), 3.41 (t, J=6.4 Hz, 2H), 3.30 (s, 3H), 2.70 (s, 3H), 1.86-1.75 (m, 2H), 1.74-1.63 (m, 2H).

### Preparation Example 94: The Preparation Example is Used to Describe the Synthesis Process of Hydrochloride Salt of Compound 94

The preparation method of this preparation example is similar to that of preparation example 1, with the following differences:

(2S)-1,4-dioxane-2-methanol in step 6) was replaced with an equimolar amount of 1,3-benzodioxolane-4-alkylmethanol to obtain hydrochloride salt of compound 94, a white solid, with a one-step yield of 50% in step 6).

¹H NMR (600 MHz, DMSO-d₆) δ 9.14 (s, 2H), 8.89 (d, J=3.6 Hz, 1H), 8.59 (s, 1H), 7.89 (d, J=8.4 Hz, 1H), 7.81 (s, 1H), 7.63-7.59 (m, 1H), 7.01-6.94 (m, 4H), 6.91-6.85 (m, 2H), 6.52 (s, 1H), 6.08 (s, 2H), 5.11 (s, 2H), 3.98 (s, 3H).

### Preparation Example 95: The Preparation Example is Used to Describe the Synthesis Process of Hydrochloride Salt of Compound 95

The preparation method of this preparation example is similar to that of preparation example 1, with the following differences:

Pyridine-3-sulfonyl chloride in step 3) was replaced with an equimolar amount of 3-cyano-benzenesulfonyl chloride, and (2S)-1,4-dioxane-2-methanol in step 6) was replaced with an equimolar amount of 1,3-benzodioxolane-4-alkylmethanol to obtain hydrochloride salt of compound 95, a white solid, with a one-step yield of 44% in step 6).

¹H NMR (400 MHz, Methanol-d₄) δ 7.73 (d, J=2.0 Hz, 1H), 7.33 (t, J=8.0 Hz, 1H), 7.17 (dd, J=8.4, 2.4 Hz, 1H), 7.04 (d, J=8.0 Hz, 1H), 7.00-6.94 (m, 2H), 6.92-6.77 (m, 4H), 6.73 (dd, J=11.2, 2.4 Hz, 1H), 6.34 (d, J=2.0 Hz, 1H), 6.01 (s, 2H), 5.11 (s, 2H), 4.08 (s, 2H), 3.90 (t, J=6.4 Hz, 2H), 3.41 (t, J=6.4 Hz, 2H), 3.30 (s, 3H), 2.70 (s, 3H), 1.86-1.75 (m, 2H), 1.74-1.63 (m, 2H).

### Preparation Example 96: The Preparation Example is Used to Describe the Synthesis Process of Hydrochloride Salt of Compound 96

The preparation method of this preparation example is similar to that of preparation example 1, with the following differences:

(2S)-1,4-dioxane-2-methanol in step 6) was replaced with an equimolar amount of 2,3-dihydro-1,4-benzodioxin-5-methanol to obtain hydrochloride salt of compound 96, a white solid, with a one-step yield of 56% in step 6).

¹H NMR (600 MHz, Methanol-d₄) δ 8.96-8.86 (m, 1H), 8.71 (d, J=2.4 Hz, 1H), 8.15-8.01 (m, 1H), 7.82 (d, J=2.4 Hz, 1H), 7.71 (dd, J=8.4, 4.8 Hz, 1H), 7.06-6.97 (m, 2H), 6.87-6.80 (m, 3H), 6.75 (dd, J=11.4, 2.4 Hz, 1H), 6.43 (d, J=2.4 Hz, 1H), 5.13 (s, 2H), 4.37-4.24 (m, 4H), 4.11 (s, 2H), 2.72 (s, 3H).

### Preparation Example 97: The Preparation Example is Used to Describe the Synthesis Process of Hydrochloride Salt of Compound 97

The preparation method of this preparation example is similar to that of preparation example 1, with the following differences:

Pyridine-3-sulfonyl chloride in step 3) was replaced with an equimolar amount of 3-cyano-benzenesulfonyl chloride, and (2S)-1,4-dioxane-2-methanol in step 6) was replaced with an equimolar amount of 2,3-dihydro-1,4-benzodioxin-5-methanol to obtain hydrochloride salt of compound 97, a white solid, with a one-step yield of 43% in step 6).

¹H NMR (600 MHz, Methanol-d₄) δ 8.03 (d, J=7.7 Hz, 1H), 7.81-7.60 (m, 4H), 7.00 (d, J=10.4 Hz, 2H), 6.88-6.80 (m, 3H), 6.73 (d, J=11.4 Hz, 1H), 6.40 (s, 1H), 5.15 (s, 2H), 4.37-4.25 (m, 4H), 4.10 (s, 2H), 2.72 (s, 3H).

### Preparation Example 98: The Preparation Example is Used to Describe the Synthesis Process of Hydrochloride Salt of Compound 98

The preparation method of this preparation example is similar to that of preparation example 1, with the following differences:

Pyridine-3-sulfonyl chloride in step 3) was replaced with an equimolar amount of 3-trifluoromethylbenzenesulfonyl chloride, and (2S)-1,4-dioxane-2-methanol in step 6) was replaced with an equimolar amount of 2,5-difluorobenzyl alcohol to obtain hydrochloride salt of compound 98, a white solid, with a one-step yield of 52% in step 6).

¹H NMR (600 MHz, Methanol-d₄) δ 8.00 (d, J=7.8 Hz, 1H), 7.80 (d, J=5.4 Hz, 2H), 7.76-7.70 (m, 1H), 7.60 (s, 1H), 7.34-7.27 (m, 1H), 7.23-7.17 (m, 1H), 7.17-7.12 (m, 1H), 7.03 (t, J=8.4 Hz, 1H), 6.85 (d, J=8.4 Hz, 1H), 6.75 (d, J=11.4 Hz, 1H), 6.39 (s, 1H), 5.19 (s, 2H), 4.09 (s, 2H), 2.70 (s, 3H).

### Preparation Example 99: The Preparation Example is Used to Describe the Synthesis Process of Hydrochloride Salt of Compound 99

The preparation method of this preparation example is similar to that of preparation example 1, with the following differences:

(2S)-1,4-dioxane-2-methanol in step 6) was replaced with an equimolar amount of 2-(3,5-difluorophenyl)-ethanol to obtain hydrochloride salt of compound 99, a white solid, with a one-step yield of 48% in step 6). ¹H NMR (600 MHz, Methanol-d₄) δ 8.80 (d, J=4.8 Hz, 1H), 8.55 (d, J=2.4 Hz, 1H), 7.98-7.92 (m, 1H), 7.76 (s, 1H), 7.59 (dd, J=8.4, 4.8 Hz, 1H), 7.00-6.89 (m, 3H), 6.82-6.75 (m, 1H), 6.73 (d, J=8.4 Hz, 1H), 6.64 (d, J=11.4 Hz, 1H), 6.36 (t, J=1.8 Hz, 1H), 4.24 (t, J=6.0 Hz, 2H), 4.06 (s, 2H), 3.10 (t, J=6.0 Hz, 2H), 2.67 (s, 3H).

### Preparation Example 100: The Preparation Example is Used to Describe the Synthesis Process of Hydrochloride Salt of Compound 100

The preparation method of this preparation example is similar to that of preparation example 1, with the following differences:

(2S)-1,4-dioxane-2-methanol in step 6) was replaced with an equimolar amount of 3-hydroxymethylfuran to obtain hydrochloride salt of compound 100, a grey solid, with a one-step yield of 51% in step 6).

¹H NMR (600 MHz, Methanol-d₄) δ 8.89 (d, J=4.8 Hz, 1H), 8.67 (s, 1H), 8.07 (d, J=8.4 Hz, 1H), 7.82 (s, 1H), 7.71 (dd, J=8.4, 4.8 Hz, 1H), 7.65 (s, 1H), 7.53 (s, 1H), 7.02 (t, J=8.4 Hz, 1H), 6.86-6.81 (m, 1H), 6.75 (d, J=11.4 Hz, 1H), 6.55 (s, 1H), 6.45-6.39 (m, 1H), 5.04 (s, 2H), 4.10 (s, 2H), 2.71 (s, 3H).

### Test Example 1: Biological Evaluation of H⁺/K⁺-ATPase

This test example evaluates the inhibitory effects of the compounds of the present invention on the activity of H+/K+-ATPase by an in vitro screening test, and expresses them with the IC₅₀ values of the compounds (obtained by calculating the inhibition rates at different concentrations).

### In Vitro Screening Test:

### (1) Reagent Preparation:

Compound solutions: The products (i.e., the compounds or the hydrochloride salt of compounds) obtained in the foregoing preparation examples of the present invention were each independently dissolved in DMSO to form solutions at suitable concentrations;
Buffer I: 50 mmol/L HEPEs-Tris, pH=6.5, 5 mmol/L magnesium chloride, 10 mol/L valinomycin (J&K Scientific Ltd., goods No.: 227304);
Buffer II: 50 mmol/L HEPEs-Tris, pH=6.5, 5 mmol/L magnesium chloride, 10 mol/L valinomycin, 5 mmol/L potassium chloride;
5-adenosine triphosphate (ATP, Sigma-Aldrich, goods No.: A2383) solution: Dilute ATP with buffer I to 5 mM;
Malachite green solution: Dissolve 0.12 wt % malachite green (J&K Scientific Ltd., goods No.: 913120) in 2.5 mol/L sulfuric acid, 7.5 wt % ammonium molybdate (J&K Scientific Ltd., goods No.: 128321) and 11% Tween 20(V/V). During use, sulfuric acid, ammonium molybdate and Tween 20 are mixed in a ratio of 100:25:2;
Rabbit gastric mucosal microsomes (rich in H+/K+-ATPase, self-extracted), extracted by sucrose gradient centrifugation: The rabbit stomach was washed with tap water and 3M NaCl solution, respectively, and then the surface water was removed with filter paper. A pre-cooled homogenization buffer (4 ml/g tissue) was added and homogenized for 2-5 min in a tissue homogenizer. After the homogenizing, if there are large tissue particles, they can be removed by centrifugation (600 g, 10 min). The supernatant was then transferred to a clean centrifuge tube and centrifuged at 20,000 g for 30 min, then the supernatant was transferred to a clean centrifuge tube and further centrifuged at 100,000 g for 90 min and the precipitate was collected; the precipitate was suspended in homogenate and blown evenly, and the protein concentration was measured by the Bradford's method and adjusted to 10 mg/ml; 7.5 wt % Ficoll's layering solution was added in equal proportions. After centrifugation at 100,000 g for 60 min, the middle layer (H⁺/K⁺-ATPase-enriched gastric membranes) was collected in a clean centrifuge tube, diluted with homogenate by 4-5 times, and then centrifuged at 100,000 g for another 90 min, and the precipitate was collected; the precipitate was suspended in homogenate and homogenized in a glass homogenizer, and the protein concentration was measured by the Bradford's method and adjusted to 22.5 mg/ml. It was frozen at −80° C. and stored for future use.

### (2) Experimental Process:

5 μL of rabbit gastric mucosal microsomes (H⁺/K⁺-ATPase) was added to 45 μL of buffer II, then 5 μL of compound solution was added, and then 5 μL of 5 mM ATP solution was added to start the reaction and pre-react at 37° C. for 30 min. 15 μL of malachite green solution was added to terminate the reaction, balancing was conducted at room temperature for 20 min, and read the optical absorbance at 620 nm.

Meanwhile, the reaction of the same volume was carried out without addition of potassium chloride as a background, which was subtracted when calculating enzymatic activity. The results are shown in Table 1.

TABLE 1
Inhibitory Effects of the Compounds on H⁺/K⁺-ATPase Activity

| Compound | IC₅₀ | Compound | IC₅₀ | Compound | IC₅₀ | Compound | IC₅₀ |
|---|---|---|---|---|---|---|---|
| No. | (nM) | No. | (nM) | No. | (nM) | No. | (nM) |
| A1 | 66.7 | A5 | 169.2 | A9 | 33.8 | A12 | 118.7 |
| A2 | 154.8 | A6 | 156.9 | A10 | 27.7 | A13 | 127.9 |
| A3 | 51.6 | A8 | 18.42 | A11 | 14.1 | — | — |
| 1 | 585.3 | 26 | 201.7 | 51 | 35.2 | 76 | 470.1 |
| 2 | 24.5 | 27 | 38.7 | 52 | 372.3 | 77 | 119.6 |
| 3 | 20.9 | 28 | 30.8 | 53 | 56.1 | 78 | 98.5 |
| 4 | 30.9 | 29 | 289.9 | 54 | 17.8 | 79 | 55.3 |
| 5 | 82.4 | 30 | 474.5 | 55 | 8.6 | 80 | 65.9 |
| 6 | 149.2 | 31 | 1211 | 56 | 13.3 | 81 | 56.6 |
| 7 | 253.5 | 32 | 821.8 | 57 | 46.3 | 82 | 3659 |
| 8 | 72.1 | 33 | 883.4 | 58 | 25.7 | 83 | 13340 |
| 9 | 25.4 | 34 | 285.2 | 59 | 41.3 | 84 | 109 |
| 10 | 55.3 | 35 | 8649 | 60 | 186.4 | 85 | 9781 |
| 11 | 65.2 | 36 | 2272 | 61 | 51.3 | 86 | 34.4 |
| 12 | 138 | 37 | 157.3 | 62 | 35.9 | 87 | 43.4 |
| 13 | 114.2 | 38 | 198.8 | 63 | 1057 | 88 | 449.8 |
| 14 | 442.7 | 39 | 34.6 | 64 | 163.7 | 89 | 1333 |
| 15 | 83.9 | 40 | 123.2 | 65 | 80.5 | 90 | 2692 |
| 16 | 253 | 41 | 566.6 | 66 | 484.6 | 91 | 692.6 |
| 17 | 264.9 | 42 | 400.9 | 67 | 41 | 92 | 1906 |
| 18 | 261.8 | 43 | 481.6 | 68 | 41.4 | 93 | 12372 |
| 19 | 233.1 | 44 | 500.4 | 69 | 114.3 | 94 | 48.4 |
| 20 | 293.8 | 45 | 203.5 | 70 | 88.1 | 95 | 9.5 |
| 21 | 2787 | 46 | 639.4 | 71 | 496.3 | 96 | 51.1 |
| 22 | 458.4 | 47 | 246.8 | 72 | 342.1 | 97 | 11.9 |
| 23 | 907.8 | 48 | 36 | 73 | 209 | 98 | 484.6 |
| 24 | 135.8 | 49 | 146.1 | 74 | 564.4 | 99 | 72.6 |
| 25 | 58.5 | 50 | 113.5 | 75 | 36.6 | 100 | 101.2 |

As can be seen from Table 1, the compounds of the present invention have excellent inhibitory effects on the activity of H⁺/K⁺-ATPase.

### Test Example 2: Inhibitory Effects of the Compounds on Histamine-Induced Gastric Acid Secretion in Rats

70 SPF-level SD rats with a weight of 180-220 g (Hangzhou Medical College (produced)), randomly grouped by weight, including negative control groups (isovolumetric saline) and model control groups (isovolumetric saline), and compounds obtained in the foregoing preparation examples of the present invention (i.e., compounds or compound hydrochlorides) (2 mg/kg or 4 mg/kg), 10 rats each group, including 5 males and 5 females. The rats were fasted, but were allowed to drink water for 24 h. After 24 h, the rats were given the compound at 1 ml/100 g by gavage, once in each group, and the rats in the negative control groups and the model groups were given an equal volume of saline by gavage. After that, the rats were anesthetized with chloral hydrate 300 mg/kg (1 ml/100 g) and fixed on a rat plate, and the abdominal wall was incised along the abdominal midline from the subxiphoid process of the sternum, and the incision was approximately 2-3 cm and was made on the left costal margin, and the stomach was exposed to the incision by gently pushing it with the fingers upwards. The pylorus was ligated with a thread under the pylorus (other neighboring vessels were not ligated), and the abdominal wall incision was sutured. The animals were given histamine dihydrochloride (30 mg/10 ml/kg) subcutaneously after 1 h of gavage of the test compound or saline. After 3 h of histamine administration, the rats were asphyxiated with excess CO₂, stomachs were removed, stomach contents were collected and centrifuged at 3000 rpm/min for 10 min, and the acid solution was titrated with 0.1 mol/L NaOH to pH7.0. The total acidity and acid inhibition rate during the 3 h period were calculated.

The acid inhibition rate was calculated by the following formula, and the results are shown in Table 2:

Acid inhibition rate (%)=(Total acid in the model group−total acid in the test group)/total acid in the model group×100%

TABLE 2
Inhibitory Effects of the Compounds on Histamine-
Induced Gastric Acid Secretion in Rats

| Compound No. | Dose (mg/kg) | Acid inhibition rate (%) |
|---|---|---|
| 6 | 2 | 48 |
| 8 | 2 | 71 |
| 28 | 2 | 71 |
| 37 | 2 | 56 |
| 48 | 2 | 75 |
| 48 | 4 | 86 |
| 67 | 2 | 64 |
| A1 | 2 | 68 |
| A3 | 2 | 65 |
| A4 | 2 | 60 |
| A7 | 2 | 53 |
| A8 | 2 | 58 |
| A9 | 2 | 83 |
| A9 | 4 | 93 |
| A10 | 2 | 81 |
| A10 | 4 | 87 |
| A11 | 2 | 81 |
| A12 | 2 | 63 |
| A13 | 2 | 60 |
| A14 | 2 | 63 |
| A15 | 2 | 64 |

Table 2 shows that the compounds of the present invention have good inhibitory effects on histamine-induced gastric acid secretion in rats.

### Test Example 3: Rat Tissue Distribution Test for Compound 48, Compound A9 and Comparison Compound Vonoprazan

Three healthy male SD rats (body weight range 220-250 g, Zhejiang Charles River) were taken and assigned into one group (n=3). Compound 48, Compound A9 or comparison compound Vonoprazan were administered orally at a dose of 4 mg/kg.

Solvent for preparing pharmaceutical preparations: Pure water. The rats were fasted 4 h prior to the administration. Plasma, liver, and stomach were collected at 1 h, 2 h, and 4 h (for the remaining SD rats, blank tissues were collected for the analysis group). The tissue was homogenized, and the corresponding blank tissue was used for preparation of a calibration curve and QC quantification. Blood samples should be centrifuged to extract plasma within 1 hour of collection and kept on crushed ice prior to the centrifugation. Plasma samples obtained by centrifugation were stored in a freezer until analysis. Centrifugation conditions: 4-10° C., 8000 rpm, 6 min. The concentration of the sample was determined by LC-MS-MS (MS03: Shimadzu LC30AD and API 4000)), and the exposure data were calculated by WINNOLIN software. The results are shown in Tables 3, 4 and 5.

TABLE 3
Tissue Distribution of Rats Orally Administered
Compound 48 at a Dose of 4 mg/kg

| | Exposure level | |
|---|---|---|
| Tissue | (0-4 h)/h*ng/mL | Ratio |
| Plasma | 85.8 | 1 |
| Liver | 7585.5 | 88.4 |
| Stomach | 14693.7 | 171.3 |

TABLE 4
Tissue Distribution of Rats Orally Administered
CompoundA9 at a Dose of 4 mg/kg

| | Exposure level | |
|---|---|---|
| Tissue | (0-4 h)/h*ng/mL | Ratio |
| Plasma | 43.1 | 0.02 |
| Liver | 2326.8 | 1 |
| Stomach | 13224.3 | 5.7 |

Tables 3 and 4 show that the compounds of the present invention have a very obvious enrichment effect in gastric tissues, while the exposure levels in plasma are low, and the exposure levels in liver are significantly lower than those in the stomach.

TABLE 5
Comparison between Compound A9 and Comparison
Compound Vonoprazan at an Oral Dose of 4 mg/kg
in Terms of Hepatic Drug Concentration

time point

| Compound | 15 min | 1 h | 2 h | 4 h | 24 h |
|---|---|---|---|---|---|
| A9 | 1541.3 | 846 | 262 | 31.5 | 1.3 |
| Vonoprazan | 9919 | 1192 | 632 | 162 | 23.7 |

Table 5 shows that the compounds of the present invention had significantly lower compound concentrations in the liver at various time points than the comparison compound Vonoprazan.

The data in Table 3, Table 4 and Table 5 show that the compounds of the present invention are expected to improve the therapeutic effect and at the same time reduce the hepatotoxicity and other safety risks of the marketed drug Vonoprazan.

Preferred embodiments of the present invention have been described in detail above, but the present invention is not limited thereto. Within the scope of the technical concept of the present invention, various simple changes can be made to the technical schemes of the present invention, including the combinations of various technical features in any other suitable manner, and these simple changes and combinations shall also be regarded as the contents disclosed in the present invention and shall fall within the scope of protection of the present invention.

1.-10. (canceled)

11. A compound containing a sulfamide structure, or its tautomer, mesomer, racemate, enantiomer or diastereoisomer or a mixture thereof, or a pharmaceutically acceptable salt thereof, wherein the compound has a structure shown in formula (I), 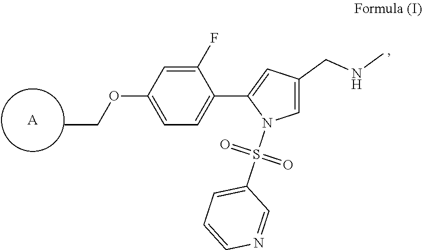
in formula (I),
ring A is selected from substituted or unsubstituted saturated 3-membered carbon ring, saturated 3-membered ring containing O atom, saturated 4-membered ring containing O atom, saturated 5-membered ring containing O atom, substituted unsaturated 5-membered ring containing at least one nitrogen atom, and substituted unsaturated 6-membered ring containing at least one nitrogen atom;
the substituents in ring A are each independently selected from halogen, cyano, nitro, C₁-C₆ alkyl and C₁-C₆ alkoxy.

12. The compound according to claim 11, wherein in formula (I),
ring A is selected from substituted or unsubstituted saturated 3-membered carbon ring, saturated 3-membered ring containing O atom, saturated 4-membered ring containing O atom, saturated 5-membered ring containing O atom, substituted unsaturated 5-membered ring containing at least one nitrogen atom, and substituted unsaturated 6-membered ring containing at least one nitrogen atom;
the substituents in ring A are each independently selected from halogen, C₁-C₄ alkyl and C₁-C₄ alkoxy.

13. The compound according to claim 12, wherein in formula (I),
ring A is selected from substituted or unsubstituted saturated 3-membered carbon ring, saturated 3-membered ring containing O atom, saturated 4-membered ring containing O atom, saturated 5-membered ring containing O atom, substituted unsaturated 5-membered ring containing at least one nitrogen atom, and substituted unsaturated 6-membered ring containing at least one nitrogen atom;
the substituents in ring A are each independently selected from halogen, methyl, ethyl, n-propyl, isopropyl, n-butyl, isobutyl, tert-butyl, methoxy, ethoxy, n-propoxy, isopropoxy, n-butoxy, isobutoxy and tert-butoxy.

14. The compound according to claim 13, wherein in formula (I),
ring A is selected from substituted or unsubstituted saturated 3-membered carbon ring, saturated 3-membered ring containing O atom, saturated 4-membered ring containing O atom, saturated 5-membered ring containing O atom, substituted unsaturated 5-membered ring containing at least one nitrogen atom, and substituted unsaturated 6-membered ring containing at least one nitrogen atom;
the substituents in ring A are each independently selected from fluorine, chlorine, bromine, methyl, ethyl, n-propyl, isopropyl, n-butyl, isobutyl, tert-butyl, methoxy, ethoxy, n-propoxy, isopropoxy, n-butoxy, isobutoxy and tert-butoxy.

15. The compound according to any of claim 14, wherein the compound with a structure shown in formula (I) is selected from any one of the following:

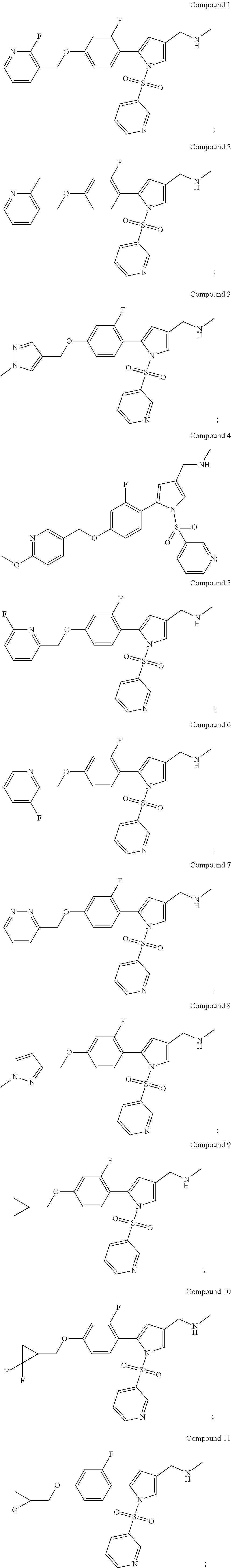

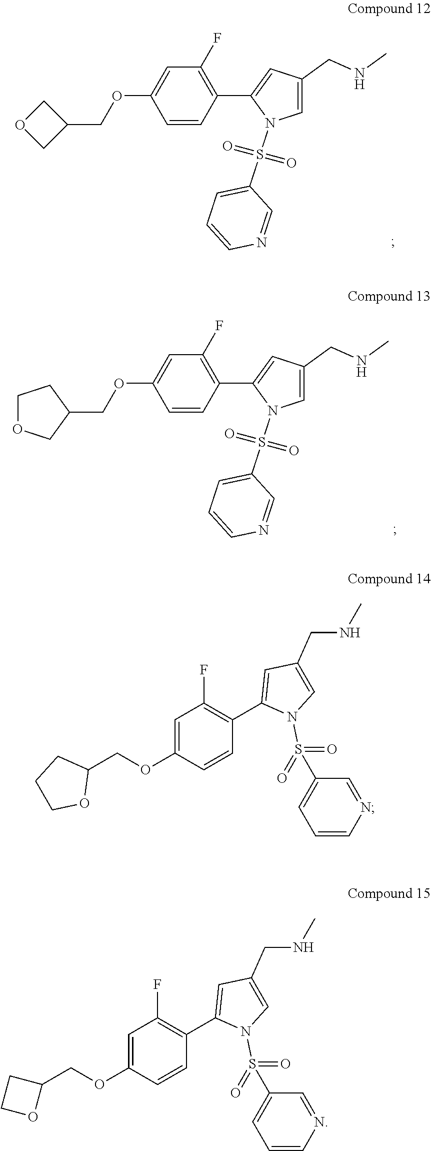 [SMILES: CNCc1cc(-c2ccc(OCC3COC3)cc2F)n(S(=O)(=O)c2cccnc2)c1 ; CNCc1cc(-c2ccc(OCC3CCOC3)cc2F)n(S(=O)(=O)c2cccnc2)c1 ; CNCc1cc(-c2ccc(OCC3CCCO3)cc2F)n(S(=O)(=O)c2cccnc2)c1 ; CNCc1cc(-c2ccc(OCC3CCO3)cc2F)n(S(=O)(=O)c2cccnc2)c1]

16. A method for preparing a compound containing a sulfamide structure, or its tautomer, mesomer, racemate, enantiomer or diastereoisomer or a mixture thereof, or a pharmaceutically acceptable salt thereof, wherein the compound has a structure shown in formula (I), 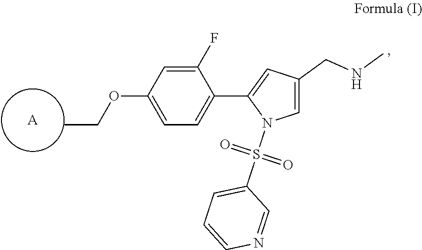
the method comprises: performing a contact reaction between a compound shown in formula (II) and a compound shown in formula (III); 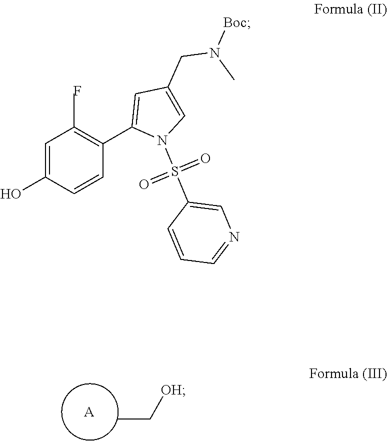
wherein in formula (I) and formula (III), the definition of ring A is the same as that described in claim 11.

17. A pharmaceutical composition, wherein the pharmaceutical composition contains a therapeutically effective amount of the compound, or its tautomer, mesomer, racemate, enantiomer or diastereoisomer or a mixture thereof, or a pharmaceutically acceptable salt thereof in claim 11, and the pharmaceutical composition further contains a pharmaceutically acceptable carrier, excipient or diluent.
